(19) United States
(12) Patent Application Publication
(10) Pub. No.: US 2010/0261708 A1
(43) Pub. Date: Oct. 14, 2010

(54) Diazepane Compounds Which Modulate The CB2 Receptor

(75) Inventors: Pier Francesco Cirillo, Woodbury, CT (US); Eugene Richard Hickey, Danbury, CT (US); Doris Riether, Newtown, CT (US); Lifen Wu, New Milford, CT (US); Renee M. Zindel, New Milford, CT (US); Patricia Amouzegh, Didcot (GB); Monika Ermann, Wantage (GB); Edward Richard Walker, Didcot (GB)

Correspondence Address: MICHAEL P. MORRIS;BOEHRINGER INGELHEIM USA CORPORATION, 900 RIDGEBURY ROAD, P O BOX 368, RIDGEFIELD, CT, 06877-0368, US

(73) Assignee: BOEHRINGER INGELHEIM INTERNATIONAL GMBH, Ingelheim am Rhein (DE)

(21) Appl. No.: 12/738,899

(22) PCT Filed: Oct. 21, 2008
(86) PCT No.: PCT/US08/80591

§ 371 Date: May 12, 2010

Related U.S. Application Data
(60) Provisional application No. 60/982,502, filed on Oct. 25, 2007.

(51) Int. Cl. A61K 31/551; C07D 243/08; A61P 29/00; A61P 11/00; A61P 37/06; A61P 37/08; A61P 9/00; A61P 17/00; A61P 13/12; A61P 1/16; A61P 1/00; A61P 27/02; A61P 25/00; A61P 7/00; A61P 5/00; A61P 35/00; A61P 9/10

(52) U.S. Cl. 514/218; 540/575

(57) ABSTRACT

Compounds of formula (I) are disclosed. Compounds according to the invention bind to and are agonists, antagonists or inverse agonists of the CB2 receptor, and are useful for treating inflammation. Those compounds which are agonists are additionally useful for treating pain.

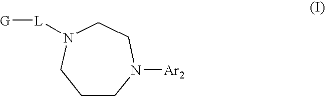

### APPLICATION DATA

This application claims benefit to U.S. provisional application Ser. No. 60/982,502 filed Oct. 25, 2007.

### BACKGROUND OF THE INVENTION

1. Technical Field

The present invention relates to novel compounds which modulate the CB2 receptor and their use as medicaments.

2. Background Information

Cannabinoids are a group of about 60 distinct compounds found in Cannabis sativa (also know as marijuana) with cannabinol, cannabidiol and Δ⁹-tetrahydrocannabinol (THC) being the most representative molecules. The therapeutic usage of Cannabis can be dated back to ancient dynasties of China and includes applications for various illnesses ranging from lack of appetite, emesis, cramps, menstrual pain, spasticity to rheumatism. The long history of Cannabis use has led to the development of several pharmaceutical drugs. For example, Marinol and Cesamet which are based on THC and its analogous nabilone, respectively, are used as anti-emetic and appetite stimulant. Despite of the clinical benefits, the therapeutic usage of cannabis is limited by its psychoactive effects including hallucination, addiction and dependence. Mechoulam R, ed. Cannabinoids as Therapeutic Agents, Boca Raton, Fla.; CRC Press, 1986 provides a review of the medicinal use of cannabis.

The physiological effects of cannabinoids are mediated by at least two G-protein coupled receptors, CB1 and CB2. Autoradiographic studies have demonstrated that CB1 receptors are expressed primarily in the central nervous system, specifically in the cerebral cortex, hippocampus, basal ganglia and cerebellum. They are also found to a lesser degree in the reproductive system and other peripheral tissues including that of the immune system. CB1 receptors regulate the release of neurotransmitters from the pre-synaptic neurons and are believed to mediate most of the euphoric and other central nervous system effects of cannabis, such as THC-induced ring-catalepsy, hypomobility, and hypothermia, which were found to be completely absent in mice with a deletion of the CB1 gene (Zimmer et al., Increased mortality, hypoactivity, and hypoalgesia in cannabinoid CB1 receptor knockout mice. Proc Natl Acad Sci USA. (1999) 96:5780-5785.)

CB2 receptors are almost exclusively found in the immune system, with the greatest density in the spleen. It is estimated that the expression level of CB2 in the immune cells is about 10 to 100 times higher than CB1. Within the immune system, CB2 is found in various cell types, including B cells, NK cells, monocytes, microglial cells, neutrophils, T cells, dentritic cells and mast cells, suggesting that a wide range of immune functions can be regulated through CB2 modulators (Klein et al., The cannabinoid system and immune system. J Leukoc Biol (2003) 74:486-496). This is supported by the finding that the immunomodulatory effect of THC is absent in CB2 deficient mice mice (Bicklet et al., Immunomodulation by cannabinoid is absent in mice deficient for the cannabinoid CB2 receptor. Eur J Pharmacol (2000) 396:141-149). CB2 selective ligands have been developed and tested for their effects in various settings. For example, in animal models of inflammation, CB2 selective agonists, inverse agonists and antagonists have been shown to be effective in suppressing inflammation (Hanus et al., HU-308: a specific agonist for CB(2), a peripheral cannabinoid receptor. Proc Natl Acad Sci USA. (1999) 96:14228-14233, Ueda et al., Involvement of cannabinoid CB(2) receptor-mediated response and efficacy of cannabinoid CB(2) receptor inverse agonist, JTE-907, in cutaneous inflammation in mice. Eur J Pharmacol. (2005) 520:164-171 and Smith et al., The anti-inflammatory activities of cannabinoid receptor ligands in mouse peritonitis models Eur J Pharmacol. (2001) 432:107-119.). Furthermore, CB2 selective agonists inhibit disease severity and spasticity in animal models for multiple sclerosis (Baker et al., Cannabinoids control spasticity and tremor in a multiple sclerosis model. Nature (2000) 404:84-87. Arevalo-Martin et al., Therapeutic action of cannabinoids in a murine model of multiple sclerosis J Neurosci. (2003) 23:2511-2516.). Taken together, these results support the notion that CB2 receptor modulators can be employed for the treatment of medical conditions having an inflammatory component.

In addition to inflammation, CB2 agonists have been shown to inhibit pain and emesis. For instance, CB2 selective agonists blunt the pain response induced by thermal or other stimuli (Malan et al., CB2 cannabinoid receptor-mediated peripheral antinociception. Pain. (2001) 93:239-45 and Nackley et al., Selective activation of cannabinoid CB(2) receptors suppresses spinal fos protein expression and pain behavior in a rat model of inflammation. Neuroscience (2003) 119:747-57.) CB2 activation has also been demonstrated to inhibit neuropathic pain response (Ibrahim et al., Activation of CB2 cannabinoid receptors by AM1241 inhibits experimental neuropathic pain: pain inhibition by receptors not present in the CNS. Proc Natl Acad Sci USA. (2003) 100:10529-33.) Finally, in contrast to the earlier data which did not find CB2 in the brain, a recent article demonstrated the expression of CB2 in the brain, at about 1.5% of the level in the spleen. CB2 activation is shown by this article to be responsible for the anti-emetic effect of endocannabinoid (Van Sickle et al., Identification and functional characterization of brainstem cannabinoid CB2 receptors. Science. 2005 310:329-332.) The foregoing results confirm that CB2 agonists can be used for the treatment of inflammatory and neuropathic pain as well as emesis.

### BRIEF SUMMARY OF THE INVENTION

The present invention provides novel compounds which bind to and modulate the CB2 receptor. The invention also provides a method and pharmaceutical compositions for treating inflammation by way of the administration of therapeutic amounts of these compounds. Lastly, the invention provides a method and pharmaceutical compositions for treating pain by way of the administration of therapeutic amounts of the new compounds which are CB2 agonists.

### DETAILED DESCRIPTION OF THE INVENTION

In its broadest generic aspect the invention provides compounds of the formula

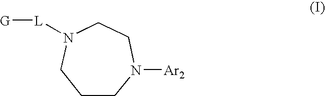

wherein:

L is a bond, —C(O), —CH₂—C(O)— or —NH—C(O)—;

G is chosen from a C1-10 alkyl branched or unbranched wherein one or more methylene groups may be replaced by NH, O or S(O)ₘ, Ar₁(CH₂)₀₋₂— and Ar₁;

Ar₁ is chosen from carbocycle, benzoxazolyl, benzothiazolyl, benzimidazolyl, tetrahydropyranyl, dioxanyl, tetrahydrofuranyl, oxazolyl, isoxazolyl, thiazolyl, pyrazolyl, pyrrolyl, imidazolyl, thienyl, thiadiazolyl, thiomorpholinyl, 1,1-dioxo-1λ⁶-thiomorpholinyl, morpholinyl, pyridinyl, pyrimidinyl, pyridazinyl, pyrazinyl, triazinyl, pyrrolidinyl, piperidinyl and piperazinyl each optionally substituted by one to three R₁;

Ar₂ is chosen from carbocycle, benzoxazolyl, benzothiazolyl, benzimidazolyl, purinyl, quinolinyl, isoquinolinyl, quinazolinyl, indazolyl, thieno[2,3-d]pyrimidinyl, indolyl, isoindolyl, benzofuranyl, benzopyranyl, benzodioxolyl, tetrahydropyranyl, dioxanyl, tetrahydrofuranyl, oxazolyl, isoxazolyl, thiazolyl, pyrazolyl, pyrrolyl, imidazolyl, thienyl, thiadiazolyl, thiomorpholinyl, thiomorpholinyl, morpholinyl, pyridinyl, pyrimidinyl, pyridazinyl, pyrazinyl, triazinyl, pyrrolidinyl, piperidinyl and piperazinyl each optionally substituted by one to three R₂;

m is 0, 1 or 2;

R₁ is C₁₋₁₀ alkyl, halogen, oxo (═O), C₁₋₁₀ alkoxy, carbocycle, C₁-C₆ acyl, C₁-C₆ acylamino, hydroxyl, amino, nitro, cyano, C₁-C₄ mono- or di-alkylaminoC₀-C₄alkyl and phenyl, each R₁ where possible is optionally partially or fully halogenated;

R₂ is C₁-C₁₀ alkyl, halogen, carbocycle, cyano or C₁₋₁₀ alkoxy, each R₂ is optionally partially or fully halogenated and optionally substituted by carbocycle-C₀₋₂ alkyl or a heteroring-C₀₋₂ alkyl wherein the heteroring is chosen from

benzoxazolyl, benzothiazolyl, benzimidazolyl, purinyl, quinolinyl, dihydro-2H-quinolinyl, isoquinolinyl, quinazolinyl, indazolyl, thieno[2,3-d]pyrimidinyl, indolyl, isoindolyl, benzofuranyl, benzopyranyl, benzodioxolyl, tetrahydropyranyl, dioxanyl, tetrahydrofuranyl, oxazolyl, isoxazolyl, thiazolyl, pyrazolyl, pyrrolyl, imidazolyl, thienyl, thiadiazolyl, thiomorpholinyl, thiomorpholinyl, morpholinyl, pyridinyl, pyrimidinyl, pyridazinyl, pyrazinyl, triazinyl, pyrrolidinyl, piperidinyl and piperazinyl each R₂ carbocycle or heteroring optionally independently substituted with 1 to 3 substituents chosen from C₁-C₆ alkyl, C₁-C₆ alkoxy, C₁-C₆ acyl, C₁-C₆ acylamino, C₃-C₆ cycloalkyl, phenoxy, halogen, hydroxyl, amino, nitro, cyano, C₁-C₄ mono- or di-alkylaminoC₀-C₄alkyl and phenyl;

or a pharmaceutically acceptable salt thereof.

In a first subgeneric aspect, the invention provides compounds of the formula I wherein,

Ar₁ is chosen from benzoxazolyl, benzothiazolyl, benzimidazolyl, cyclohexyl, cyclopentyl, phenyl, tetrahydropyranyl, dioxanyl, tetrahydrofuranyl, oxazolyl, isoxazolyl, thiazolyl, pyrazolyl, pyrrolyl, imidazolyl, thienyl, thiadiazolyl, thiomorpholinyl, 1,1-Dioxo-1λ⁶-thiomorpholinyl, morpholinyl, pyridinyl, pyrimidinyl, pyridazinyl, pyrazinyl, triazinyl, pyrrolidinyl, piperidinyl and piperazinyl each optionally substituted by one to three R₁;

Ar₂ is chosen from phenyl, benzoxazolyl, benzothiazolyl, benzimidazolyl, purinyl, quinolinyl, isoquinolinyl, thieno[2,3-d]pyrimidinyl, oxazolyl, isoxazolyl, thiazolyl, pyrazolyl, thiadiazolyl, pyridinyl, pyrimidinyl, pyridazinyl, pyrazinyl, triazinyl, piperidinyl and piperazinyl each optionally substituted by one to three R₂;

R₁ is C₁₋₁₀ alkyl, halogen or oxo;

R₂ is C₁-C₁₀ alkyl, halogen, carbocycle, cyano or C₁₋₁₀ alkoxy, each R₂ is optionally partially or fully halogenated and optionally substituted by phenyl, benzyl or a heteroring-C₀₋₂ alkyl wherein the heteroring is chosen from

quinolinyl, dihydro-2H-quinolinyl, isoquinolinyl, oxazolyl, isoxazolyl, thiazolyl, pyrazolyl, pyrrolyl, imidazolyl, thiadiazolyl, thiomorpholinyl, thiomorpholinyl, morpholinyl, pyridinyl, pyrimidinyl, pyridazinyl, pyrazinyl, triazinyl, pyrrolidinyl, piperidinyl and piperazinyl each R₂ ring is optionally independently substituted with 1 to 3 substituents chosen from C₁-C₆ alkyl, C₁-C₆ alkoxy, C₁-C₆ acyl, C₁-C₆ acylamino, C₃-C₆ cycloalkyl, phenoxy, halogen, hydroxyl, amino, nitro, cyano, C₁-C₄ mono- or di-alkylaminoC₀-C₄alkyl and phenyl.

In a further subgeneric aspect, the invention provides compounds of the formula I wherein,

R₁ is C₁₋₆ alkyl, halogen or oxo;

R₂ is C₁-C₁₀ alkyl, halogen, phenyl, cyano or C₁₋₁₀ alkoxy, each R₂ is optionally partially or fully halogenated and optionally substituted by quinolinyl, dihydro-2H-quinolinyl, morpholinyl, each R₂ ring optionally independently substituted with 1 to 3 substituents chosen from C₁-C₆ alkyl, C₁-C₆ alkoxy, halogen and cyano.

In another subgeneric aspect, the invention provides compounds of the formula I wherein,

G is chosen from

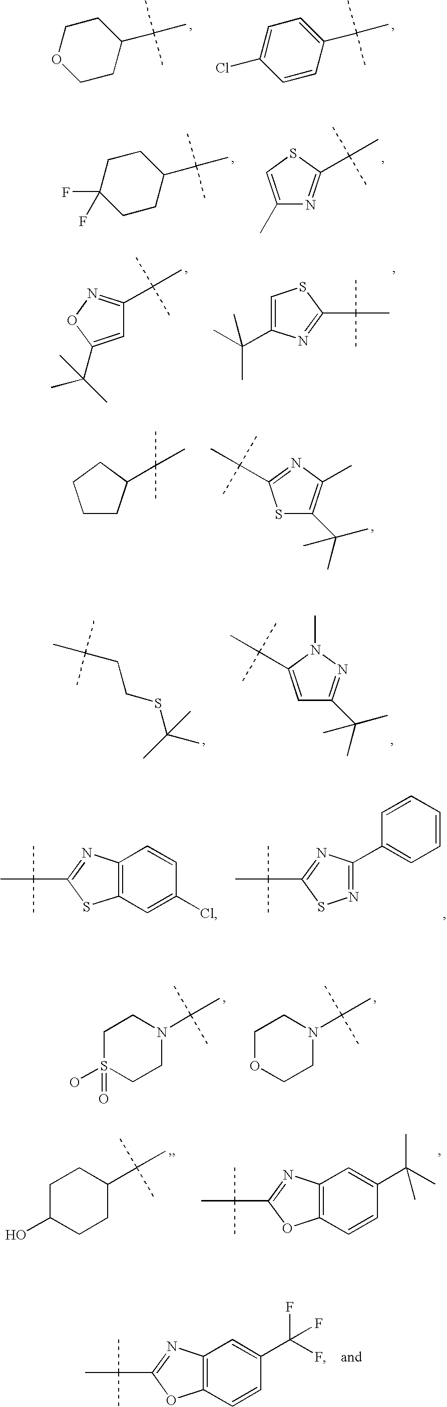

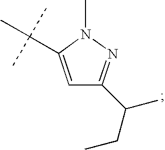

Ar₂ is chosen from:

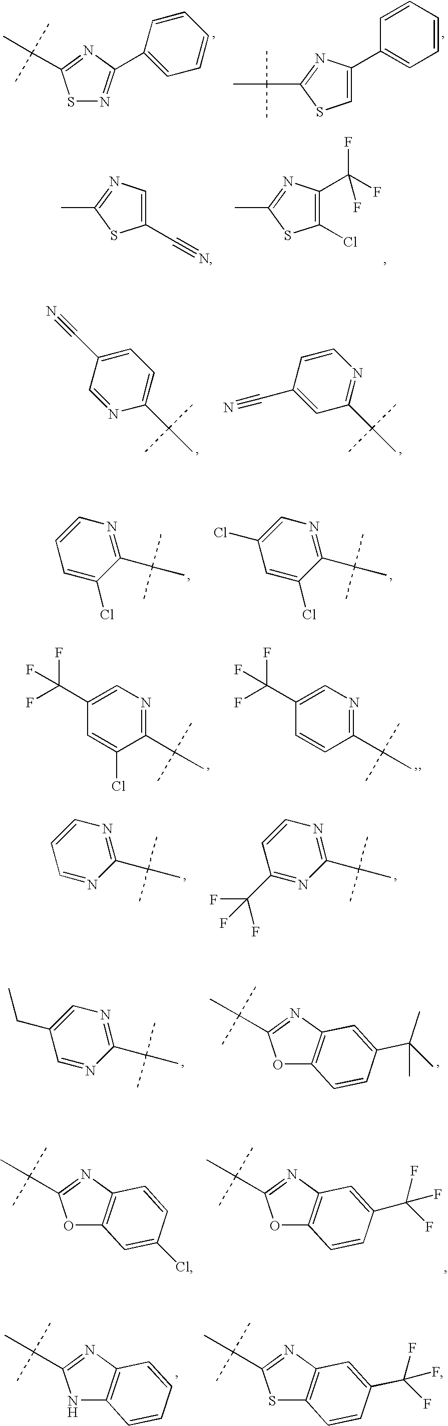

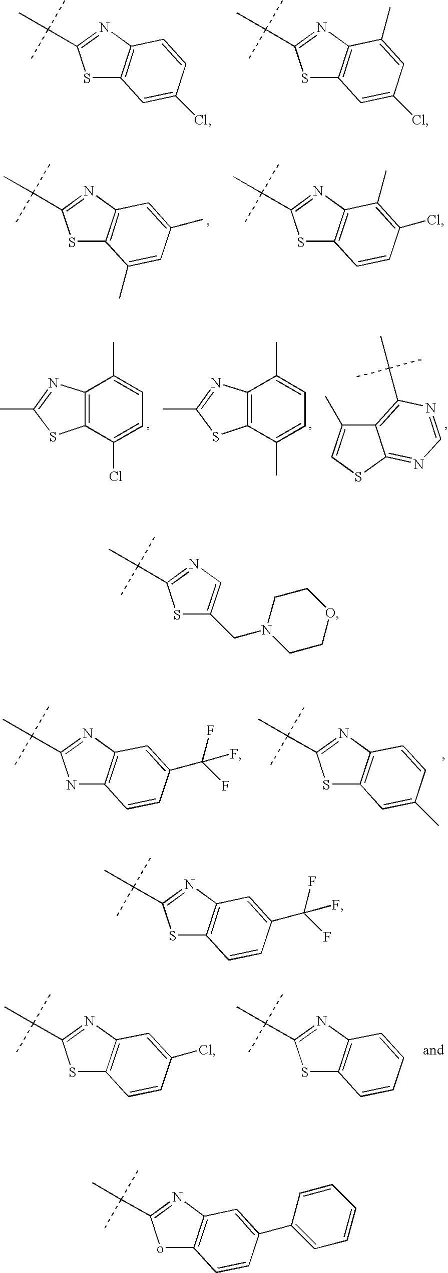

In another embodiment, there is provided compounds of the formula (I) as described below which can be made as described in the schemes and examples herein below, and by methods apparent to those of ordinary skill in the art:
1-[4-(5-tert-Butyl-benzooxazol-2-yl)-[1,4]diazepan-1-yl]-2-cyclopentyl-ethanone;
[4-(5-tert-Butyl-benzooxazol-2-yl)-[1,4]diazepan-1-yl]-(tetrahydro-pyran-4-yl)-methanone;
[4-(5-tert-Butyl-benzooxazol-2-yl)-[1,4]diazepan-1-yl]-(4-chloro-phenyl)-methanone;
[4-(5-tert-Butyl-benzooxazol-2-yl)-[1,4]diazepan-1-yl]-(4,4-difluoro-cyclohexyl)-methanone;
(Tetrahydro-pyran-4-yl)-[4-(5-trifluoromethyl-benzooxazol-2-yl)-[1,4]diazepan-1-yl]-methanone;
4-(3-Chloro-5-trifluoromethyl-pyridin-2-yl)-[1,4]diazepane-1-carboxylic acid (4-methyl-thiazol-2-yl)-amide;
4-(3-Chloro-5-trifluoromethyl-pyridin-2-yl)-[1,4]diazepane-1-carboxylic acid (tetrahydro-pyran-4-ylmethyl)-amide;
[4-(6-Chloro-benzothiazol-2-yl)-[1,4]diazepan-1-yl]-(tetrahydro-pyran-4-yl)-methanone;
[4-(6-Chloro-benzothiazol-2-yl)-[1,4]diazepan-1-yl]-(4-chloro-phenyl)-methanone;
[4-(3-Phenyl-[1,2,4]thiadiazol-5-yl)-[1,4]diazepan-1-yl]-(tetrahydro-pyran-4-yl)-methanone;
(4-Chloro-phenyl)-[4-(3-phenyl-[1,2,4]thiadiazol-5-yl)-[1,4]diazepan-1-yl]-methanone;
4-(3-Chloro-5-trifluoromethyl-pyridin-2-yl)-[1,4]diazepane-1-carboxylic acid (5-tert-butyl-isoxazol-3-yl)-amide;
4-(3-Chloro-5-trifluoromethyl-pyridin-2-yl)-[1,4]diazepane-1-carboxylic acid (4-tert-butyl-thiazol-2-yl)-amide;
4-(3-Chloro-5-trifluoromethyl-pyridin-2-yl)-[1,4]diazepane-1-carboxylic acid (5-tert-butyl-4-methyl-thiazol-2-yl)-amide;
4-(3-Chloro-5-trifluoromethyl-pyridin-2-yl)-[1,4]diazepane-1-carboxylic acid (2-tert-butylsulfanyl-ethyl)-amide;
4-(3-Chloro-5-trifluoromethyl-pyridin-2-yl)-[1,4]diazepane-1-carboxylic acid (5-tert-butyl-2-methyl-2H-pyrazol-3-yl)-amide;
(4-Trifluoromethyl-phenyl)-[4-(5-trifluoromethyl-pyridin-2-yl)-[1,4]diazepan-1-yl]-methanone;
4-(5-Trifluoromethyl-pyridin-2-yl)-[1,4]diazepane-1-carboxylic acid (4-trifluoromethyl-phenyl)-amide;
6-Chloro-2-[4-(3-chloro-5-trifluoromethyl-pyridin-2-yl)-[1,4]diazepan-1-yl]-benzothiazole;
1-(3-Chloro-5-trifluoromethyl-pyridin-2-yl)-4-(3-phenyl-[1,2,4]thiadiazol-5-yl)-[1,4]diazepane;
4-(3,5-Dichloro-pyridin-2-yl)-[1,4]diazepane-1-carboxylic acid (5-tert-butyl-isoxazol-3-yl)-amide;
4-Pyrimidin-2-yl-[1,4]diazepane-1-carboxylic acid (5-tert-butyl-isoxazol-3-yl)-amide;
4-(4-Trifluoromethyl-pyrimidin-2-yl)-[1,4]diazepane-1-carboxylic acid (5-tert-butyl-isoxazol-3-yl)-amide;
4-(5-Trifluoromethyl-pyridin-2-yl)-[1,4]diazepane-1-carboxylic acid (5-tert-butyl-isoxazol-3-yl)-amide;
4-(3-Chloro-pyridin-2-yl)-[1,4]diazepane-1-carboxylic acid (5-tert-butyl-isoxazol-3-yl)-amide;
4-Pyrimidin-2-yl-[1,4]diazepane-1-carboxylic acid (5-tert-butyl-2-methyl-2H-pyrazol-3-yl)-amide;
4-(5-Trifluoromethyl-pyridin-2-yl)-[1,4]diazepane-1-carboxylic acid (5-tert-butyl-2-methyl-2H-pyrazol-3-yl)-amide;
4-(3,5-Dichloro-pyridin-2-yl)-[1,4]diazepane-1-carboxylic acid (5-tert-butyl-2-methyl-2H-pyrazol-3-yl)-amide;
4-(6-Chloro-9H-purin-2-yl)-[1,4]diazepane-1-carboxylic acid [5-tert-butyl-isoxazol-(3E)-ylidene]-amide;
(1,1-Dioxo-1λ⁶-thiomorpholin-4-yl)-[4-(5-trifluoromethyl-benzooxazol-2-yl)-[1,4]diazepan-1-yl]-methanone;
Morpholin-4-yl-[4-(5-trifluoromethyl-benzooxazol-2-yl)-[1,4]diazepan-1-yl]-methanone;
(4-Hydroxy-cyclohexyl)-[4-(5-trifluoromethyl-benzooxazol-2-yl)-[1,4]diazepan-1-yl]-methanone;
(4-Benzothiazol-2-yl-[1,4]diazepan-1-yl)-morpholin-4-yl-methanone;
2-[4-(Tetrahydro-pyran-4-carbonyl)-[1,4]diazepan-1-yl]-thiazole-5-carbonitrile;
[4-(5-Chloro-4-trifluoromethyl-thiazol-2-yl)-[1,4]diazepan-1-yl]-morpholin-4-yl-methanone;
[4-(5-Chloro-4-trifluoromethyl-thiazol-2-yl)-[1,4]diazepan-1-yl]-(tetrahydro-pyran-4-yl)-methanone;
(4-Benzothiazol-2-yl-[1,4]diazepan-1-yl)-(tetrahydro-pyran-4-yl)-methanone;
[4-(5-Chloro-4-methyl-benzothiazol-2-yl)-[1,4]diazepan-1-yl]-morpholin-4-yl-methanone;
[4-(7-Chloro-4-methyl-benzothiazol-2-yl)-[1,4]diazepan-1-yl]-morpholin-4-yl-methanone;
[4-(5,7-Dimethyl-benzothiazol-2-yl)-[1,4]diazepan-1-yl]-morpholin-4-yl-methanone;
[4-(5-Methoxy-benzothiazol-2-yl)-[1,4]diazepan-1-yl]-morpholin-4-yl-methanone;
[4-(6-Chloro-4-methyl-benzothiazol-2-yl)-[1,4]diazepan-1-yl]-(tetrahydro-pyran-4-yl)-methanone;
[4-(7-Chloro-4-methyl-benzothiazol-2-yl)-[1,4]diazepan-1-yl]-(tetrahydro-pyran-4-yl)-methanone;
[4-(5-Methoxy-benzothiazol-2-yl)-[1,4]diazepan-1-yl]-(tetrahydro-pyran-4-yl)-methanone;
[4-(4-Methyl-benzothiazol-2-yl)-[1,4]diazepan-1-yl]-(tetrahydro-pyran-4-yl)-methanone;
[4-(4,7-Dimethyl-benzothiazol-2-yl)-[1,4]diazepan-1-yl]-(tetrahydro-pyran-4-yl)-methanone;
[4-(5,7-Dimethyl-benzothiazol-2-yl)-[1,4]diazepan-1-yl]-(tetrahydro-pyran-4-yl)-methanone;
[4-(5-Chloro-4-methyl-benzothiazol-2-yl)-[1,4]diazepan-1-yl]-(tetrahydro-pyran-4-yl)-methanone;
2-[4-(3-Chloro-5-trifluoromethyl-pyridin-2-yl)-[1,4]diazepan-1-yl]-5-trifluoromethyl-benzooxazole;
[4-(5-tert-Butyl-benzooxazol-2-yl)-[1,4]diazepan-1-yl]-morpholin-4-yl-methanone;
[4-(5-tert-Butyl-benzooxazol-2-yl)-[1,4]diazepan-1-yl]-(1,1-dioxo-1,6-thiomorpholin-4-yl)-methanone;
[4-(4-Methyl-thiazol-2-yl)-[1,4]diazepan-1-yl]-(tetrahydro-pyran-4-yl)-methanone;
[4-(4-Phenyl-thiazol-2-yl)-[1,4]diazepan-1-yl]-(tetrahydro-pyran-4-yl)-methanone;
[4-(5-Morpholin-4-ylmethyl-thiazol-2-yl)-[1,4]diazepan-1-yl]-(tetrahydro-pyran-4-yl)-methanone;
5-tert-Butyl-2-[4-(3-chloro-5-trifluoromethyl-pyridin-2-yl)-[1,4]diazepan-1-yl]-benzooxazole;
[4-(1H-Benzoimidazol-2-yl)-[1,4]diazepan-1-yl]-(1,1-dioxo-1,6-thiomorpholin-4-yl)-methanone;
[4-(1H-Benzoimidazol-2-yl)-[1,4]diazepan-1-yl]-(tetrahydro-pyran-4-yl)-methanone;
Morpholin-4-yl-[4-(6-trifluoromethyl-1H-benzoimidazol-2-yl)-[1,4]diazepan-1-yl]-methanone;
(1,1-Dioxo-1λ⁶-thiomorpholin-4-yl)-[4-(6-trifluoromethyl-1H-benzoimidazol-2-yl)-[1,4]diazepan-1-yl]-methanone;
(Tetrahydro-pyran-4-yl)-[4-(6-trifluoromethyl-1H-benzoimidazol-2-yl)-[1,4]diazepan-1-yl]-methanone;
[4-(3-Methyl-pyridin-2-yl)-[1,4]diazepan-1-yl]-(tetrahydro-pyran-4-yl)-methanone;
4-(5-Ethyl-pyrimidin-2-yl)-[1,4]diazepane-1-carboxylic acid (5-tert-butyl-2-methyl-2H-pyrazol-3-yl)-amide;
[4-(6-Fluoro-benzothiazol-2-yl)-[1,4]diazepan-1-yl]-(tetrahydro-pyran-4-yl)-methanone;
[4-(6-Methyl-benzothiazol-2-yl)-[1,4]diazepan-1-yl]-(tetrahydro-pyran-4-yl)-methanone;
(Tetrahydro-pyran-4-yl)-[4-(5-trifluoromethyl-benzothiazol-2-yl)-[1,4]diazepan-1-yl]-methanone;
[4-(5-Chloro-benzothiazol-2-yl)-[1,4]diazepan-1-yl]-(tetrahydro-pyran-4-yl)-methanone;
(4,4-Difluoro-cyclohexyl)-[4-(5-trifluoromethyl-benzooxazol-2-yl)-[1,4]diazepan-1-yl]-methanone;
4-(3-Chloro-5-trifluoromethyl-pyridin-2-yl)-[1,4]diazepane-1-carboxylic acid (5-sec-butyl-2-methyl-2H-pyrazol-3-yl)-amide;
[4-(5-Chloro-benzooxazol-2-yl)-[1,4]diazepan-1-yl]-(tetrahydro-pyran-4-yl)-methanone;
[4-(6-Chloro-benzooxazol-2-yl)-[1,4]diazepan-1-yl]-(tetrahydro-pyran-4-yl)-methanone;
[4-(5-Phenyl-benzooxazol-2-yl)-[1,4]diazepan-1-yl]-(tetrahydro-pyran-4-yl)-methanone;
[4-(5-Chloro-benzooxazol-2-yl)-[1,4]diazepan-1-yl]-morpholin-4-yl-methanone;
[4-(6-Chloro-benzooxazol-2-yl)-[1,4]diazepan-1-yl]-morpholin-4-yl-methanone;
Morpholin-4-yl-[4-(5-phenyl-benzooxazol-2-yl)-[1,4]diazepan-1-yl]-methanone;
4-(5-Cyano-pyridin-2-yl)-[1,4]diazepane-1-carboxylic acid (5-tert-butyl-2-methyl-2H-pyrazol-3-yl)-amide;
4-(5-Cyano-pyridin-2-yl)-[1,4]diazepane-1-carboxylic acid (5-tert-butyl-isoxazol-3-yl)-amide;
4-(4,6-Dimethoxy-[1,3,5]triazin-2-yl)-[1,4]diazepane-1-carboxylic acid (5-tert!-butyl-2-methyl-2H-pyrazol-3-yl)-amide;
4-(6-Cyano-pyridin-2-yl)-[1,4]diazepane-1-carboxylic acid (5-tert!-butyl-isoxazol-3-yl)-amide;
4-(5-Ethyl-pyrimidin-2-yl)-[1,4]diazepane-1-carboxylic acid (5-tert-butyl-isoxazol-3-yl)-amide;
4-(4-Cyano-pyridin-2-yl)-[1,4]diazepane-1-carboxylic acid (5-tert-butyl-isoxazol-3-yl)-amide;
4-(4-Methyl-pyrimidin-2-yl)-[1,4]diazepane-1-carboxylic acid (5-tert-butyl-isoxazol-3-yl)-amide;
(4-Furo[3,2-c]pyridin-4-yl-[1,4]diazepan-1-yl)-morpholin-4-yl-methanone;
[4-(4-Fluoro-phenyl)-[1,4]diazepan-1-yl]-morpholin-4-yl-methanone;
[4-(6-Chloro-benzothiazol-2-yl)-[1,4]diazepan-1-yl]-morpholin-4-yl-methanone;
Morpholin-4-yl-[4-(8-trifluoromethyl-quinolin-4-yl)-[1,4]diazepan-1-yl]-methanone;
[4-(5-Methyl-thieno[2,3-d]pyrimidin-4-yl)-[1,4]diazepan-1-yl]-morpholin-4-yl-methanone;
{4-[5-(3,5-Dimethyl-pyrazol-1-ylmethyl)-thiazol-2-yl]-[1,4]diazepan-1-yl}-(tetrahydro-pyran-4-yl)-methanone;
{4-[5-(3,4-Dihydro-2H-quinolin-1-ylmethyl)-thiazol-2-yl]-[1,4]diazepan-1-yl}-(tetrahydro-pyran-4-yl)-methanone;
[4-(5-Methyl-benzooxazol-2-yl)-[1,4]diazepan-1-yl]-(tetrahydro-pyran-4-yl)-methanone; and
[4-(5-Methyl-oxazolo[4,5-b]pyridin-2-yl)-[1,4]diazepan-1-yl]-(tetrahydro-pyran-4-yl)-methanone

or a pharmaceutically acceptable salt thereof.

Of the above compounds, the following are preferred CB2 agonists:

| Compound | CB2 CAMP @EC50 (nM) |
|---|---|
| 1-[4-(5-tert-Butyl-benzooxazol-2-yl)-[1,4]diazepan-1-yl]-2- | 2 |
| cyclopentyl-ethanone | |
| [4-(5-tert-Butyl-benzooxazol-2-yl)-[1,4]diazepan-1-yl]- | 1 |
| (tetrahydro-pyran-4-yl)-methanone | |
| [4-(5-tert-Butyl-benzooxazol-2-yl)-[1,4]diazepan-1-yl]-(4- | 0.14 |
| chloro-phenyl)-methanone | |
| [4-(5-tert-Butyl-benzooxazol-2-yl)-[1,4]diazepan-1-yl]-(4,4- | 0.017 |
| difluoro-cyclohexyl)-methanone | |
| (Tetrahydro-pyran-4-yl)-[4-(5-trifluoromethyl-benzooxazol- | 43 |
| 2-yl)-[1,4]diazepan-1-yl]-methanone | |
| 4-(3-Chloro-5-trifluoromethyl-pyridin-2-yl)-[1,4]diazepane- | 30 |
| 1-carboxylic acid (4-methyl-thiazol-2-yl)-amide | |
| [4-(6-Chloro-benzothiazol-2-yl)-[1,4]diazepan-1-yl]- | 62 |
| (tetrahydro-pyran-4-yl)-methanone | |
| [4-(6-Chloro-benzothiazol-2-yl)-[1,4]diazepan-1-yl]-(4- | 30 |
| chloro-phenyl)-methanone | |
| [4-(3-Phenyl-[1,2,4]thiadiazol-5-yl)-[1,4]diazepan-1-yl]- | 58 |
| (tetrahydro-pyran-4-yl)-methanone | |
| (4-Chloro-phenyl)-[4-(3-phenyl-[1,2,4]thiadiazol-5-yl)- | 5 |
| [1,4]diazepan-1-yl]-methanone | |
| 4-(3-Chloro-5-trifluoromethyl-pyridin-2-yl)-[1,4]diazepane- | 4 |
| 1-carboxylic acid (5-tert-butyl-isoxazol-3-yl)-amide | |
| 4-(3-Chloro-5-trifluoromethyl-pyridin-2-yl)-[1,4]diazepane- | 27 |
| 1-carboxylic acid (4-tert-butyl-thiazol-2-yl)-amide | |
| 4-(3-Chloro-5-trifluoromethyl-pyridin-2-yl)-[1,4]diazepane- | 0.94 |
| 1-carboxylic acid (5-tert-butyl-4-methyl-thiazol-2-yl)-amide | |
| 4-(3-Chloro-5-trifluoromethyl-pyridin-2-yl)-[1,4]diazepane- | 21 |
| 1-carboxylic acid (2-tert-butylsulfanyl-ethyl)-amide | |
| 4-(3-Chloro-5-trifluoromethyl-pyridin-2-yl)-[1,4]diazepane- | 8 |
| 1-carboxylic acid (5-tert-butyl-2-methyl-2H-pyrazol-3-yl)- | |
| amide | |
| 6-Chloro-2-[4-(3-chloro-5-trifluoromethyl-pyridin-2-yl)- | 178 |
| [1,4]diazepan-1-yl]-benzothiazole | |
| 1-(3-Chloro-5-trifluoromethyl-pyridin-2-yl)-4-(3-phenyl- | 27 |
| [1,2,4]thiadiazol-5-yl)-[1,4]diazepane | |
| 4-(3,5-Dichloro-pyridin-2-yl)-[1,4]diazepane-1-carboxylic | 44 |
| acid (5-tert-butyl-isoxazol-3-yl)-amide | |
| 4-(4-Trifluoromethyl-pyrimidin-2-yl)-[1,4]diazepane-1- | 333 |
| carboxylic acid (5-tert-butyl-isoxazol-3-yl)-amide | |
| 4-(5-Trifluoromethyl-pyridin-2-yl)-[1,4]diazepane-1- | 6 |
| carboxylic acid (5-tert-butyl-isoxazol-3-yl)-amide | |
| 4-(3-Chloro-pyridin-2-yl)-[1,4]diazepane-1-carboxylic acid | 279 |
| (5-tert-butyl-isoxazol-3-yl)-amide | |
| 4-(3,5-Dichloro-pyridin-2-yl)-[1,4]diazepane-1-carboxylic | 500 |
| acid (5-tert-butyl-2-methyl-2H-pyrazol-3-yl)-amide | |
| (4-Hydroxy-cyclohexyl)-[4-(5-trifluoromethyl-benzooxazol- | 19 |
| 2-yl)-[1,4]diazepan-1-yl]-methanone | |
| 2-[4-(Tetrahydro-pyran-4-carbonyl)-[1,4]diazepan-1-yl]- | 40 |
| thiazole-5-carbonitrile | |
| [4-(5-Chloro-4-trifluoromethyl-thiazol-2-yl)-[1,4]diazepan- | 62 |
| 1-yl]-(tetrahydro-pyran-4-yl)-methanone | |
| [4-(5-Chloro-4-methyl-benzothiazol-2-yl)-[1,4]diazepan-1- | 204 |
| yl]-morpholin-4-yl-methanone | |
| [4-(7-Chloro-4-methyl-benzothiazol-2-yl)-[1,4]diazepan-1- | 2 |
| yl]-morpholin-4-yl-methanone | |
| [4-(6-Chloro-4-methyl-benzothiazol-2-yl)-[1,4]diazepan-1- | 221 |
| yl]-(tetrahydro-pyran-4-yl)-methanone | |
| [4-(7-Chloro-4-methyl-benzothiazol-2-yl)-[1,4]diazepan-1- | 114 |
| yl]-(tetrahydro-pyran-4-yl)-methanone | |
| [4-(4,7-Dimethyl-benzothiazol-2-yl)-[1,4]diazepan-1-yl]- | 445 |
| (tetrahydro-pyran-4-yl)-methanone | |
| [4-(5,7-Dimethyl-benzothiazol-2-yl)-[1,4]diazepan-1-yl]- | 53 |
| (tetrahydro-pyran-4-yl)-methanone | |
| [4-(5-Chloro-4-methyl-benzothiazol-2-yl)-[1,4]diazepan-1- | 3 |
| yl]-(tetrahydro-pyran-4-yl)-methanone | |
| 2-[4-(3-Chloro-5-trifluoromethyl-pyridin-2-yl)- | 54 |
| [1,4]diazepan-1-yl]-5-trifluoromethyl-benzooxazole | |
| [4-(5-tert-Butyl-benzooxazol-2-yl)-[1,4]diazepan-1-yl]- | 6 |
| morpholin-4-yl-methanone | |
| [4-(5-tert-Butyl-benzooxazol-2-yl)-[1,4]diazepan-1-yl]-(1,1- | 7 |
| dioxo-1λ⁶-thiomorpholin-4-yl)-methanone | |
| [4-(4-Phenyl-thiazol-2-yl)-[1,4]diazepan-1-yl]-(tetrahydro- | 217 |
| pyran-4-yl)-methanone | |
| [4-(5-Morpholin-4-ylmethyl-thiazol-2-yl)-[1,4]diazepan-1- | 14 |
| yl]-(tetrahydro-pyran-4-yl)-methanone | |
| 5-tert-Butyl-2-[4-(3-chloro-5-trifluoromethyl-pyridin-2-yl)- | 0.51 |
| [1,4]diazepan-1-yl]-benzooxazole | |
| [4-(1H-Benzoimidazol-2-yl)-[1,4]diazepan-1-yl]-(1,1- | 327 |
| dioxo-1λ⁶-thiomorpholin-4-yl)-methanone | |
| [4-(6-Methyl-benzothiazol-2-yl)-[1,4]diazepan-1-yl]- | 110 |
| (tetrahydro-pyran-4-yl)-methanone | |
| (Tetrahydro-pyran-4-yl)-[4-(5-trifluoromethyl-benzothiazol- | 35 |
| 2-yl)-[1,4]diazepan-1-yl]-methanone | |
| [4-(5-Chloro-benzothiazol-2-yl)-[1,4]diazepan-1-yl]- | 151 |
| (tetrahydro-pyran-4-yl)-methanone | |
| (4,4-Difluoro-cyclohexyl)-[4-(5-trifluoromethyl- | 10 |
| benzooxazol-2-yl)-[1,4]diazepan-1-yl]-methanone | |
| 4-(3-Chloro-5-trifluoromethyl-pyridin-2-yl)-[1,4]diazepane- | 14 |
| 1-carboxylic acid (5-sec-butyl-2-methyl-2H-pyrazol-3-yl)- | |
| amide | |
| [4-(6-Chloro-benzooxazol-2-yl)-[1,4]diazepan-1-yl]- | 180 |
| (tetrahydro-pyran-4-yl)-methanone | |
| [4-(5-Phenyl-benzooxazol-2-yl)-[1,4]diazepan-1-yl]- | 77 |
| (tetrahydro-pyran-4-yl)-methanone | |
| [4-(5-Chloro-benzooxazol-2-yl)-[1,4]diazepan-1-yl]- | 490 |
| morpholin-4-yl-methanone | |
| 4-(5-Cyano-pyridin-2-yl)-[1,4]diazepane-1-carboxylic acid | 19 |
| (5-tert-butyl-isoxazol-3-yl)-amide | |
| 4-(5-Ethyl-pyrimidin-2-yl)-[1,4]diazepane-1-carboxylic acid | 6 |
| (5-tert-butyl-isoxazol-3-yl)-amide | |
| 4-(4-Cyano-pyridin-2-yl)-[1,4]diazepane-1-carboxylic acid | 200 |
| (5-tert-butyl-isoxazol-3-yl)-amide | |
| 4-(4-Methyl-pyrimidin-2-yl)-[1,4]diazepane-1-carboxylic | 344 |
| acid (5-tert-butyl-isoxazol-3-yl)-amide | |
| [4-(5-Methyl-thieno[2,3-d]pyrimidin-4-yl)-[1,4]diazepan-1- | 45.2 |
| yl]-morpholin-4-yl-methanone | |

In all the compounds disclosed hereinabove in this application, in the event the nomenclature is in conflict with the structure, it shall be understood that the compound is defined by the structure.

The invention also relates to pharmaceutical preparations, containing as active substance one or more compounds of formula (I), or the pharmaceutically acceptable derivatives thereof, optionally combined with conventional excipients and/or carriers.

Compounds of the invention also include their isotopically-labelled forms. An isotopically-labelled form of an active agent of a combination of the present invention is identical to said active agent but for the fact that one or more atoms of said active agent have been replaced by an atom or atoms having an atomic mass or mass number different from the atomic mass or mass number of said atom which is usually found in nature. Examples of isotopes which are readily available commercially and which can be incorporated into an active agent of a combination of the present invention in accordance with well established procedures, include isotopes of hydrogen, carbon, nitrogen, oxygen, phosphorous, fluorine and chlorine, e.g., ²H, ³H, ¹³C, ¹⁴C, ¹⁵N, ¹⁸O, ¹⁷O, ³¹P, ³²P, ³⁵S, ¹⁸F, and ³⁶Cl, respectively. An active agent of a combination of the present invention, a prodrug thereof, or a pharmaceutically acceptable salt of either which contains one or more of the above-mentioned isotopes and/or other isotopes of other atoms is contemplated to be within the scope of the present invention.

The invention includes the use of any compounds of described above containing one or more asymmetric carbon atoms may occur as racemates and racemic mixtures, single enantiomers, diastereomeric mixtures and individual diastereomers. Isomers shall be defined as being enantiomers and diastereomers. All such isomeric forms of these compounds are expressly included in the present invention. Each stereogenic carbon may be in the R or S configuration, or a combination of configurations.

Some of the compounds of formula (I) can exist in more than one tautomeric form. The invention includes methods using all such tautomers.

All terms as used herein in this specification, unless otherwise stated, shall be understood in their ordinary meaning as known in the art. For example, “C₁₋₄alkoxy” is a C₁₋₄alkyl with a terminal oxygen, such as methoxy, ethoxy, propoxy, butoxy. All alkyl, alkenyl and alkynyl groups shall be understood as being branched or unbranched where structurally possible and unless otherwise specified. Other more specific definitions are as follows:

Carbocycles include hydrocarbon rings containing from three to twelve carbon atoms. These carbocycles may be either aromatic either aromatic or non-aromatic ring systems. The non-aromatic ring systems may be mono- or polyunsaturated. Preferred carbocycles include but are not limited to cyclopropyl, cyclobutyl, cyclopentyl, cyclopentenyl, cyclohexyl, cyclohexenyl, cycloheptanyl, cycloheptenyl, phenyl, indanyl, indenyl, benzocyclobutanyl, dihydronaphthyl, tetrahydronaphthyl, naphthyl, decahydronaphthyl, benzocycloheptanyl and benzocycloheptenyl. Certain terms for cycloalkyl such as cyclobutanyl and cyclobutyl shall be used interchangeably.

The term “heterocycle” refers to a stable nonaromatic 4-8 membered (but preferably, 5 or 6 membered) monocyclic or nonaromatic 8-11 membered bicyclic heterocycle radical which may be either saturated or unsaturated. Each heterocycle consists of carbon atoms and one or more, preferably from 1 to 4 heteroatoms chosen from nitrogen, oxygen and sulfur. The heterocycle may be attached by any atom of the cycle, which results in the creation of a stable structure.

The term “heteroaryl” shall be understood to mean an aromatic 5-8 membered monocyclic or 8-11 membered bicyclic ring containing 1-4 heteroatoms such as N, O and S.

Unless otherwise stated, heterocycles and heteroaryl include but are not limited to, for example benzoxazolyl, benzothiazolyl, benzimidazolyl, tetrahydropyranyl, dioxanyl, tetrahydrofuranyl, oxazolyl, isoxazolyl, thiazolyl, pyrazolyl, pyrrolyl, imidazolyl, thienyl, thiadiazolyl, thiomorpholinyl, 1,1-Dioxo-1λ⁶-thiomorpholinyl, morpholinyl, pyridinyl, pyrimidinyl, pyridazinyl, pyrazinyl, triazinyl, pyrrolidinyl, piperidinyl, piperazinyl, purinyl, quinolinyl, Dihydro-2H-quinolinyl, isoquinolinyl, quinazolinyl, indazolyl, thieno[2,3-d]pyrimidinyl, indolyl, isoindolyl, benzofuranyl, benzopyranyl and benzodioxolyl.

The term “heteroatom” as used herein shall be understood to mean atoms other than carbon such as O, N, S and P.

In all alkyl groups or carbon chains one or more carbon atoms can be optionally replaced by heteroatoms: O, S or N, it shall be understood that if N is not substituted then it is NH, it shall also be understood that the heteroatoms may replace either terminal carbon atoms or internal carbon atoms within a branched or unbranched carbon chain. Such groups can be substituted as herein above described by groups such as oxo to result in definitions such as but not limited to: alkoxycarbonyl, acyl, amido and thioxo.

The term “aryl” as used herein shall be understood to mean aromatic carbocycle or heteroaryl as defined herein. Each aryl or heteroaryl unless otherwise specified includes it's partially or fully hydrogenated derivative. For example, quinolinyl may include decahydroquinolinyl and tetrahydroquinolinyl, naphthyl may include its hydrogenated derivatives such as tetrahydronaphthyl. Other partially or fully hydrogenated derivatives of the aryl and heteroaryl compounds described herein will be apparent to one of ordinary skill in the art.

As used herein, “nitrogen” and “sulfur” include any oxidized form of nitrogen and sulfur and the quaternized form of any basic nitrogen. For example, for an —S—C₁₋₆ alkyl radical, unless otherwise specified, this shall be understood to include —S(O)—C₁₋₆ alkyl and —S(O)₂—C₁₋₆ alkyl.

The term “halogen” as used in the present specification shall be understood to mean bromine, chlorine, fluorine or iodine, preferably fluorine. The definitions “partially or fully halogenated”; partially or fully fluorinated; “substituted by one or more halogen atoms”, includes for example, mono, di or tri halo derivatives on one or more carbon atoms. For alkyl, a nonlimiting example would be —CH₂CHF₂, —CF₃ etc.

The compounds of the invention are only those which are contemplated to be ‘chemically stable’ as will be appreciated by those skilled in the art. For example, a compound which would have a ‘dangling valency’, or a ‘carbanion’ are not compounds contemplated by the inventive methods disclosed herein.

The invention includes pharmaceutically acceptable derivatives of compounds of formula (I). A “pharmaceutically acceptable derivative” refers to any pharmaceutically acceptable salt or ester, or any other compound which, upon administration to a patient, is capable of providing (directly or indirectly) a compound useful for the invention, or a pharmacologically active metabolite or pharmacologically active residue thereof. A pharmacologically active metabolite shall be understood to mean any compound of the invention capable of being metabolized enzymatically or chemically. This includes, for example, hydroxylated or oxidized derivative compounds of the formula (I).

Pharmaceutically acceptable salts include those derived from pharmaceutically acceptable inorganic and organic acids and bases. Examples of suitable acids include hydrochloric, hydrobromic, sulfuric, nitric, perchloric, fumaric, maleic, phosphoric, glycolic, lactic, salicylic, succinic, toluene-p-sulfuric, tartaric, acetic, citric, methanesulfonic, formic, benzoic, malonic, naphthalene-2-sulfuric and benzenesulfonic acids. Other acids, such as oxalic acid, while not themselves pharmaceutically acceptable, may be employed in the preparation of salts useful as intermediates in obtaining the compounds and their pharmaceutically acceptable acid addition salts. Salts derived from appropriate bases include alkali metal (e.g., sodium), alkaline earth metal (e.g., magnesium), ammonium and N—(C₁-C₄ alkyl)₄⁺ salts.

In addition, within the scope of the invention is use of prodrugs of compounds of the formula (I). Prodrugs include those compounds that, upon simple chemical transformation, are modified to produce compounds of the invention. Simple chemical transformations include hydrolysis, oxidation and reduction. Specifically, when a prodrug is administered to a patient, the prodrug may be transformed into a compound disclosed hereinabove, thereby imparting the desired pharmacological effect.

The compounds of formula I may be made using the general synthetic methods described below, which also constitute part of the invention.

General Synthetic Methods

The invention also provides processes for making compounds of Formula (I). In all methods, unless specified otherwise, G, L and Ar₂ in the formulas below shall have the meaning of G, L and Ar₂ in Formula (I) of the invention described herein above.

Optimum reaction conditions and reaction times may vary depending on the particular reactants used. Unless otherwise specified, solvents, temperatures, pressures, and other reaction conditions may be readily selected by one of ordinary skill in the art. Specific procedures are provided in the Synthetic Examples section. Typically, reaction progress may be monitored by thin layer chromatography (TLC), if desired, and intermediates and products may be purified by chromatography on silica gel and/or by recrystallization.

The examples which follow are illustrative and, as recognized by one skilled in the art, particular reagents or conditions could be modified as needed for individual compounds without undue experimentation. Starting materials and intermediates used, in the methods below, are either commercially available or easily prepared from commercially available materials by those skilled in the art.

Further modification of the initial product of formula (I) by methods known in the art and illustrated in the Examples below, may be used to prepare additional compounds of this invention.

Compounds of formula (I) wherein L is a bond, —C(O) or —CH₂—C(O)—, may be synthesized by the methods outlined in Schemes 1 and 2.

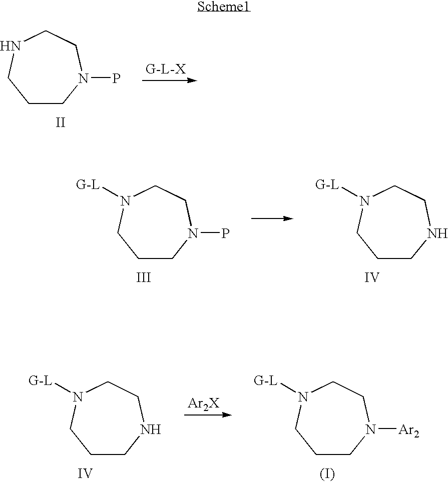

As illustrated in Scheme 1, reaction of a protected 1,4 diazepane of formula II with an acid chloride of formula G-L-X, wherein X=Cl, in a suitable solvent, in the presence of a suitable base, provides a compound of formula III. P is a suitable protecting group such as BOC. Alternatively, the 1,4 diazepane of formula II may also be coupled with the corresponding acid under standard coupling conditions, to provide a compound of formula III. Standard peptide coupling reactions known in the art (see for example M. Bodanszky, 1984, The Practice of Peptide Synthesis, Springer-Verlag) may be employed in these syntheses. An example of suitable coupling conditions is treatment of a solution of the carboxylic acid in a suitable solvent such as DMF with EDC, HOBT, and a base such as diisopropylethylamine, followed by the desired amine.

Deprotection of the compound of formula III under standard conditions, in a suitable solvent, using a reagent such as trifluoroacetic acid, provides a free base of formula IV. Reaction of the compound of formula IV with Ar₂X, wherein X=Cl or Br, in a suitable solvent, in the presence of a suitable base, provides a compound of formula (I).

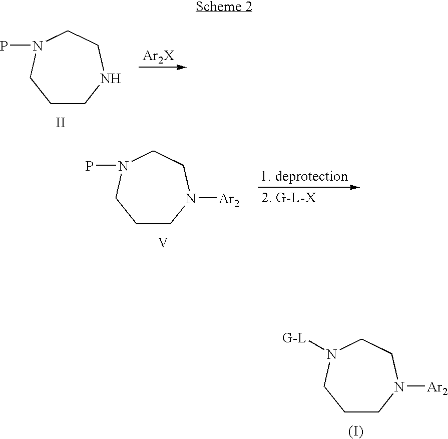

As shown in Scheme 2, reaction of a protected 1,4 diazepane of formula II with Ar₂X, wherein X=Cl or Br, in a suitable solvent, in the presence of a suitable base, provides a compound of formula V. As in Scheme 1, deprotection of the compound of formula V followed by reaction of the free amine with the acid chloride G-L-X or the corresponding acid, provides a compound of formula (I).

Further modification of the initial product of formula (I) by methods known in the art and illustrated in the Examples below, may be used to prepare additional compounds of this invention.

Compounds of formula (I) wherein L is —NH—C(O)—, may be synthesized by the methods outlined in Schemes 3 and 4.

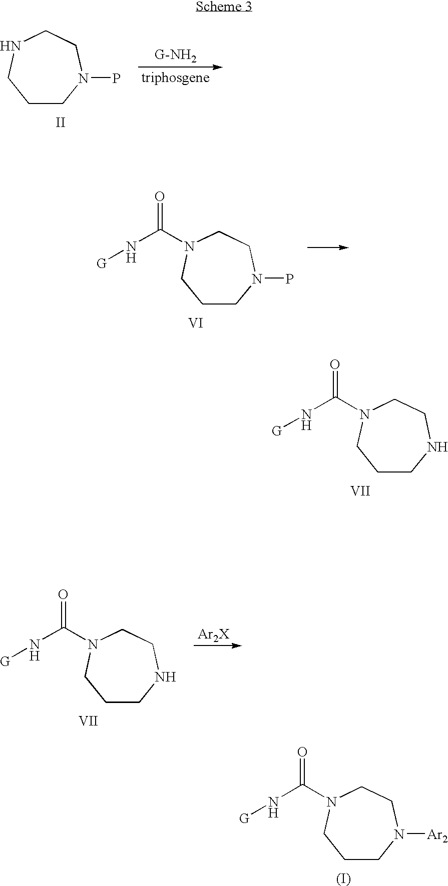

As outlined in Scheme 3, reaction of a protected 1,4 diazepane of formula II with an amine G-NH₂ and triphosgene, in a suitable solvent, in the presence of a suitable base, provides a compound of formula VI. P is a suitable protecting group such as BOC.

Deprotection of the compound of formula VI under standard conditions, in a suitable solvent, using a reagent such as trifluoroacetic acid, provides a free base of formula VII.

Reaction of the compound of formula VII with Ar₂X, wherein X=Cl or Br, in a suitable solvent, in the presence of a suitable base, provides a compound of formula (I).

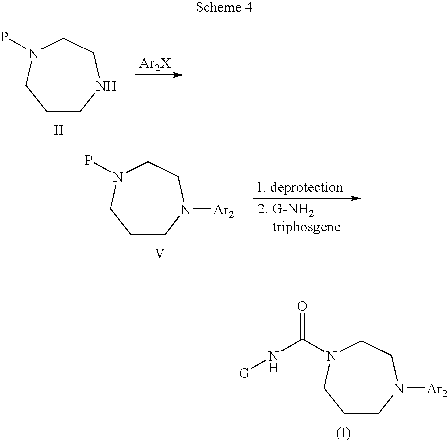

As shown in Scheme 4, reaction of a protected 1,4 diazepane of formula II with Ar₂X, wherein X=Cl or Br, in a suitable solvent, in the presence of a suitable base, provides a compound of formula V. As in Scheme 3, deprotection of the compound of formula V followed by reaction of the diazepane with the amine G-NH₂ and triphosgene, provides a compound of formula (I). Further modification of the initial product of formula (I) by methods known in the art and illustrated in the Examples below, may be used to prepare additional compounds of this invention.

### EXAMPLES

The manner in which the compounds of the invention can be made will be further understood by way of the following Examples.

### Method 1: Synthesis of 1-[4-(5-tert-Butyl-benzoxazol-2-yl)-perhydro-1,4-diazepin-1-yl]-2-cyclopentyl-ethanone

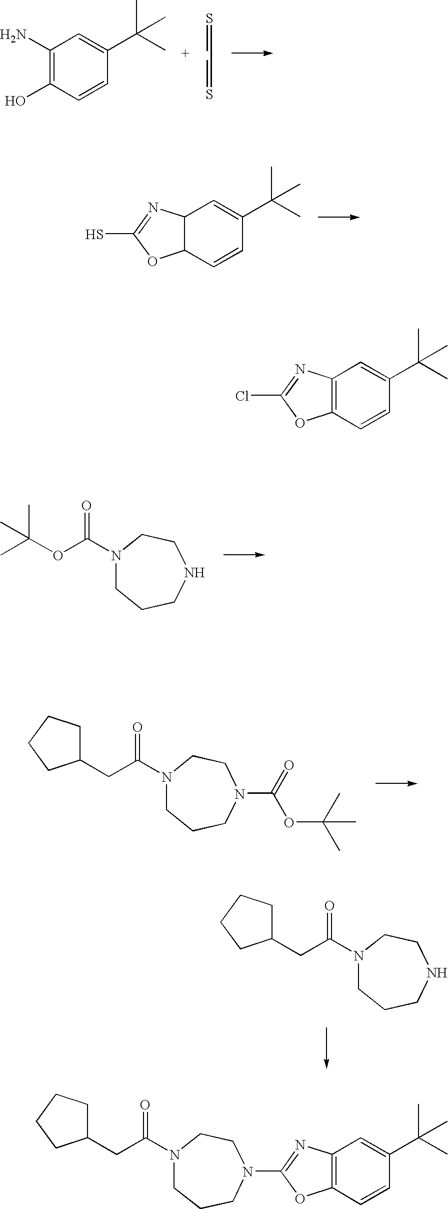

### Step 1: Synthesis of 5-tert-Butyl-benzoxazole-2-thiol

Prepared as described by adaptation of the following reference:
Katz et al. J. Org. Chem. 1954, 19, 758-766

A solution of potassium methyl xanthate is prepared by dissolving 44 mg (0.78 mmol) of potassiuim hydroxide in 0.7 mL of methanol and 0.12 mL of water. 0.04 mL (0.67 mmol) of carbon disulfide is added with stirring. 100 mg (0.61 mmol) of 2-amino-4-tert-butylphenol is added and the vial is sealed and heated to reflux for 18 hours. The reaction is cooled to room temperature and the mixture is dried under nitrogen to remove solvents. Brown solid is obtained and it is dispersed in 1 mL dichloromethane and 0.12 mL acetic acid is added. It is then heated to its boiling point. Water is added and it is extracted with dichloromethane twice. The organics are combined and washed with brine, dried (Na₂SO₄), filtered and concentrated. The residue is purified by flash chromatography using ethyl acetate/hexanes as eluent mixtures and 110 mg of a dark brown oil is obtained.

According to this procedure, the following 2-mercaptobenzoxazole are synthesized:

TABLE 1

| | | YIELD | m/z |
|---|---|---|---|
| MOLSTRUCTURE | NAME | [%] | [M + H⁺] |
| 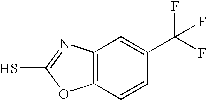 | 5- trifluoromethyl- benzoxazole- 2-thiol | 34 | N/A |

### Step 2: Synthesis of 5-tert-butyl-2-chloro-benzoxazole

Prepared as described by adaptation of the following reference:
Lazer et al. J. Med. Chem. 1994, 37, 913-923

To a suspension of 106 mg (0.51 mmol) of 5-tert-butyl-benzoxazole-2-thiol in 0.42 mL (4.53 mmol) of phosphorous (III) oxychloride at room temperature is added 127 mg (0.61 mmol) of phosphorous (V) chloride along with 1 mL of dichloromethane. After 1 h of stirring at room temperature, the reaction mixture is concentrated to remove excess phosphorous (III) oxychloride, and the residue is diluted with dichloromethane and washed with sodium carbonate aqueous solution. The aqueous phase is extracted with dichloromethane and the combined dichloromethane extracts are washed with brine, dried (MgSO₄), filtered and concentrated. Flash chromatography on silica gel starting with hexanes followed by 10% ethyl acetate/hexanes provides 69 mg of 5-tert-butyl-2-chloro-benzoxazole as a brown oil.

According to this procedure the following 2-chlorobenzoxazoles are synthesized:

TABLE 2

| | | YIELD | m/z |
|---|---|---|---|
| MOLSTRUCTURE | NAME | [%] | [M + H⁺] |
| 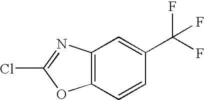 | 5- trifluoromethyl- 2-chloro- benzoxazole | 65 | N/A |

### Step 3: Synthesis of 4-(2-Cyclopentyl-acetyl)-perhydro-1,4-diazepine-1-carboxylic acid tert-butyl ester

To a solution of 2.2 mL (17.7 mmol) of cyclopentyl-acetic acid and 2.03 g (21.1 mmol) of 1-hydroxybenzotriazole in 20 mL dimethylformamide is added 4.05 g (21.1 mmol) of N-(3-dimethylaminopropyl)-N′-ethylcarbodiimide. After 15 min of stiffing at room temperature 3 mL (15.4 mmol) of tert-butyl-1-homopiperazinecarboxylate is added, then 75 mg (0.62 mmol) of dimethylaminopyridine is added. The reaction mixture is left stirring for 18 hours at room temperature. Water is added and it is extracted with ethyl acetate twice. The organics are combined and washed with water, saturated sodium bicarbonate aqueous solution, saturated, ammonium chloride aqueous solution and brine, dried (Na₂SO₄), filtered and concentrated. The residue is purified by flash chromatography using 50% ethyl acetate/hexanes then switched to 5% methanol/dichloromethane as eluent mixtures. 4.05 g slightly yellow oil is obtained as the product.

According to this procedure, the following 1,4-diazepine-1-carboxylic acid tert-butyl esters are synthesized.

TABLE 3

| | | YIELD | m/z |
|---|---|---|---|
| MOLSTRUCTURE | NAME | [%] | [M + H⁺] |
| 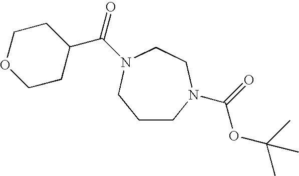 | 4-(Tetrahydro-pyran-4-carbonyl)- [1,4]diazepane-1-carboxylic acid tert- butyl ester | 58 | 313 |
| 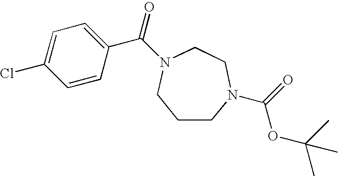 | 4-(4-Chloro-benzoyl)-[1,4]diazepane- 1-carboxylic acid tert-butyl ester | 70 | 339 |
| 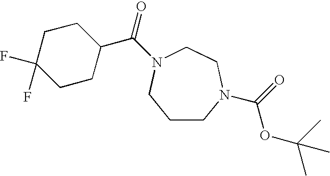 | 4-(4,4-Difluoro- cyclohexanecarbonyl)-[1,4]diazepane- 1-carboxylic acid tert-butyl ester | 92 | 347 |

### Step 4: Synthesis of 2-Cyclopentyl-1-perhydro-1,4-diazepin-1-yl-ethanone; hydrochloride

4.05 g (13 mmol) of 4-(2-cyclopentyl-acetyl)-perhydro-1,4-diazepine-1-carboxylic acid tert-butyl ester is dissolved in 50 mL methanol and treated at room temperature with 32.5 mL (130 mmol) of 4N solution of hydrogen chloride in 1,4-dioxane. The reaction mixture is stirred at room temperature for 3.5 hours. The mixture is concentrated in vacuo to afford 3.449 g of 2-cyclopentyl-1-perhydro-1,4-diazepin-1-yl-ethanone; hydrochloride.

According to this procedure, the following 1,4-diazepine-1-carboxylic acid tert-butyl ester hydrochlorides were synthesized.

TABLE 4

| | | YIELD | m/z |
|---|---|---|---|
| MOLSTRUCTURE | NAME | [%] | [M + H⁺] |
| 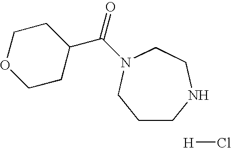 | [1,4]Diazepan-1-yl-(tetrahydro-pyran- 4-yl)-methanone; hydrochloride | 95 | 213 |
| 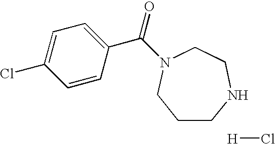 | (4-Chloro-phenyl)-[1,4]diazepan-1-yl- methanone; hydrochloride | 80 | 239/241 |
| 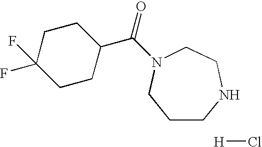 | [1,4]Diazepan-1-yl-(4,4-difluoro- cyclohexyl)-methanone; hydrochloride | 100 | 247 |

### Step 5: Synthesis of 1-[4-(5-tert-Butyl-benzoxazol-2-yl)-perhydro-1,4-diazepin-1-yl]-2-cyclopentyl-ethanone

Prepared as described by adaptation of the following reference:
Lazer et al. J. Med. Chem. 1994, 37, 913-923

A mixture of 69 mg (0.33 mmol) of 5-tert-butyl-2-chloro-benzoxazole, 80 mg (0.33 mmol) of the amine and 0.14 mL (0.78 mmol) of N,N-diisopropylethylamine in 2 mL of dichloromethane is heated to reflux for 18 hours. The reaction mixture is directly purified by flash chromatography using 2.5% methanol/dichloromethane as eluent mixtures. 104 mg product is obtained as a yellow oil.

Examples in Table 12 Method 1 are prepared according to a similar procedure.

### Method 2: Synthesis of [4-(6-Methyl-benzothiazol-2-yl)-[1,4]diazepan-1-yl]-(tetrahydro-pyran-4-yl)-methanone

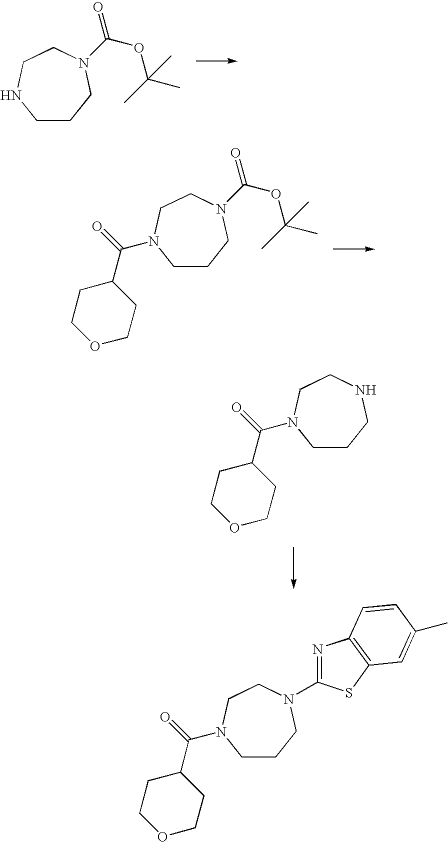

### Step 1: Synthesis of 4-(Tetrahydro-pyran-4-carbonyl)-[1,4]diazepane-1-carboxylic acid tert-butyl ester

To a solution of 5 g (38.5 mmol) of tetrahydro-pyran-4-carboxylic acid in anhydrous dichloromethane (50 mL) at 0° C. is added 7 mL (96.2 mmol) of thionyl chloride dropwise. The reaction mixture is stirred at room temperature for 23 h. The reaction is concentrated under reduced pressure, dissolved in anhydrous tetrahydrofuran (50 mL) and cooled to 0° C. To this cooled solution 7.17 mL (36.9 mmol) of [1,4]diazepane-1-carboxylic acid tert-butyl ester and 25.5 mL (231 mmol) of triethylamine in anhydrous tetrahydrofuran (50 mL) are added dropwise over 15 min. The reaction mixture is stirred at room temperature for 18 h. The reaction is concentrated under reduced pressure and the residue is dissolved in dichloromethane (100 mL). The organic layer is washed with saturated aqueous ammonium chloride solution (100 mL), saturated aqueous sodium bicarbonate solution (100 mL) and brine (100 mL). The organic layer is dried (Na₂SO₄), filtered and the filtrate is concentrated under reduced pressure to afford 8.58 g of 4-(tetrahydro-pyran-4-carbonyl)-[1,4]diazepane-1-carboxylic acid tert-butyl ester. ES MS m/z 313 (M+H).

### Step 2: Synthesis of [1,4]Diazepan-1-yl-(tetrahydro-pyran-4-yl)-methanone

To a solution of 8.58 g (27.5 mmol) 4-(tetrahydro-pyran-4-carbonyl)-[1,4]-diazepane-1-carboxylic acid tert-butyl ester in dichloromethane (86 mL) is added trifluoroacetic acid (21.5 mL). The reaction mixture is stirred at room temperature for 20 h. The reaction is concentrated under reduced pressure to constant weight to afford 14.8 g of [1,4]diazepan-1-yl-(tetrahydro-pyran-4-yl)-methanone as the trifluoroacetic acid salt (three equivalents by mass). ES MS m/z 213 (M+H).

### Step 3: Synthesis of [4-(6-Methyl-benzothiazol-2-yl)-[1,4]diazepan-1-yl]-(tetrahydro-pyran-4-yl)-methanone

To a solution of 0.25 g (0.451 mmol) of [1,4]diazepan-1-yl-(tetrahydro-pyran-4-yl)-methanone (three trifluoroacetic acid equivalents, prepared according to step 1-2, Method 2) in toluene-isopropanol (4:1, 5 mL) are added 82.8 mg (0.451 mmol) of 2-chloro-6-methyl-1,3-benzothiazole, followed by 0.392 mL (2.26 mmol) of N,N-diisopropylethylamine. The reaction mixture is heated in a sealed tube to 150° C. for 22 h. The reaction is cooled to room temperature, concentrated under reduced pressure and the residue dissolved in dichloromethane (5 mL). The organic layer is washed with water (5 mL), dried (Na₂SO₄), filtered and the filtrate is concentrated under reduced pressure. The crude product is purified by column chromatography (silica, eluent: ethyl acetate) to afford 90.5 mg of [4-(6-methyl-benzothiazol-2-yl)-[1,4]diazepan-1-yl]-(tetrahydro-pyran-4-yl)-methanone. ES MS m/z 360 (M+H).

Examples in Table 12 Method 2 are prepared according to a similar procedure.

### Method 3: Synthesis of [4-(4-Methyl-thiazol-2-yl)-[1,4]diazepan-1-yl]-(tetrahydro-pyran-4-yl)-methanone

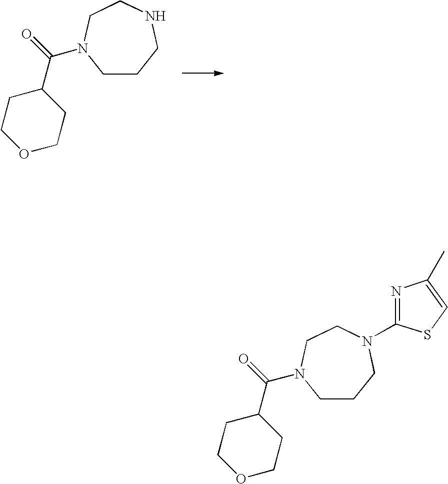

To a solution of 0.25 g (0.451 mmol) [1,4]diazepan-1-yl-(tetrahydro-pyran-4-yl)-methanone (three trifluoroacetic acid equivalents, prepared according to step 1-2, Method 2) in 1-methyl-2-pyrrolidone (3 mL) are added sequentially 0.187 g (1.35 mmol) of potassium carbonate, 85.9 mg (0.451 mmol) of copper iodide and 60.2 mg (0.451 mmol) of 2-chloro-4-methyl-thiazole. The reaction mixture is heated in a sealed tube at 190° C. for 1 h in a CEM Discover microwave reactor. The reaction is cooled to room temperature and ethyl acetate (5 mL) is added. The organic layer is washed with water (2×5 mL), dried (Na₂SO₄), filtered and the filtrate is concentrated under reduced pressure. The crude product is purified by preparative HPLC (column: Phenomenex Gemini C18 AXIA 5u 100×21.2 mm, eluent: 2 mM NH₄HCO₃:95:5 MeCN:2 mM NH₄HCO₃) to afford 11.0 mg of [4-(4-methyl-thiazol-2-yl)-[1,4]diazepan-1-yl]-(tetrahydro-pyran-4-yl)-methanone. ES MS m/z 310 (M+H).

Examples in Table 12 Method 3 are prepared according to a similar procedure.

### Method 4: Synthesis of [4-(5-Chloro-benzothiazol-2-yl)-[1,4]diazepan-1-yl]-(tetrahydro-pyran-4-yl)-methanone

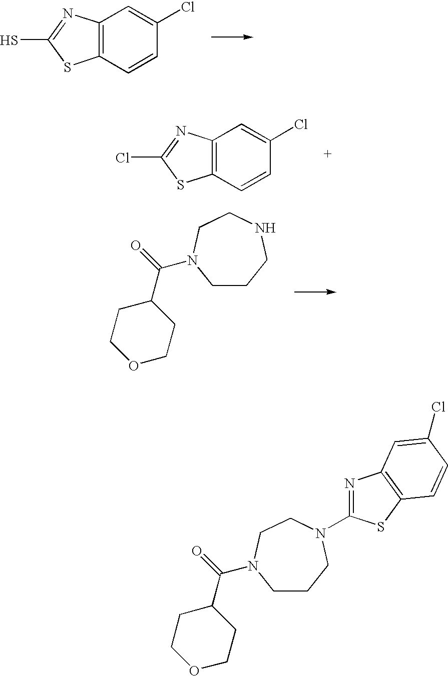

### Step 1: 2,5-Dichloro-benzothiazole

Prepared as described by adaptation of the following reference:
Moon, N. S., U.S. Pat. No. 2,469,697

To 0.1 g (0.496 mmol) of 5-chloro-benzothiazole-2-thiol are added 0.349 mL (4.31 mmol) of SO₂Cl₂. The reaction mixture is shaken at room temperature for 1.5 h. Water (2 mL) and dichloromethane (5 mL) are added. The organic layer is washed with water (3 mL), dried (Na₂SO₄), filtered and the filtrate is concentrated under reduced pressure to afford 81.2 mg of 2,5-dichloro-benzothiazole. ES MS no ionization observed, intermediate characterized by ¹H NMR (400 MHz, CHLOROFORM-d) δ ppm 7.42 (1H, dd, J=8.68, 2.08 Hz), 7.71 (1H, d, J=8.80 Hz), 7.96 (1H, d, J=2.20 Hz).

According to this method the following 2-chlorothiazole is synthesized.

TABLE 5

| | | | 1H NMR (400 MHz, |
|---|---|---|---|
| Structure | Name | Yield [%] | CHLOROFORM-d) |
| 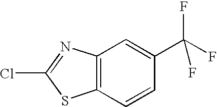 | 2-Chloro-5- trifluoromethyl- benzothiazole | 71 | δ ppm 7.68 (1 H, dd, J = 8.56, 1.22 Hz), 7.93 (1 H, d, J = 8.31 Hz), 8.23 (1 H, d) |

### Step 2: Synthesis of [4-(5-Chloro-benzothiazol-2-yl)-[1,4]diazepan-1-yl]-(tetrahydro-pyran-4-yl)-methanone

To a solution of 0.234 g (0.398 mmol) of [1,4]diazepan-1-yl-(tetrahydro-pyran-4-yl)-methanone (three trifluoroacetic acid equivalents, prepared according to step 1-2, Method 2) in toluene-isopropanol (4:1, 5 mL) are added 81.2 mg (0.398 mmol) of 2,5-dichloro-benzothiazole, followed by 0.348 mL (2.11 mmol) of N,N-diisopropylethylamine. The reaction mixture is heated in a sealed tube to 150° C. for 17 h. The reaction is cooled to room temperature, concentrated under reduced pressure and the residue is dissolved in dichloromethane (5 mL). The organic layer is washed with water (3 mL), dried (Na₂SO₄), filtered and the filtrate is concentrated under reduced pressure. The crude product is purified by column chromatography (silica, eluent: ethyl acetate) to afford 30.5 mg of [4-(5-chloro-benzothiazol-2-yl)-[1,4]diazepan-1-yl]-(tetrahydro-pyran-4-yl)-methanone. ES MS m/z 380, 382 (M+H).

Examples in Table 12 Method 4 are prepared according to a similar procedure.

### Method 5: Synthesis of (1,1-Dioxo-1λ⁶-thiomorpholin-4-yl)-[4-(5-trifluoromethyl-benzooxazol-2-yl)-[1,4]diazepan-1-yl]-methanone

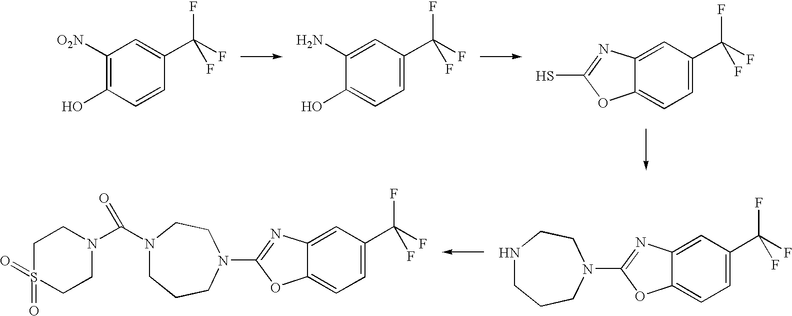

### Step 1: Synthesis of 2-Amino-4-(trifluoromethyl)phenol

To a solution of 5 g (24.1 mmol) of 2-nitro-4-(trifluoromethyl)phenol in ethanol (100 mL) is added 0.5 g of palladium on charcoal (10 wt %). The reaction mixture is reduced under hydrogen atmosphere at room temperature for 16 h with stiffing. The catalyst is separated by filtration through Celite®. The filtrate is concentrated under reduced pressure to afford 4.3 g of 2-amino-4-(trifluoromethyl)phenol. ES MS m/z=178 (M+H).

### Step 2: Synthesis of 5-Trifluoromethyl-benzooxazole-2-thiol

To a solution of 4.2 g (23.7 mmol) of 2-amino-4-(trifluoromethyl)phenol in ethanol (100 mL) is added 3.8 g (23.7 mmol) of potassium ethyl xanthate. The reaction mixture is refluxed for 16 h and then concentrated under reduced pressure. Water (50 ml) is added and the pH adjusted to ˜4-5 with acetic acid. The solids are separated by filtration and the filter cake washed with ethanol (40 mL). The filtrate is concentrated under reduced pressure. The crude product is purified by column chromatography (silica, eluent: dichloromethane, 2% ethyl acetate) to afford 1.95 g of 5-trifluoromethyl-benzooxazole-2-thiol. ES MS m/z=261 (M+H+MeCN)

According to this method the following benzoxazolethiols are synthesized.

TABLE 6

| | | Yield | m/z |
|---|---|---|---|
| Structure | Name | (%) | [M + H]+ |
| 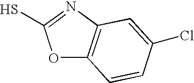 | 5-Chloro-benzooxazole-2-thiol | 64 | 184.2 and 186.0 |
| 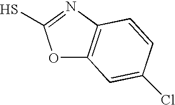 | 6-Chloro-benzooxazole-2-thiol | 95 | 184.2 and 186.2 |
| 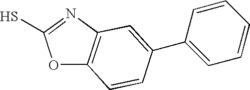 | 5-Phenyl-benzooxazole-2-thiol | 86 | 228.2 |
| 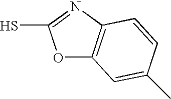 | 5-Methyl-benzooxazole-2-thiol | 92 | 166.3 |
| 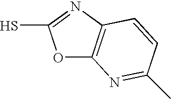 | 5-Methyl-oxazolo[5,4-b]pyridine-2-thiol | 51 | 167.3 |

### Step 3: Synthesis of 2-[1,4]Diazepan-1-yl-5-trifluoromethyl-benzooxazole

To a suspension of 219 mg (1 mmol) of 5-trifluoromethyl-benzooxazole-2-thiol in toluene (5 ml) is added 200 mg (2 mmol) of [1,4]diazepane. The reaction mixture is heated to reflux overnight. After cooling to room temperature, the mixture is concentrated under reduced pressure and the residue is purified by column chromatography (silica, eluent: dichloromethane, 10% methanol) to afford 92 mg of 2-[1,4]diazepan-1-yl-5-trifluoromethyl-benzooxazole. ES MS m/z 286 (M+H).

According to this method the following benzoxazoles are synthesized

TABLE 7

| | | Yield | m/z |
|---|---|---|---|
| Structure | Name | (%) | [M + H]+ |
| 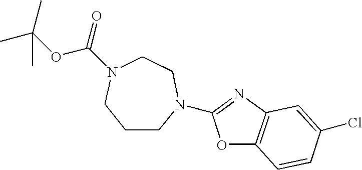 | 4-(5-Chloro-benzooxazol-2-yl)- [1,4]diazepane-1-carboxylic acid tert- butyl ester | 54 | 352.3 and 354.2 |
| 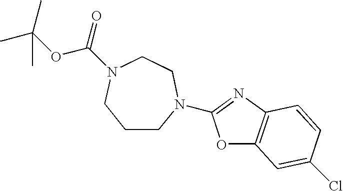 | 4-(6-Chloro-benzooxazol-2-yl)- [1,4]diazepane-1-carboxylic acid tert- butyl ester | 88 | 352.3 and 354.3 |
| 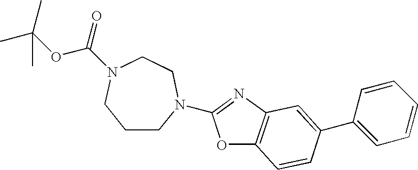 | 4-(5-Phenyl-benzooxazo1-2-yl)- [1,4]diazepane-1-carboxylic acid tert- butyl ester | 65 | 394.2 |
| 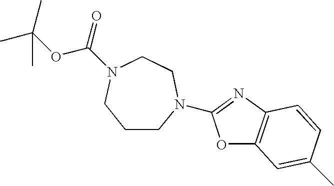 | 4-(5-Methyl-benzooxazo1-2-yl)- [1,4]diazepane-1-carboxylic acid tert- butyl ester | 40 | 332.3 |
| 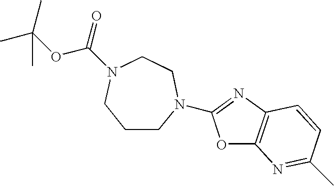 | 4-(5-Methyl-oxazolo[5,4-b]pyridin-2-yl)- [1,4]diazepane-1-carboxylic acid tert- butyl ester | 45 | 333.3 |

### Step 4: Synthesis of (1,1-Dioxo-1λ⁶-thiomorpholin-4-yl)-[4-(5-trifluoromethyl-benzooxazol-2-yl)-[1,4]diazepan-1-yl]-methanone

To a solution of 88 mg (0.31 mmol) of 2-[1,4]diazepan-1-yl-5-trifluoromethyl-benzooxazole in THF (5 ml) is added 48 mg (0.37 mmol) of N,N-diisopropylethylamine and 61 mg (0.31 mmol) of 1,1-dioxo-1λ⁶-thiomorpholine-4-carbonyl chloride. The reaction mixture is stirred for 16 h and then concentrated under reduced pressure. The residue is dissolved in dichloromethane (5 mL) and washed with water (4 mL). The organic layer is dried (Na₂SO₄), filtered and the filtrate is concentrated under reduced pressure. The crude product is purified by column chromatography (silica, eluent: ethyl acetate) to afford 91 mg, of (1,1-dioxo-1λ⁶-thiomorpholin-4-yl)-[4-(5-trifluoromethyl-benzooxazol-2-yl)-[1,4]diazepan-1-yl]-methanone. ES MS m/z=447 (M+H).

Examples in Table 12 Method 5 are prepared according to a similar procedure.

### Method 6: Synthesis of (4-Hydroxy-cyclohexyl)-[4-(5-trifluoromethyl-benzooxazol-2-yl)-[1,4]diazepan-1-yl]-methanone

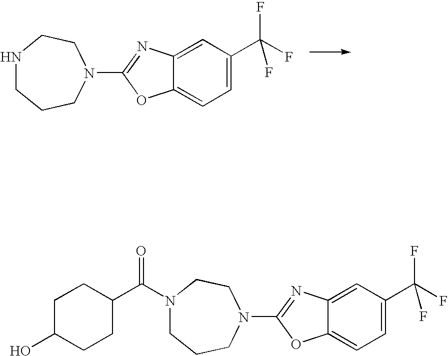

To a solution of 90 mg (0.32 mmol) of 2-[1,4]diazepan-1-yl-5-trifluoromethyl-benzooxazole (prepared according to step 1-3, Method 5) in dichloromethane (4 ml) and dimethylformamide (1 ml) are added 55 mg (0.38 mmol) of 4-hydroxy-cyclohexanecarboxylic acid and 396 mg (0.63 mmol) of polystyrene supported N,N′-dicyclohexylcarbodiimide. The reaction mixture is shaken for 2 days and filtered. The polymer is washed with dichloromethane (4 mL) and methanol (4 mL). The filtrate is concentrated under reduced pressure. The crude product is purified by column chromatography (silica, eluent: ethyl acetate, 5% methanol) to afford 58 mg of (4-hydroxy-cyclohexyl)-[4-(5-trifluoromethyl-benzooxazol-2-yl)-[1,4]diazepan-1-yl]-methanone. ES MS m/z=412 (M+H)

Examples in Table 12 Method 6 are prepared according to a similar procedure.

### Method 7: Synthesis of 2-[4-(3-Chloro-5-trifluoromethyl-pyridin-2-yl)-[1,4]diazepan-1-yl]-5-trifluoromethyl-benzooxazole

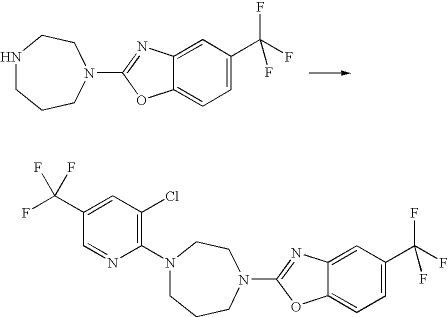

To a solution of 100 mg (0.35 mmol) of 2-[1,4]diazepan-1-yl-5-trifluoromethyl-benzooxazole (prepared according to step 1-3, Method 5) in ethoxyethanol (2 ml) are added 76 mg (0.35 mmol) of 2,3-dichloro-5-trifluoromethylpyridine and 136 mg (1.05 mmol) of N,N-diisopropylethylamine. The reaction mixture is heated in a sealed tube to 100° C. for 16 h. The reaction mixture is diluted with dichloromethane (8 mL) and washed with brine (5 mL), dried (Na₂SO₄) and filtered. The filtrate is concentrated under reduced pressure. The crude product is purified by column chromatography (silica, eluent: dichloromethane, 50% ethyl acetate) to afford 41 mg of 2-[4-(3-chloro-5-trifluoromethyl-pyridin-2-yl)-[1,4]diazepan-1-yl]-5-trifluoromethyl-benzooxazole. ES MS m/z=465, 467 (M+H).

Examples in Table 12 Method 7 are prepared according to a similar procedure.

### Method 8: Synthesis of (4,4-Difluoro-cyclohexyl)-[4-(5-trifluoromethyl-benzooxazol-2-yl)-[1,4]diazepan-1-yl]-methanone

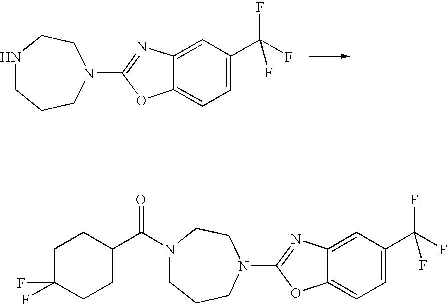

To a solution of 100 mg (0.35 mmol) of 2-[1,4]diazepan-1-yl-5-trifluoromethyl-benzooxazole (prepared according to step 1-3, Method 5) in dichloromethane (5 ml) is added 69 mg (0.42 mmol) of 4,4-difluorocyclohexanecarboxylic acid and 1.1 g (1.75 mmol) of polystyrene supported N,N′-dicyclohexylcarbodiimide. The reaction mixture is shaken for 6 days. The polymer is separated by filtration and rinsed with dichloromethane (4 mL) and methanol (4 mL). The filtrate is concentrated under reduced pressure. The residue is purified by preparative HPLC (column: Phenomenex Gemini C18 AXIA 5u 100×21.2 mm, eluent: 2 mM NH₄HCO₃: 95:5 MeCN:2 mM NH₄HCO₃) to afford 43 mg of (4,4-difluoro-cyclohexyl)-[4-(5-trifluoromethyl-benzooxazol-2-yl)-[1,4]diazepan-1-yl]-methanone. ES MS m/z=432 (M+H)

Examples in Table 12 Method 8 are prepared according to a similar procedure.

### Method 9: Synthesis of [4-(5-tert-Butyl-benzooxazol-2-yl)-[1,4]diazepan-1-]-morpholin-4-yl-methanone

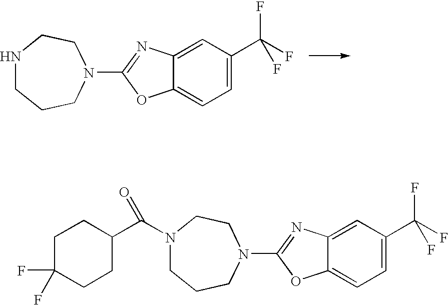

### Step 1: Synthesis of 5-tert-Butyl-benzooxazole-2-thiol

To a solution of 5 g (30.3 mmol) of 2-amino-4-tert-butylphenol in ethanol (50 mL) is added 4.85 g (30.3 mmol) of potassium ethyl xanthate. The reaction mixture is refluxed for 16 h and then concentrated under reduced pressure. Water (50 ml) is added and the pH is adjusted to 4-5 with acetic acid. The suspension is filtered and the filter cake is rinsed with ethanol (20 mL). The filtrate is concentrated under reduced pressure. The residue is purified by column chromatography (silica, eluent: dichloromethane) to afford 1.1 g of 5-tert-butyl-benzooxazole-2-thiol. ES MS m/z=208 (M+H).

### Step 2: Synthesis of 5-tert-Butyl-2-[1,4]diazepan-1-yl-benzooxazole

To a suspension of 250 mg (1.21 mmol) of 5-tert-butyl-benzooxazole-2-thiol in toluene (5 ml) is added 241 mg (2.41 mmol) of [1,4]diazepane. The reaction mixture is heated to reflux for 18 h. After cooling to room temperature, the mixture is concentrated under reduced pressure and the residue is purified by column chromatography (silica, eluent: ethyl acetate) to afford 233 mg of 5-tert-butyl-2-[1,4]diazepan-1-yl-benzooxazole. ES MS m/z=274 (M+H).

### Step 3: Synthesis of [4-(5-tert-Butyl-benzooxazol-2-yl)-[1,4]diazepan-1-yl]-morpholin-4-ylmethanone

To a solution of 100 mg (0.37 mmol) of 5-tert-butyl-2-[1,4]diazepan-1-yl-benzooxazole in dichloromethane (1 ml) are added 57 mg (0.44 mmol) of N,N-diisopropylethylamine and 55 mg (0.37 mmol) of morpholine-4-carbonyl chloride. The reaction mixture is stirred for 16 h and concentrated under reduced pressure. The residue is dissolved in dichloromethane and washed with water. The organic layer is dried (Na₂SO₄), filtered and the filtrate is concentrated under reduced pressure. The crude product is purified by column chromatography (silica, eluent: ethyl acetate) to afford 138 mg of [4-(5-tert-butyl-benzooxazol-2-yl)-[1,4]diazepan-1-yl]-morpholin-4-yl-methanone. ES MS m/z=387 (M+H).

Examples in Table 12 Method 9 are prepared according to a similar procedure.

### Method 10: Synthesis of [4-(5-tert-Butyl-benzooxazol-2-yl)-[1,4]diazepan-1-yl]-(1,1-dioxo-1λ⁶-thiomorpholin-4-yl)-methanone

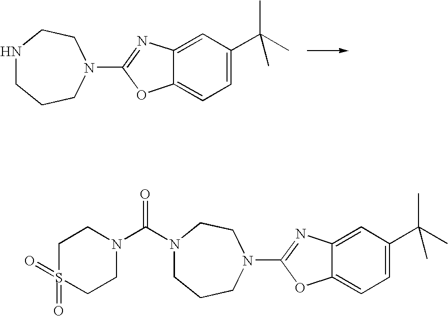

To a solution of 100 mg (0.37 mmol) of 5-tert-butyl-2-[1,4]diazepan-1-yl-benzooxazole (prepared according to step 1-2, Method 9) in dichloromethane (1 ml) are added 57 mg (0.44 mmol) of N,N-diisopropylethylamine and 72 mg (0.37 mmol) of 1,1-dioxo-1λ⁶-thiomorpholine-4-carbonyl chloride. The reaction mixture is stirred for 16 h at room temperature and then is concentrated under reduced pressure. The residue is dissolved in dichloromethane (8 mL) and washed with water (5 mL). The organic layer is dried (Na₂SO₄), filtered and the filtrate is concentrated under reduced pressure. The crude product is purified by column chromatography (silica, eluent: ethyl acetate) to afford 138 mg of [4-(5-tert-butyl-benzooxazol-2-yl)-[1,4]diazepan-1-yl]-(1,1-dioxo-1λ⁶-thiomorpholin-4-yl)-methanone. ES MS m/z=435 (M+H).

Examples in Table 10 Method 1 are prepared according to a similar procedure.

### Method 11: Synthesis of 5-tert-Butyl-2-[4-(3-chloro-5-trifluoromethyl-pyridin-2-yl)-[1,4]diazepan-1-yl]-benzooxazole

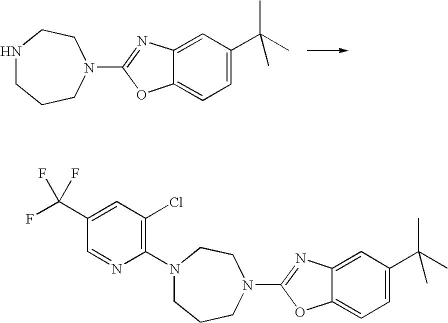

To a solution of 100 mg (0.35 mmol) of 5-tert-butyl-2-[1,4]diazepan-1-yl-benzooxazole (prepared according to step 1-2, Method 9) in ethoxyethanol (2 ml) are added 76 mg (0.35 mmol) of 2,3-dichloro-5-trifluoromethylpyridine and 136 mg (1.05 mmol) of N,N-diisopropylethylamine. The reaction mixture is heated in a sealed tube to 100° C. for 16 h. The reaction mixture is diluted with dichloromethane (8 mL) and washed with brine (5 mL). The organic layer is dried over Na₂SO₄, filtered and the filtrate is concentrated under reduced pressure. The crude product was purified by column chromatography (silica, eluent: dichloromethane) to afford 97 mg of 5-tert-butyl-2-[4-(3-chloro-5-trifluoromethyl-pyridin-2-yl)-[1,4]diazepan-1-yl]-benzooxazole. ES MS m/z=453, 455 (M+H).

Examples in Table 12 Method 11 are prepared according to a similar procedure.

### Method 12: Synthesis of Morpholin-4-yl-[4-(5-trifluoromethyl-benzooxazol-2-yl)-[1,4]diazepan-1-yl]-methanone

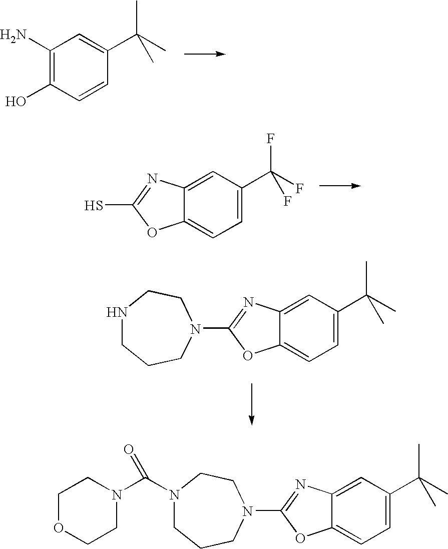

### Step 1: Synthesis of 4-(Morpholine-4-carbonyl)-[1,4]diazepane-1-carboxylic acid tert-butyl ester

To a solution of 500 mg (2.5 mmol) of [1,4]diazepane-1-carboxylic acid tert-butyl ester in anhydrous dichloromethane (5 mL) at room temperature are added 435 μL (2.5 mmol) of N,N-diisopropylethylamine and 287 μL (2.5 mmol) of morpholine-4-carbonyl chloride. The reaction mixture are stirred at room temperature for 1 h and then washed with saturated aqueous sodium bicarbonate solution (2 mL). The organic layer is dried (Na₂SO₄), filtered and the filtrate is concentrated under reduced pressure to afford 788 mg of 4-(morpholine-4-carbonyl)-[1,4]diazepane-1-carboxylic acid tert-butyl ester. ES MS m/z=314 (M+H).

### Step 2: Synthesis of [1,4]Diazepan-1-yl-morpholin-4-yl-methanone

To a solution of 788 mg (2.5 mmol) of 4-(morpholine-4-carbonyl)-[1,4]diazepane-1-carboxylic acid tert-butyl ester in methanol (4 mL) are added dropwise 12N HCl (4 mL). The reaction mixture are stirred at room temperature for 1.5 h and then concentrated under reduced pressure to constant weight to afford 872 mg of [1,4]diazepan-1-yl-morpholin-4-yl-methanone as its hydrochloride salt. ES MS m/z=214 (M+H).

### Step 3: Synthesis of Morpholin-4-yl-[4-(5-trifluoromethyl-benzooxazol-2-yl)-[1,4]diazepan-1-yl]-methanone

To a solution of 114 mg (0.46 mmol) of [1,4]diazepan-1-yl-morpholin-4-yl-methanone in toluene (2 mL) is added 239 μL (1.37 mmol) of N,N-diisopropylethylamine and 100 mg (0.46 mmol) of 5-trifluoromethyl-benzooxazole-2-thiol (prepared according to step 1-2, Method 5). The reaction mixture is stirred at 100° C. for 16 h. The mixture is washed with ammonium hydroxide (2 mL) and filtered. The filtrate is extracted with dichloromethane (3×2 mL). The combined organic extracts are dried (Na₂SO₄), filtered and the filtrate is concentrated under reduced pressure. The crude mixture is purified by column chromatography (silica, eluent: dichloromethane, 0-1% methanol) to afford 57 mg of morpholin-4-yl-[4-(5-trifluoromethyl-benzooxazol-2-yl)-[1,4]diazepan-1-yl]-methanone. ES MS m/z=399 (M+H).

Examples in Table 12 Method 12 are prepared according to a similar procedure.

### Method 13: Synthesis of 4-[3-chloro-5-(trifluoromethyl)pyridin-2-yl]-N-(4-methyl-1,3-thiazol-2-yl)-1,4-diazepane-1-carboxamide

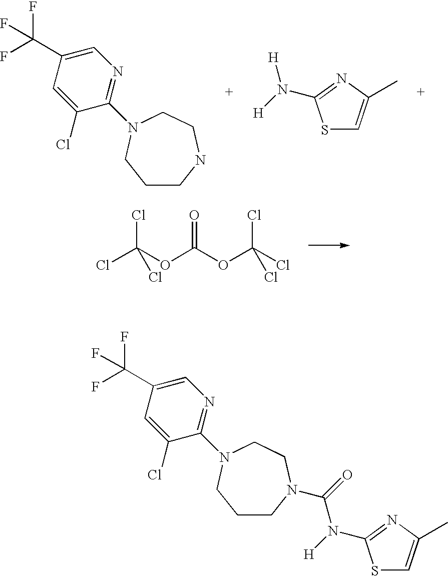

To a solution of 42 mg (0.143 mmol) of triphosgene in 2 mL of dichloromethane is added a solution of 41 mg (0.358 mmol) 2-amino-5-methylthiazole and 0.069 μL N,N-diisopropylethylamine in 3.6 mL dichloromethane dropwise. Upon completion of addition, the solution is stirred a further 5 minutes before adding a solution of 100 mg (0.358 mmol) diazepane and 0.069 μL N,N-diisopropylethylamine in 2 mL dichloromethane. The reaction is stirred for 30 minutes at room temperature before washing with aqueous saturated sodium bicarbonate solution and brine. The organic layer is dried (Na₂SO₄), filtered and concentrated in vacuo. Purification was accomplished by reverse phase HPLC to afford 35.7 mg of 4-[3-chloro-5-(trifluoromethyl)pyridin-2-yl]-N-(4-methyl-1,3-thiazol-2-yl)-1,4-diazepane-1-carboxamide. ES MS m/z 420 (M+H)

Examples in Table 12 Method 13 are prepared according to a similar procedure.

### Method 14: Synthesis of 4-(3,5-Dichloro-pyridin-2-yl)-[1,4]diazepane-1-carboxylic acid (5-tert-butyl-isoxazol-3-yl)-amide

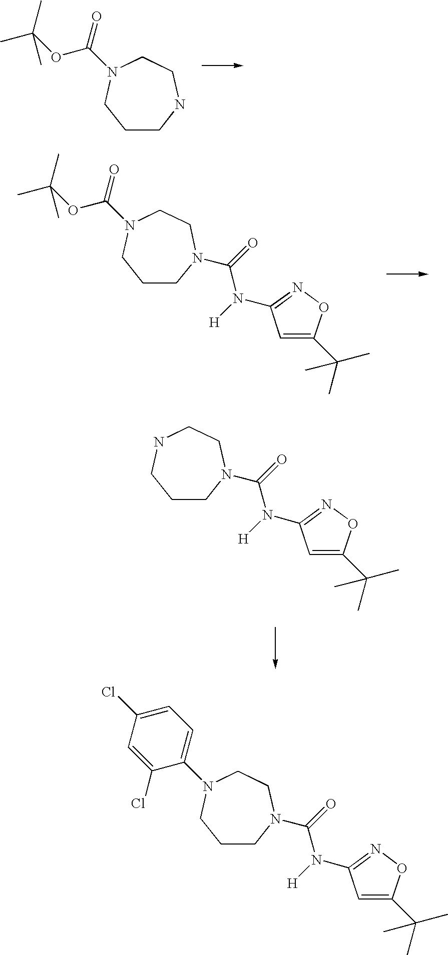

### Step 1: Synthesis of 4-(5-tert-Butyl-isoxazol-3-ylcarbamoyl)-[1,4]diazepane-1-carboxylic acid tent-butyl ester

To a solution of 2.936 g (9.986 mmol) triphosgene in 150 mL dichloromethane is added a solution consisting of 3.5 g (24.965 mmol) 5-tert-butyl-isoxazol-3-ylamine and 4.7 mL (27.461 mmol) N,N-diisopropylethylamine in 250 mL dichloromethane dropwise. Upon completion of addition, the solution is stirred for a further 5 minutes before adding a solution of 5 g (24.965 mmol) [1,4]diazepane-1-carboxylic acid tert-butyl ester and 4.7 mL (27.461 mmol) diisopropylethylamine in 140 mL dichloromethane. The reaction is stirred for 30 minutes at room temperature and then washed with aqueous saturated sodium bicarbonate solution and brine. The organics are dried (Na₂SO₄), filtered and concentrated in vacuo to afford 9.696 g of 4-(5-tert-butyl-isoxazol-3-ylcarbamoyl)-[1,4]diazepane-1-carboxylic acid tert-butyl ester. ES MS m/z 367 (M+H).

### Step 2: Synthesis of [1,4]Diazepane-1-carboxylic acid (5-tent-butyl-isoxazol-3-yl)-amide

To a solution of 2 g (5.458 mmol) 4-(5-tert-butyl-isoxazol-3-ylcarbamoyl)-[1,4]diazepane-1-carboxylic acid tert-butyl ester in 22 mL dichloromethane is added 6.823 mL (27.290 mmol) 4 N HCl in dioxanes. The reaction is stirred at room temperature overnight and concentrated. The oil is diluted with dichloromethane and washed with aqueous saturated sodium bicarbonate solution and brine. The solution is dried (Na₂SO₄), filtered and concentrated in vacuo to afford [1,4]diazepane-1-carboxylic acid (5-tert-butyl-isoxazol-3-yl)-amide. ES MS m/z 267 (M+H).

According to this method the following [1,4]diazepane-1-carboxylic acid amide is synthesized.

TABLE 8

| | | Yield | m/z |
|---|---|---|---|
| Structure | Name | (%) | [M + H]+ |
| 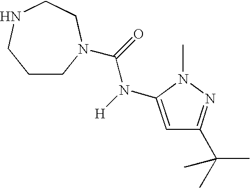 | [1,4]Diazepane-1-carboxylic acid (5-tert-butyl-2-methyl-2H-pyrazol- 3-yl)-amide | 100 | 280 |

### Step 3: Synthesis of 4-(3,5-Dichloro-pyridin-2-yl)-[1,4]diazepane-1-carboxylic acid (5-tert-butyl-isoxazol-3-yl)-amide

To a solution of 0.1 g (0.375 mmol) [1,4]diazepane-1-carboxylic acid (5-tert-butyl-isoxazol-3-yl)-amide in 1.5 mL dimethylsulfoxide is added 0.042 g (0.25 mmol) 3,5-dichloro-2-fluoropyridine and 0.75 mL (0.75 mmol) 1M sodium hexadimethyldisilazide. The mixture is heated at 60 C overnight in a sealed tube. The mixture is cooled to room temperature and filtered through a fritted cartridge. Purification is done by reverse phase HPLC to afford 11.9 mg of 4-(3,5-dichloro-pyridin-2-yl)-[1,4]diazepane-1-carboxylic acid (5-tert-butyl-isoxazol-3-yl)-amide. ES MS m/z 412 (M+H).

Examples in Table 12 Method 14 are prepared according to a similar procedure.

### Method 15: Synthesis of 6-Chloro-2-[4-(3-chloro-5-trifluoromethyl-pyridin-2-yl)-[1,4]diazepan-1-yl]-benzothiazole

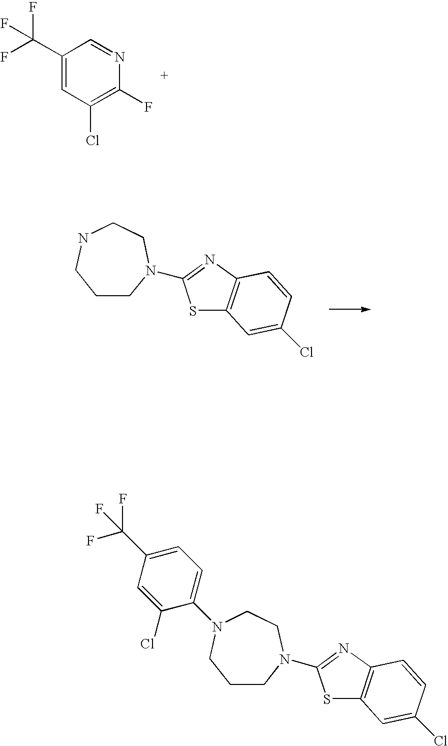

This compound is prepared according to a similar procedure as Method 14 Step 3.

Examples in Table 12 Method 15 are prepared according to a similar procedure.

### Method 16: Synthesis of [4-(6-Chloro-benzothiazol-2-yl)-[1,4]diazepan-1-yl]-(tetrahydro-pyran-4-yl)-methanone

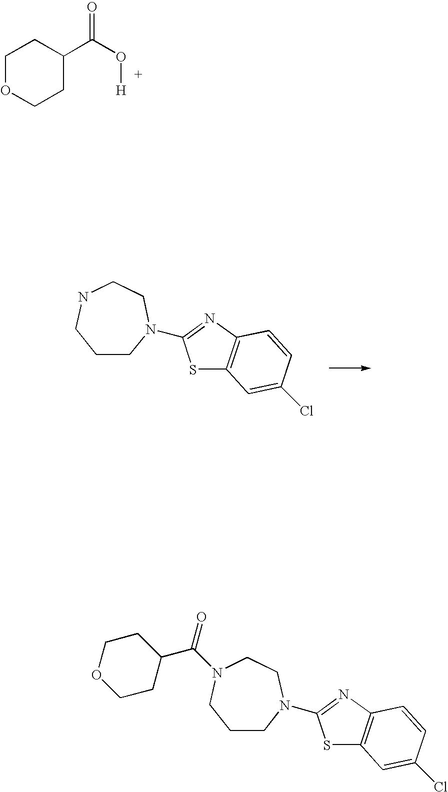

To a solution of 0.1 g (0.373 mmol) 6-chloro-2-[1,4]diazepan-1-yl-benzothiazole in 3 mL dimethylformamide is added 0.058 g (0.448 mmol) of tetrahydro-pyran-4-carboxylic acid tert-butyl ester, 0.086 g (0.448 mmol) of N-(3-dimethylaminopropyl)-N′-ethylcarbodiimide, 0.060 g (0.448 mmol) 1-hydroxybenzotriazole, and 0.065 mL (0.373 mmol) N,N-diisopropylethylamine. The mixture is stirred overnight at room temperature and then diluted with dichloromethane and washed with 2×3 mL aqueous saturated sodium bicarbonate solution, 2×3 mL 1N HCl solution, and 1×3 mL brine solution. The organics are dried (Na₂SO₄), filtered and concentrated. The residue is taken up in a small amount of dimethylformamide and purified by reverse phase HPLC to afford 0.0764 g of [4-(6-chloro-benzothiazol-2-yl)-[1,4]diazepan-1-yl]-(tetrahydro-pyran-4-yl)-methanone. ES MS m/z 380 (M+H).

Examples in Table 12 Method 16 are prepared according to a similar procedure.

### Method 17: Synthesis of [4-(1H-Benzoimidazol-2-yl)-[1,4]diazepan-1-yl]-(1,1-dioxo-1λ⁶-thiomorpholin-4-yl)-methanone

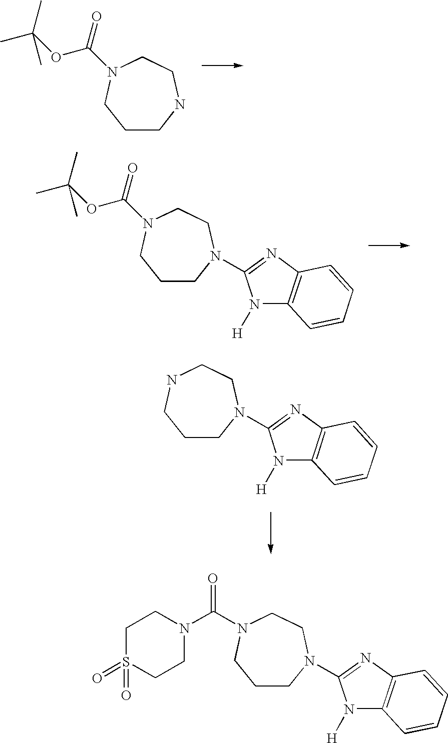

### Step 1: Synthesis of 4-(1H-Benzoimidazol-2-yl)-[1,4]diazepane-1-carboxylic acid tert-butyl ester

Prepared as described by adaptation of the following reference: J. Med. Chem., 2006, 49, 3719

A microwave reaction vial is charged with 0.973 mL (5 mmol) [1,4]diazepane-1-carboxylic acid tert-butyl ester, 0.763 g (5 mmol) 2-chlorobenzimidazole, 1.394 mL (10 mmol) triethylamine, and 4 mL ethanol. The vial is heated at 175 C for 1 hour. The mixture is filtered through a cartridge and purified by flash chromatography to afford 1.1 g of 4-(1H-benzoimidazol-2-yl)-[1,4]diazepane-1-carboxylic acid tert-butyl ester. ES MS m/z 317 (M+H).

According to this method the following intermediates are synthesized.

TABLE 9

| | | Yield | m/z |
|---|---|---|---|
| Structure | Name | (%) | [M + H]+ |
| 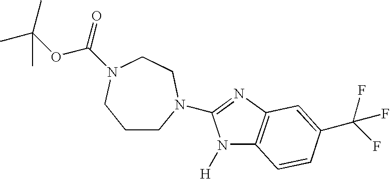 | 4-(5-trifluoromethyl-5- trifluoromethyl-1H- benzoimidazole)-[1,4]diazepane- 1-carboxylic acid tert-butyl ester | 59 | 389 |

### Step 2: Synthesis of 2-[1,4]Diazepan-1-yl-1H-benzoimidazole

8.668 mL (34.67 mmol) of 4N HCl in dioxanes is added to 4-(1H-benzoimidazol-2-yl)-[1,4]diazepane-1-carboxylic acid tert-butyl ester and the mixture stirred overnight at room temperature. An additional 4 mL of 4N HCl in dioxanes is added to the reaction mixture and the reaction as well as a small amount of methanol to make the solution heterogeneous. The mixture is stirred overnight at room temperature and then concentrated in vacuo and used crude in subsequent reactions.

According to this method the following intermediates are synthesized.

TABLE 10

| | | Yield | m/z |
|---|---|---|---|
| Structure | Name | (%) | [M + H]+ |
| 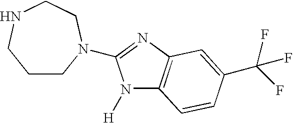 | 2-[1,4]Diazepan-1-yl-5- trifluoromethyl-1H-benzoimidazole | 100 | 285 |

### Step 3: Synthesis of 1,1-Dioxo-1λ⁶-thiomorpholine-4-carbonyl chloride

1 g (7.397 mmol) of 1,1-Dioxo-1λ⁶-thiomorpholine is dispersed in 50 mL tetrahydrofuran followed by the addition of 1.238 mL (8.80 mmol) triethylamine and 11.743 mL (22.2 mmol) 20% phosgene in toluene. The reaction mixture is stirred at room temperature overnight and then diluted with ether and filtered through Celite®. The Celite® is washed with ether and the filtrate is concentrated and dried under high vacuum to obtain 1.425 g of off white solid which is 1,1-dioxo-1λ⁶-thiomorpholine-4-carbonyl chloride.

### Step 4: Synthesis of [4-(1H-Benzoimidazol-2-yl)-[1,4]diazepan-1-yl]-(1,1-dioxo-1λ⁶-thiomorpholin-4-yl)-methanone

To a solution of 0.15 g (0.593 mmol) 2-[1,4]diazepan-1-yl-1H-benzoimidazole in 3.5 mL tetrahydrofuran is added 0.248 mL (1.423 mmol) N,N-diisopropylethylamine and 0.117 g (0.593 mmol) 1,1-dioxo-1λ⁶-thiomorpholine-4-carbonyl chloride. The mixture is stirred at room temperature overnight then filtered and purified by reverse phase HPLC to afford 0.108 g of [4-(1H-benzoimidazol-2-yl)-[1,4]diazepan-1-yl]-(1,1-dioxo-1λ⁶-thiomorpholin-4-yl)-methanone. ES MS m/z 378 (M+H).

Examples in Table 12 Method 17 are prepared according to a similar procedure.

### Method 18: Synthesis of Morpholin-4-yl-[4-(6-trifluoromethyl-1H-benzoimidazol-2-yl)-[1,4]diazepan-1-yl]-methanone

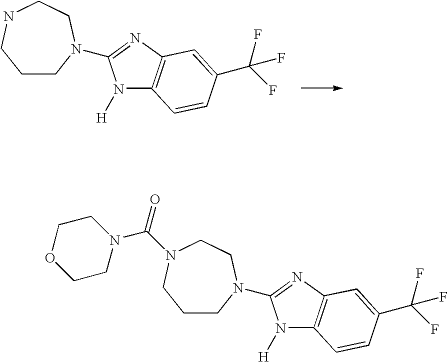

To a solution of 0.190 g (0.593 mmol) 2-[1,4]diazepan-1-yl-5-trifluoromethyl-1H-benzoimidazole hydrochloride (made as described in Method 17 Steps 1-2) in 3.5 mL tetrahydrofuran is added 0.069 mL (0.593 mmol) morpholine-4-carbonyl chloride and 0.248 mL (1.423 mmol) N,N-diisopropylethylamine. The reaction mixture is stirred overnight at room temperature and then filtered and purified by reverse phase HPLC to afford 0.120 g morpholin-4-yl-[4-(6-trifluoromethyl-1H-benzoimidazol-2-yl)-[1,4]diazepan-1-yl]-methanone. ES MS m/z 398 (M+H).

Examples in Table 12 Method 18 are prepared according to a similar procedure.

### Method 19: Synthesis of [4-(5-Chloro-benzooxazol-2-yl)-[1,4]diazepan-1-yl]-(tetrahydro-pyran-4-yl)-methanone

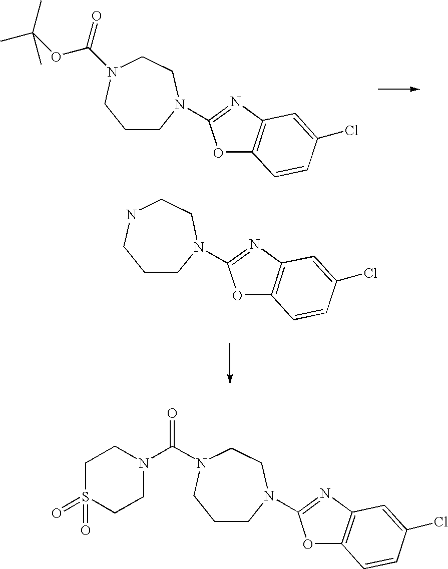

### Step 1: Synthesis of 5-Chloro-2-[1,4]diazepan-1-yl-benzooxazole

To 0.2 g (0.568 mmol) 4-(5-chloro-benzooxazol-2-yl)-[1,4]diazepane-1-carboxylic acid tert-butyl ester (synthesis described in Method 5: steps 2-3) is added 5 mL (20 mmol) 4 N HCl in dioxanes and the reaction is stirred at room temperature overnight. The mixture is concentrated in vacuo and diluted with dichloromethane and washed with aqueous saturated sodium bicarbonate solution to free base the diazepane. The organic layer is dried (Na₂SO₄) and concentrated in vacuo to afford quantitatively 5-chloro-2-[1,4]diazepan-1-yl-benzooxazole. ES MS m/z 252 (M+H).

According to this method the following [1,4]dizaepan-1-yl-benzooxazoles were synthesized.

TABLE 11

| | | Yield | m/z |
|---|---|---|---|
| Structure | Name | (%) | [M + H]+ |
| 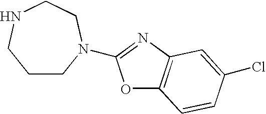 | 5-Chloro-2-[1,4]diazepan-1-yl- benzooxazole | 72 | 252.2 and 254.2 |
| 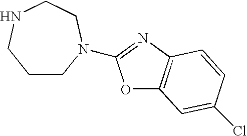 | 6-Chloro-2-[1,4]diazepan-1-yl- benzooxazole | 52 | 252.2 and 254.2 |
| 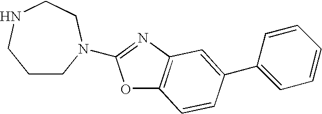 | 2-[1,4]Diazepan-1-yl-5-phenyl- benzooxazole | 63 | 294.4 |
| 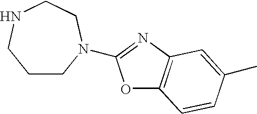 | 2-[1,4]Diazepan-1-yl-5-methyl- benzooxazole | 71 | 232.2 |
| 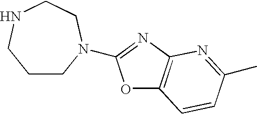 | 2-[1,4]Diazepan-1-yl-5-methyl- oxazolo[4,5-b]pyridine | 62 | 233 |

### Step 2: Synthesis of [4-(5-Chloro-benzooxazol-2-yl)-[1,4]diazepan-1-yl]-(tetrahydro-pyran-4-yl)-methanone

To a solution of 0.113 g (0.449 mmol) 5-chloro-2-[1,4]diazepan-1-yl-benzooxazole in 2 mL of dimethylformamide is added 0.070 g (0.539 mmol) of tetrahydro-pyran-4-carboxylic acid tert-butyl ester, 0.103 g (0.539 mmol) of N-(3-dimethylaminopropyl)-N′-ethylcarbodiimide, 0.073 g (0.539 mmol) 1-hydroxybenzotriazole, and 0.005 g (0.045 mmol) dimethylaminopyridine. The mixture is stirred at room temperature overnight, filtered through a fritted cartridge and purified by reverse phase HPLC to afford 0.061 g of [4-(5-chloro-benzooxazol-2-yl)-[1,4]diazepan-1-yl]-(tetrahydro-pyran-4-yl)-methanone. ES MS m/z 364 (M+H).

Examples in Table 12 Method 19 are prepared according to a similar procedure.

### Method 20: Synthesis of [4-(5-Chloro-benzooxazol-2-yl)-[1,4]diazepan-1-yl]-morpholin-4-ylmethanone

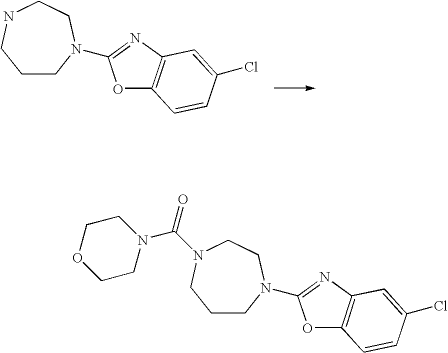

To a solution of 0.113 g (0.449 mmol) 5-chloro-2-[1,4]diazepan-1-yl-benzooxazole in 2 mL of dimethylformamide is added 0.052 mL (0.449 mmol) morpholine carbonylchloride and 0.094 mL (0.539 mmol) N,N-diisopropylethylamine. The mixture is stirred overnight at room temperature, filtered and purified by reverse phase HPLC to afford 0.077 g [4-(5-chloro-benzooxazol-2-yl)-[1,4]diazepan-1-yl]-morpholin-4-yl-methanone. ES MS m/z 365 (M+H).

Examples in Table 12 Method 20 are prepared according to a similar procedure.

### Method 21: Synthesis of (4-Trifluoromethyl-phenyl)-[4-(5-trifluoromethyl-pyridin-2-yl)-[1,4]diazepan-1-yl]-methanone

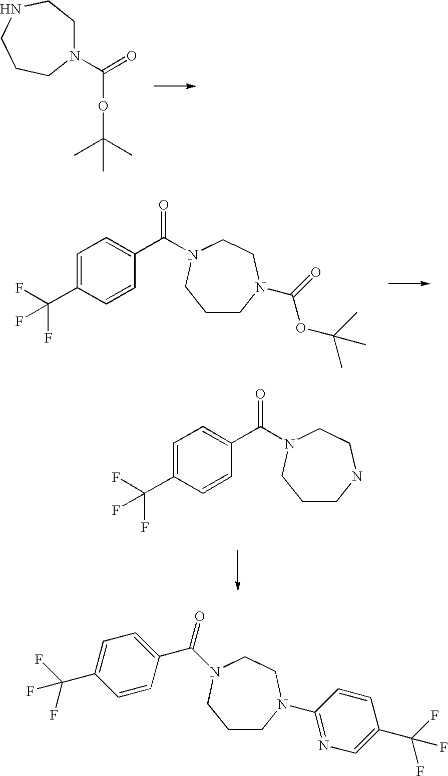

### Step 1: Synthesis of 4-(4-Trifluoromethyl-benzoyl)-[1,4]diazepane-1-carboxylic acid tert-butyl ester

To a solution of 1.00 g (5.26 mmol) p-trifluoromethylbenzoic acid and 1.15 g (8.50 mmol) 1-hydroxybenzotriazole in 50 mL anhydrous dimethylformamide is added 1.63 g (8.50 mmol) N,(3-dimethylaminopropyl)-N′-ethylcarbodiimide. After stirring 20 min 0.80 mL (4.10 mmol) of the [1,4]diazepane-1-carboxylic acid tert-butyl ester is added, followed by a catalytic amount of dimethylaminopyridine, and the mixture is stirred over a number of days. Water and ethyl acetate are added and the layers are separated. The organic layer is washed with water, then saturated aqueous bicarbonate solution, then brine. The organic layer is then dried (Na₂SO₄), filtered, and the solvent removed in vacuo to afford a yellow oil, 1.5 g. The product 4-(4-trifluoromethyl-benzoyl)-[1,4]diazepane-1-carboxylic acid tert-butyl ester is purified by flash column chromatography on SiO₂ using 0-50% EtOAc in hexanes first, followed by 5-10% MeOH in dichloromethane mixtures as eluent. The product-rich fractions are collected and the solvent is removed in vacuo to afford 1.42 g of product as a pale yellow oil, ES MS (+) m/z 273 (M-BoCH+).

### Step 2: Synthesis of [1,4]Diazepan-1-yl-(4-trifluoromethyl-phenyl)-methanone hydrochloride salt

1.4 g (3.76 mmol) of 4-(4-trifluoromethyl-benzoyl)-[1,4]diazepane-1-carboxylic acid tert-butyl ester is dissolved in 50 mL dichloromethane and treated at room temperature with 15 mL (15 mmol) of 1M HCl solution in 1,4-dioxane. The mixture is left stirring for 5 h, then the solvent is removed in vacuo to afford 1.2 g of the product as a white solid, which is used as is without purification. ES MS m/z 273 (M+H).

### Step 3: Synthesis of (4-Trifluoromethyl-phenyl)-[4-(5-trifluoromethyl-pyridin-2-yl)-[1,4]diazepan-1-yl]-methanone

To 0.110 g (0.39 mmol.) of [1,4]diazepan-1-yl-(4-trifluoromethyl-phenyl)-methanone hydrochloride salt in 1.5 mL anhydrous dimethylsulfoxide is added 0.2 mL (1.15 mmol) N,N-diisopropylethylamine, followed by 0.1 g (0.55 mmol) of 5-trifluoromethyl-2-chloropyridine and a catalytic amount of dimethylaminopyridine. The mixture is left stirring at room temperature over 2 days, before water and ethyl acetate are added. The layers are separated and the organic phase is washed sequentially with water, saturated aqueous sodium bicarbonate solution, then brine. The organic layer is then dried (Na₂SO₄), filtered, and the solvent is removed in vacuo. LC-MS of crude shows both product (12.7% area) and starting diazapane amide (44.7% area) peaks. The product is purified by flash column chromatography on SiO₂ using 0-5% methanol in dichloromethane mixtures as eluent. The desired product is isolated as an oil (40 mg). Further purification by preparative-HPLC-MS affords 25 mg of a colorless foam. ES MS m/z 418 (M+H).

Examples in Table 12 Method 21 are prepared according to a similar procedure.

### Method 22: Synthesis of 4-(5-Trifluoromethyl-pyridin-2-yl)-[1,4]diazepane-1-carboxylic acid (4-trifluoromethyl-phenyl)-amide

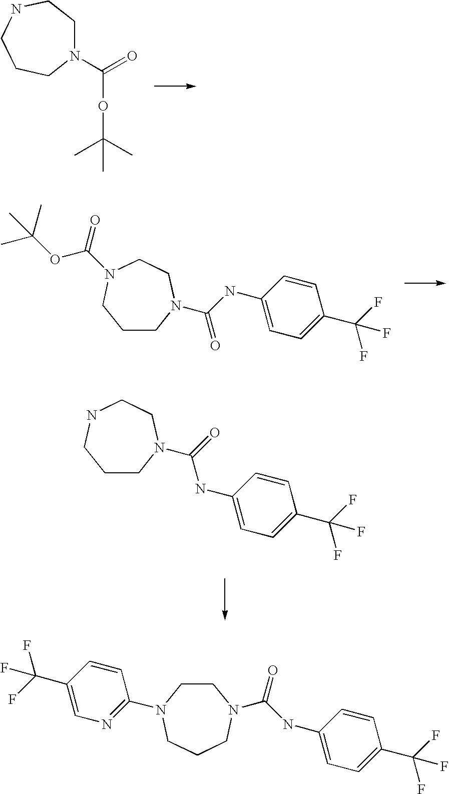

### Step 1: Synthesis of 4-(4-Trifluoromethyl-phenylcarbamoyl)-[1,4]diazepane-1-carboxylic acid tert-butyl ester

To a solution of 0.78 mL (3.98 mmol) [1,4]diazepane-1-carboxylic acid tert-butyl ester in 20 mL anhydrous tetrahydrofuran is added 0.56 mL (4 mmol) p-trifluoromethyl-phenyl isocyanate and the mixture is left stiffing overnight. The solvent is then removed in vacuo to afford a white foam, which recrystallizes from hot ethyl acetate to yield a white solid, 0.71 g (contains 2.3% of symmetrical bis-phenyl-urea). ES MS m/z 388 (M+H).

### Step 2: Synthesis of [1,4]Diazepane-1-carboxylic acid (4-trifluoromethyl-phenyl)-amide hydrochloride salt

0.71 g (1.83 mmol) of 4-(4-trifluoromethyl-phenylcarbamoyl)-[1,4]diazepane-1-carboxylic acid tert-butyl ester is dissolved in 30 mL anhydrous dichloromethane and is treated at room temperature with 2.5 mL (10 mmol) 4 M HCl in dioxane solution. The mixture is left stiffing vigorously overnight and then the solvent is removed in vacuo and the product isolated in quantitative yield is used as is without purification ES MS m/z 288 (M+H).

### Step 3: Synthesis of 4-(5-Trifluoromethyl-pyridin-2-yl)-[1,4]diazepane-1-carboxylic acid (4-trifluoromethyl-phenyl)-amide

This compound is made according to Method 22 Step 3 to afford 0.014 g of 445-trifluoromethyl-pyridin-2-yl)-[1,4]diazepane-1-carboxylic acid (4-trifluoromethyl-phenyl)-amide. ES MS m/z 433 (M+H).

Examples in Table 12 Method 22 are prepared according to a similar procedure.

### Method 23: Synthesis of 4-(5-Ethyl-pyrimidin-2-yl)-[1,4]diazepane-1-carboxylic acid (5-tert-butyl-isoxazol-3-yl)-amide

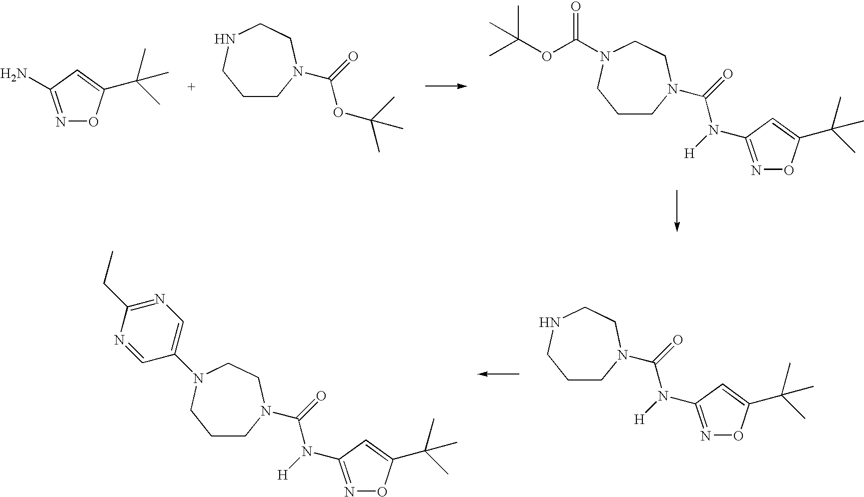

### Step 1: Synthesis of 4-(5-tert-Butyl-isoxazol-3-ylcarbamoyl)-[1,4]diazepane-1-carboxylic acid tent-butyl ester

Triphosgene (3.05 g; 10.3 mmol) is dissolved in 120 mL dichloromethane. A mixture of 3-amino-5-tert-butyl-isoxazole (3.60 g; 25.7 mmol) and N,N-diisopropylethylamine (4.9 mL; 28.2 mmol) in 210 mL dichloromethane is slowly added dropwise over 2 hours via an addition funnel. After the reaction is stirred for an additional 30 minutes, a solution of the Boc-protected diazapane (5 mL; 25.7 mmol) and 4.9 mL N,N-diisopropylethyl amine in 40 mL dichloromethane is added rapidly in one portion. The mixture is stirred for 3 hours at room temperature, then washed with saturated aqueous sodium bicarbonate solution. The organic fraction is dried (Na₂SO₄), filtered, and the solvent is removed in vacuo to afford 10.3 g of crude material. The solid is recrystallized from hot ethyl acetate to afford 5.45 g of product as a pure, white solid. ES MS m/z 367 (M+H).

### Step 2: Synthesis of [1,4]Diazepane-1-carboxylic acid (5-tent-butyl-isoxazol-3-yl)-amide

4-(5-tert-Butyl-isoxazol-3-ylcarbamoyl)-[1,4]diazepane-1-carboxylic acid tert-butyl ester (330 mg; 0.90 mmol. 1 equiv.) is dissolved in 5 mL dichloromethane and treated at room temperature with HCl in dioxane (4N, 1.13 mL; 4.5 mmol; 5 equiv.). The mixture is stirred for 3 hours, during which time a white precipitate formed. Evaporation of solvent in vacuo afforded an off-white foam, 272 mg. The crude product is used without purification. ES MS m/z 267 (M+H).

### Step 3: Synthesis of 4-(5-Ethyl-pyrimidin-2-yl)-[1,4]diazepane-1-carboxylic acid (5-tert-butyl-isoxazol-3-yl)-amide

[1,4]-Diazepane-1-carboxylic acid (5-tert-butyl-isoxazol-3-yl)-amide (45 mg; 0.15 mmol; 1 equiv.) dissolved in 1 mL of de-gassed 1-methyl-2-pyrrolidone is added to 2-chloro-5-ethylpyrimidine (43 mg; 0.30 mmol; 2 equiv.), potassium carbonate (104 mg; 0.75 mmol; 5 equiv.), and copper powder (9.5 mg, 0.15 mmol, 1 equiv.). The reaction is placed on an orbital shaker heated at 80° C. for 16 hours. The reaction is filtered and purified by mass triggered reverse phase HPLC then silica gel prep plate eluted with 50% ethyl acetate/hexanes to afford pure product, 2.7 mg.

Examples in Table 12 Method 23 are prepared according to a similar procedure.

### Method 24: Synthesis of (4-Furo[3,2-c]pyridin-4-yl-[1,4]diazepan-1-yl)-morpholin-4-yl-methanone

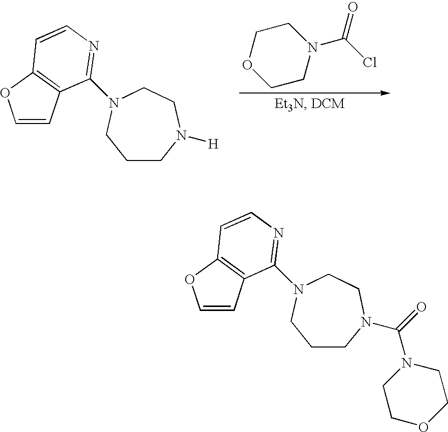

To a solution of 4-[1,4]diazepan-1-yl-furo[2,3-c]pyridine (33 mg, 0.15 mmol; 1 equiv.) in 1.5 mL of dichloromethane is added triethylamine (45 mg; 0.45 mmol; 3 equiv.) and 4-morpholinecarbonyl chloride (34 mg; 0.225 mmol, 1.5 equiv.) and placed on an orbital shaker for 16 hours. The solvent is removed in vacuo to afford a yellow oil. Purification by mass triggered reverse phase HPLC afforded the pure product, 30 mg.

Examples in Table 12 Method 24 are prepared according to a similar procedure.

TABLE 12
Examples

| Example | Structure | Name | m/z | method |
|---|---|---|---|---|
| 1 | 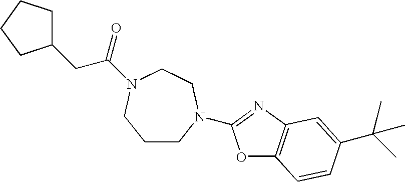 | 1-[4-(5-tert-Butyl- benzooxazol-2-yl)- [1,4]diazepan-1-yl]-2- cyclopentyl-ethanone | 384 | 1 |
| 2 | 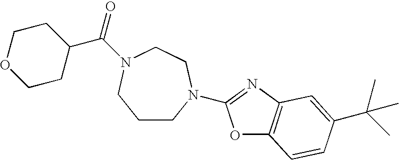 | [4-(5-tert-Butyl- benzooxazol-2-yl)- [1,4]diazepan-1-yl]- (tetrahydro-pyran-4-yl)- methanone | 386 | 1 |
| 3 | 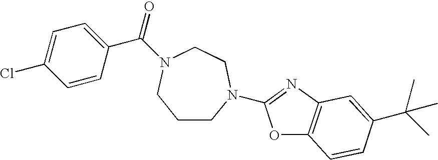 | [4-(5-tert-Butyl- benzooxazol-2-yl)- [1,4]diazepan-1-yl]-(4- chloro-phenyl)-methanone | 412/414 | 1 |
| 4 | 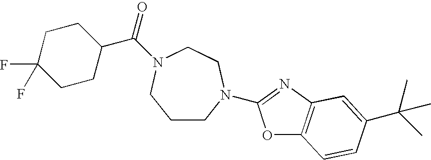 | [4-(5-tert-Butyl- benzooxazol-2-yl)- [1,4]diazepan-1-yl]-(4,4- difluoro-cyclohexyl)- methanone | 420 | 1 |
| 5 | 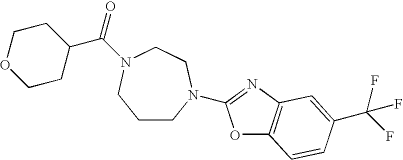 | (Tetrahydro-pyran-4-yl)-[4- (5-trifluoromethyl- benzooxazol-2-yl)- [1,4]diazepan-1-yl]- methanone | 398 | 1 |
| 6 | 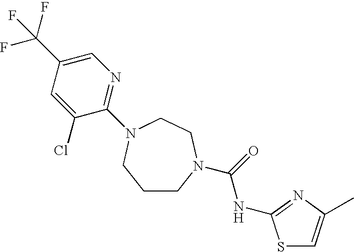 | 4-(3-Chloro-5- trifluoromethyl-pyridin-2- yl)-[1,4]diazepane-1- carboxylic acid (4-methyl- thiazol-2-yl)-amide | 420 | 13 |
| 7 | 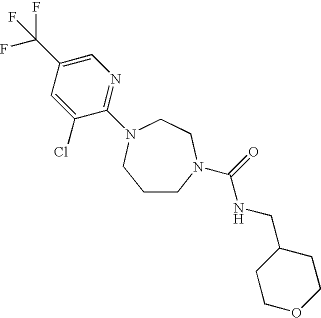 | 4-(3-Chloro-5- trifluoromethyl-pyridin-2- yl)-[1,4]diazepane-1- carboxylic acid (tetrahydro- pyran-4-ylmethyl)-amide | 421 | 13 |
| 8 | 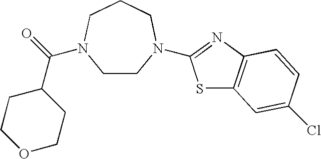 | [4-(6-Chloro-benzothiazol- 2-yl)-[1,4]diazepan-1-yl]- (tetrahydro-pyran-4-yl)- methanone | 380 | 16 |
| 9 | 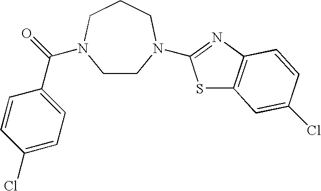 | [4-(6-Chloro-benzothiazol- 2-yl)-[1,4]diazepan-1-yl]- (4-chloro-phenyl)- methanone | 406 | 16 |
| 10 | 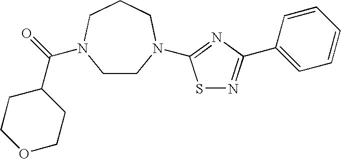 | [4-(3-Phenyl- [1,2,4]thiadiazol-5-yl)- [1,4]diazepan-1-yl]- (tetrahydro-pyran-4-yl)- methanone | 373 | 16 |
| 11 | 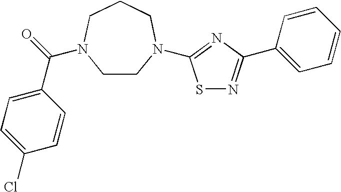 | (4-Chloro-phenyl)-[4-(3- phenyl-[1,2,4]thiadiazol-5- yl)-[1,4]diazepan-1-yl]- methanone | 399 | 16 |
| 12 | 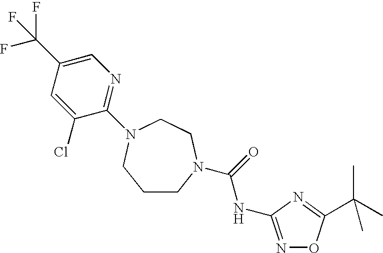 | 4-(3-Chloro-5- trifluoromethyl-pyridin-2- yl)-[1,4]diazepane-1- carboxylic acid (5-tert- butyl-isoxazol-3-yl)-amide | 446 | 13 |
| 13 | 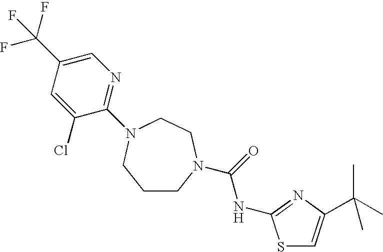 | 4-(3-Chloro-5- trifluoromethyl-pyridin-2- yl)-[1,4]diazepane-1- carboxylic acid (4-tert- butyl-thiazol-2-yl)-amide | 462 | 13 |
| 14 | 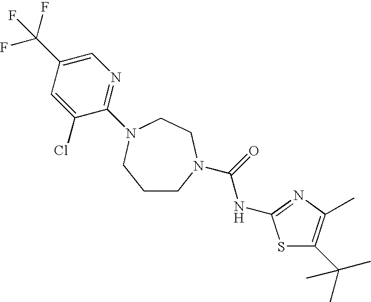 | 4-(3-Chloro-5- trifluoromethyl-pyridin-2- yl)-[1,4]diazepane-1- carboxylic acid (5-tert- butyl-4-methyl-thiazol-2- yl)-amide | 476 | 13 |
| 15 | 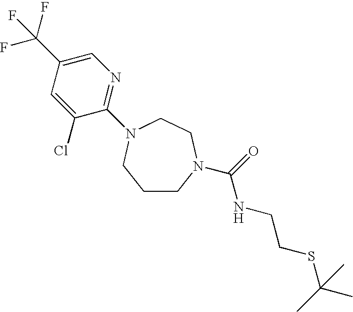 | 4-(3-Chloro-5- trifluoromethyl-pyridin-2- yl)-[1,4]diazepane-1- carboxylic acid (2-tert- butylsulfanyl-ethyl)-amide | 439 | 13 |
| 16 | 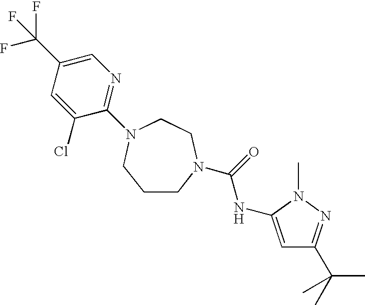 | 4-(3-Chloro-5- trifluoromethyl-pyridin-2- yl)-[1,4]diazepane-1- carboxylic acid (5-tert- butyl-2-methyl-2H- pyrazol-3-yl)-amide | 459 | 13 |
| 17 | 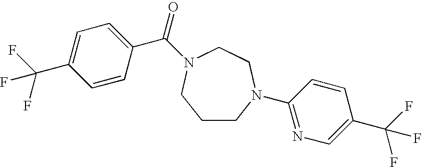 | (4-Trifluoromethyl- phenyl)-[4-(5- trifluoromethyl-pyridin-2- yl)-[1,4]diazepan-1-yl]- methanone | 418 | 21 |
| 18 | 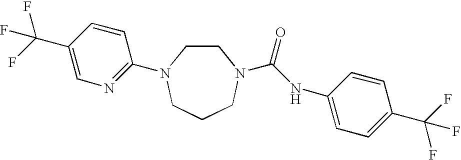 | 4-(5-Trifluoromethyl- pyridin-2-yl)- [1,4]diazepane-1- carboxylic acid (4- trifluoromethyl-phenyl)- amide | 433 | 22 |
| 19 | 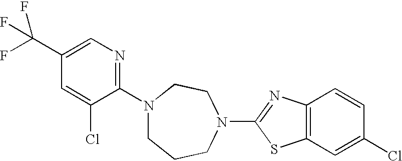 | 6-Chloro-2-[4-(3-chloro-5- trifluoromethyl-pyridin-2- yl)-[1,4]diazepan-1-yl]- benzothiazole | 448 | 15 |
| 20 | 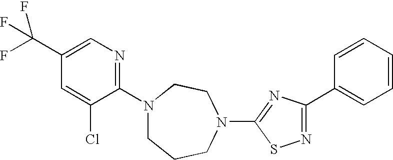 | 1-(3-Chloro-5- trifluoromethyl-pyridin-2- yl)-4-(3-phenyl- [1,2,4]thiadiazol-5-yl)- [1,4]diazepane | 448 | 15 |
| 21 | 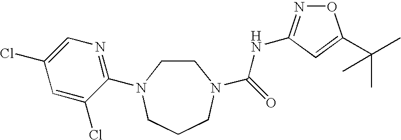 | 4-(3,5-Dichloro-pyridin-2- yl)-[1,4]diazepane-1- carboxylic acid (5-tert- butyl-isoxazol-3-yl)-amide | 412 | 14 |
| 22 | 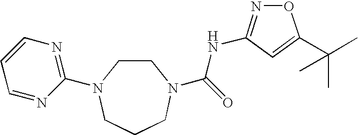 | 4-Pyrimidin-2-yl- [1,4]diazepane-1- carboxylic acid (5-tert- butyl-isoxazol-3-yl)-amide | 344 | 14 |
| 23 | 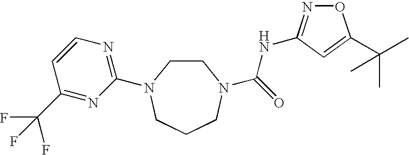 | 4-(4-Trifluoromethyl- pyrimidin-2-yl)- [1,4]diazepane-1- carboxylic acid (5-tert- butyl-isoxazol-3-yl)-amide | 413 | 14 |
| 24 | 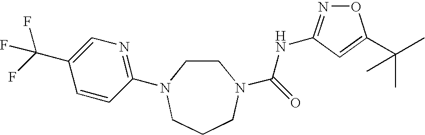 | 4-(5-Trifluoromethyl- pyridin-2-yl)- [1,4]diazepane-1- carboxylic acid (5-tert- butyl-isoxazol-3-yl)-amide | 412 | 14 |
| 25 | 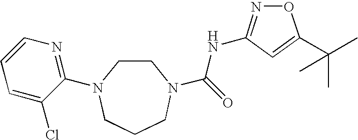 | 4-(3-Chloro-pyridin-2-yl)- [1,4]diazepane-1- carboxylic acid (5-tert- butyl-isoxazol-3-yl)-amide | 378 | 14 |
| 26 | 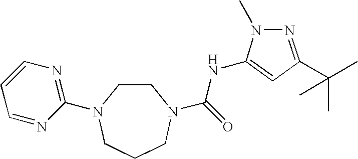 | 4-Pyrimidin-2-yl- [1,4]diazepane-1- carboxylic acid (5-tert- butyl-2-methyl-2H- pyrazol-3-yl)-amide | 358 | 14 |
| 27 | 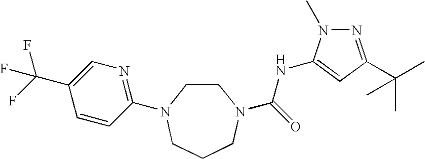 | 4-(5-Trifluoromethyl- pyridin-2-yl)- [1,4]diazepane-1- carboxylic acid (5-tert- butyl-2-methyl-2H- pyrazol-3-yl)-amide | 425 | 14 |
| 28 | 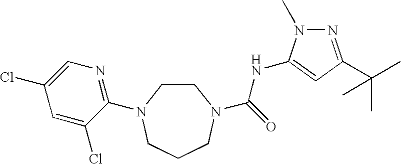 | 4-(3,5-Dichloro-pyridin-2- yl)-[1,4]diazepane-1- carboxylic acid (5-tert- butyl-2-methyl-2H- pyrazol-3-yl)-amide | 426 | 14 |
| 29 | 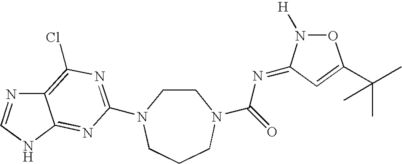 | 4-(6-Chloro-9H-purin-2- yl)-[1,4]diazepane-1- carboxylic acid [5-tert- butyl-isoxazol-(3E)- ylidene]-amide | 419 | 14 |
| 30 | 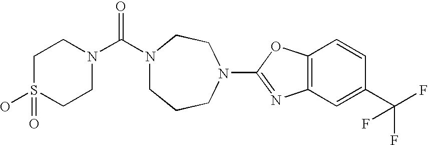 | (1,1-Dioxo-1□^{□}- thiomorpholin-4-yl)-[4-(5- trifluoromethyl- benzooxazol-2-yl)- [1,4]diazepan-1-yl]- methanone | 447 | 5 |
| 31 | 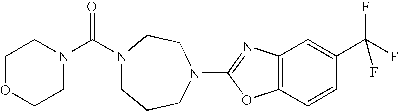 | Morpholin-4-yl-[4-(5- trifluoromethyl- benzooxazol-2-yl)- [1,4]diazepan-1-yl]- methanone | 399 | 12 |
| 32 | 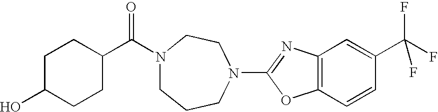 | (4-Hydroxy-cyclohexyl)- [4-(5-trifluoromethyl- [1,4]diazepan-1-yl]- methanone | 412 | 6 |
| 33 | 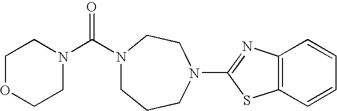 | (4-Benzothiazol-2-yl- [1,4]diazepan-1-yl)- morpholin-4-yl-methanone | 347 | 2 |
| 34 | 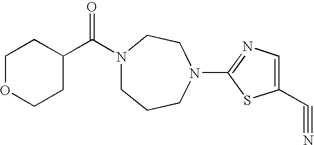 | 2-[4-(Tetrahydro-pyran-4- carbonyl)-[1,4]diazepan-1- yl]-thiazole-5-carbonitrile | 321 | 2 |
| 35 | 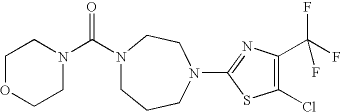 | [4-(5-Chloro-4- trifluoromethyl-thiazol-2- yl)-[1,4]diazepan-1-yl]- morpholin-4-yl-methanone | 399, 401 | 2 |
| 36 | 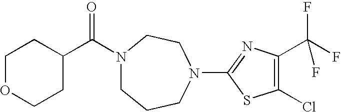 | [4-(5-Chloro-4- trifluoromethyl-thiazol-2- yl)-[1,4]diazepan-1-yl]- (tetrahydro-pyran-4-yl)- methanone | 398, 400 | 2 |
| 37 | 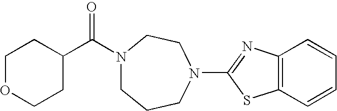 | (4-Benzothiazol-2-yl- [1,4]diazepan-1-yl)- (tetrahydro-pyran-4-yl)- methanone | 346 | 2 |
| 38 | 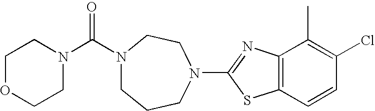 | [4-(5-Chloro-4-methyl- benzothiazol-2-yl)- [1,4]diazepan-1-yl]- morpholin-4-yl-methanone | 395, 397 | 2 |
| 39 | 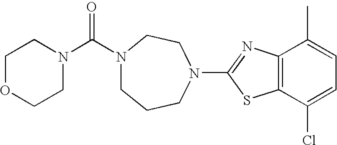 | [4-(7-Chloro-4-methyl- benzothiazol-2-yl)- [1,4]diazepan-1-yl]- morpholin-4-yl-methanone | 395, 397 | 2 |
| 40 | 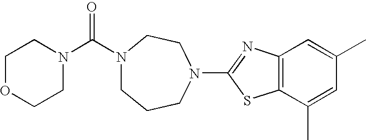 | [4-(5,7-Dimethyl- benzothiazol-2-yl)- [1,4]diazepan-1-yl]- morpholin-4-yl-methanone | 375 | 2 |
| 41 | 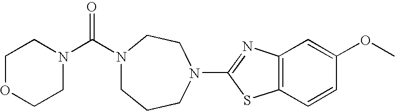 | [4-(5-Methoxy- benzothiazol-2-yl)- [1,4]diazepan-1-yl]- morpholin-4-yl-methanone | 377 | 2 |
| 42 | 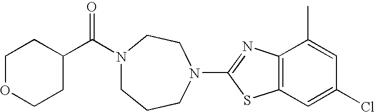 | [4-(6-Chloro-4-methyl- benzothiazol-2-yl)- [1,4]diazepan-1-yl]- (tetrahydro-pyran-4-yl)- methanone | 394, 396 | 2 |
| 43 | 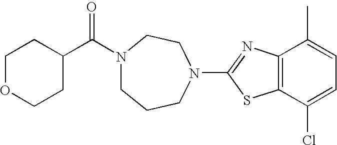 | [4-(7-Chloro-4-methyl- benzothiazol-2-yl)- [1,4]diazepan-1-yl]- (tetrahydro-pyran-4-yl)- methanone | 394, 397 | 2 |
| 44 | 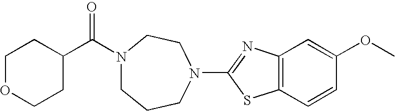 | [4-(5-Methoxy- benzothiazol-2-yl)- [1,4]diazepan-1-yl]- (tetrahydro-pyran-4-yl)- methanone | 376 | 2 |
| 45 | 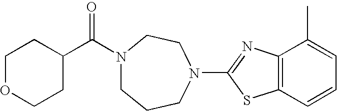 | [4-(4-Methyl-benzothiazol- 2-yl)-[1,4]diazepan-1-yl]- (tetrahydro-pyran-4-yl)- methanone | 360 | 2 |
| 46 | 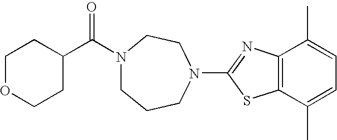 | [4-(4,7-Dimethyl- benzothiazol-2-yl)- [1,4]diazepan-1-yl]- (tetrahydro-pyran-4-yl)- methanone | 374 | 2 |
| 47 | 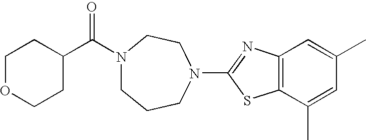 | [4-(5,7-Dimethyl- benzothiazol-2-yl)- [1,4]diazepan-1-yl]- (tetrahydro-pyran-4-yl)- methanone | 374 | 2 |
| 48 | 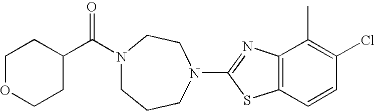 | [4-(5-Chloro-4-methyl- benzothiazol-2-yl)- [1,4]diazepan-1-yl]- (tetrahydro-pyran-4-yl)- methanone | 394, 396 | 2 |
| 49 | 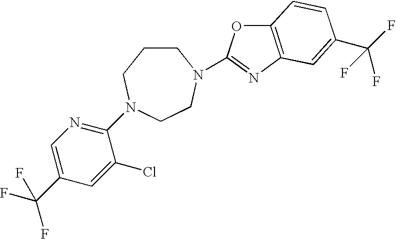 | 2-[4-(3-Chloro-5- trifluoromethyl-pyridin-2- yl)-[1,4]diazepan-1-yl]-5- trifluoromethyl- benzooxazole | 465, 467 | 7 |
| 50 | 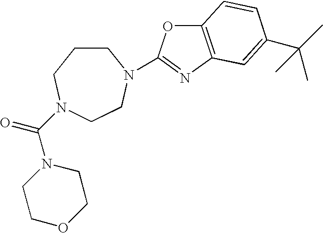 | [4-(5-tert-Butyl- benzooxazol-2-yl)- [1,4]diazepan-1-yl]- morpholin-4-yl-methanone | 387 | 9 |
| 51 | 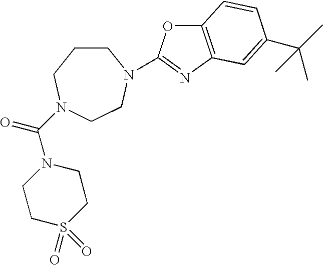 | [4-(5-tert-Butyl- benzooxazol-2-yl)- [1,4]diazepan-1-yl]-(1,1- dioxo-1λ⁶-thiomorpholin- 4-yl)-methanone | 435 | 10 |
| 52 | 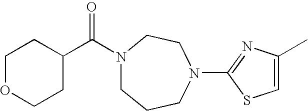 | [4-(4-Methyl-thiazol-2-yl)- [1,4]diazepan-1-yl]- (tetrahydro-pyran-4-yl)- methanone | 310 | 3 |
| 53 | 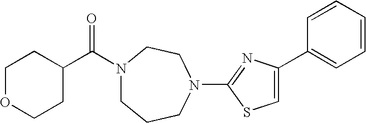 | [4-(4-Phenyl-thiazol-2-yl)- [1,4]diazepan-1-yl]- (tetrahydro-pyran-4-yl)- methanone | 372 | 3 |
| 54 | 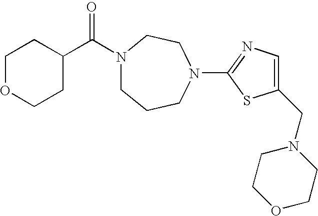 | [4-(5-Morpholin-4- ylmethyl-thiazol-2-yl)- [1,4]diazepan-1-yl]- (tetrahydro-pyran-4-yl)- methanone | 395, 417 (M + Na) | 3 |
| 55 | 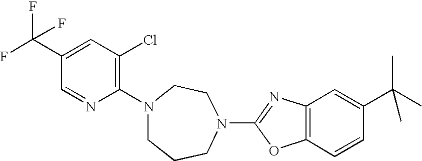 | 5-tert-Butyl-2-[4-(3-chloro- 5-trifluoromethyl-pyridin- 2-yl)-[1,4]diazepan-1-yl]- benzooxazole | 453, 455 | 11 |
| 56 | 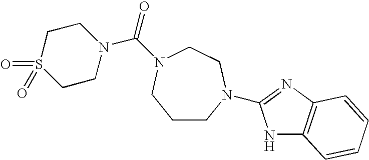 | [4-(1H-Benzoimidazol-2- yl)-[1,4]diazepan-1-yl]- (1,1-dioxo-1λ⁶- thiomorpholin-4-yl)- methanone | 378 | 17 |
| 57 | 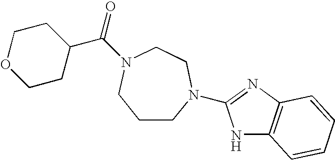 | [4-(1H-Benzoimidazol-2- yl)-[1,4]diazepan-1-yl]- (tetrahydro-pyran-4-yl)- methanone | 329 | 16 |
| 58 | 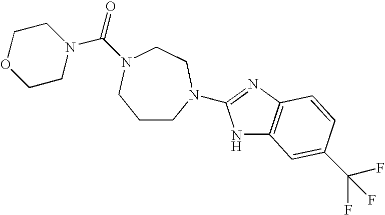 | Morpholin-4-yl-[4-(6- trifluoromethyl-1H- benzoimidazol-2-yl)- [1,4]diazepan-1-yl]- methanone | 398 | 18 |
| 59 | 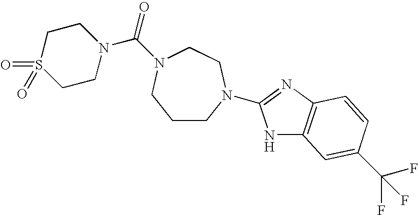 | (1,1-Dioxo-1λ⁶- thiomorpholin-4-yl)-[4-(6- trifluoromethyl-1H- benzoimidazol-2-yl)- [1,4]diazepan-1-yl]- methanone | 446 | 17 |
| 60 | 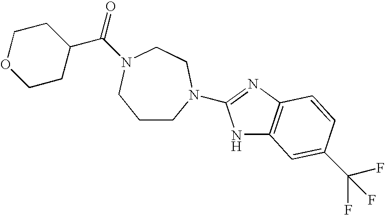 | (Tetrahydro-pyran-4-yl)-[4- (6-trifluoromethyl-1H- benzoimidazol-2-yl)- [1,4]diazepan-1-yl]- methanone | 397 | 16 |
| 61 | 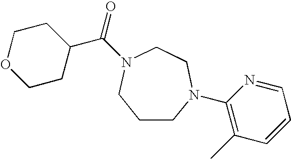 | [4-(3-Methyl-pyridin-2-yl)- [1,4]diazepan-1-yl]- (tetrahydro-pyran-4-yl)- methanone | 304 | 16 |
| 62 | 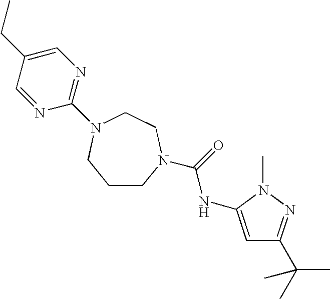 | 4-(5-Ethyl-pyrimidin-2-yl)- [1,4]diazepane-1- carboxylic acid (5-tert- butyl-2-methyl-2H- pyrazol-3-yl)-amide | 386 | 22 |
| 63 | 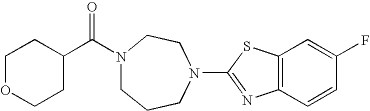 | [4-(6-Fluoro-benzothiazol- 2-yl)-[1,4]diazepan-1-yl]- (tetrahydro-pyran-4-yl)- methanone | 364 | 2 |
| 64 | 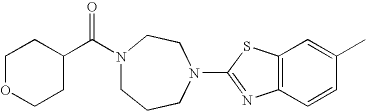 | [4-(6-Methyl-benzothiazol- 2-yl)-[1,4]diazepan-1-yl]- (tetrahydro-pyran-4-yl)- methanone | 360 | 2 |
| 65 | 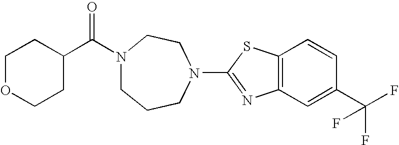 | (Tetrahydro-pyran-4-yl)-[4- (5-trifluoromethyl- benzoimidazol-2-yl)- [1,4]diazepan-1-yl]- methanone | 414 | 4 |
| 66 | 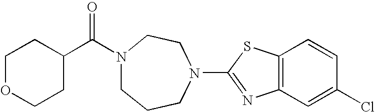 | [4-(5-Chloro-benzothiazol- 2-yl)-[1,4]diazepan-1-yl]- (tetrahydro-pyran-4-yl)- methanone | 380, 382 | 4 |
| 67 | 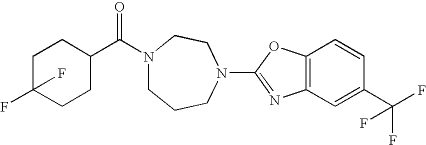 | (4,4-Difluoro-cyclohexyl)- [4-(5-trifluoromethyl- benzooxazol-2-yl)- [1,4]diazepan-1-yl]- methanone | 432 | 8 |
| 68 | 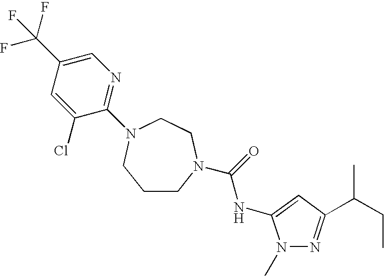 | 4-(3-Chloro-5- trifluoromethyl-pyridin-2- yl)-[1,4]diazepane-1- carboxylic acid (5-sec- butyl-2-methyl-2H- pyrazol-3-yl)-amide | 459/461 | 13 |
| 69 | 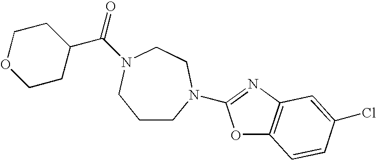 | [4-(5-Chloro-benzooxazol- 2-yl)-[1,4]diazepan-1-yl]- (tetrahydro-pyran-4-yl)- methanone | 364 | 19 |
| 70 | 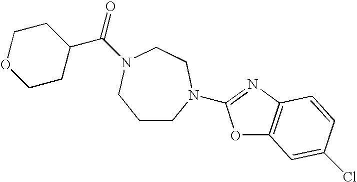 | [4-(6-Chloro-benzooxazol- 2-yl)-[1,4]diazepan-1-yl]- (tetrahydro-pyran-4-yl)- methanone | 364 | 19 |
| 71 | 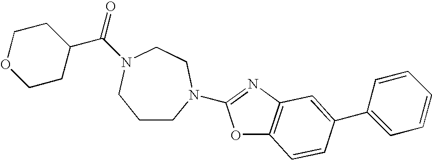 | [4-(5-Phenyl-benzooxazol- 2-yl)-[1,4]diazepan-1-yl]- (tetrahydro-pyran-4-yl)- methanone | 406 | 19 |
| 72 | 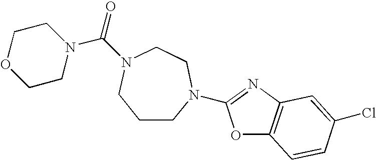 | [4-(5-Chloro-benzooxazol- 2-yl)-[1,4]diazepan-1-yl]- morpholin-4-yl-methanone | 365 | 20 |
| 73 | 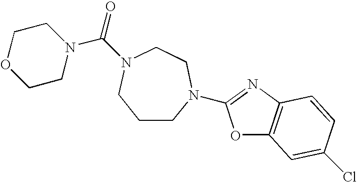 | [4-(6-Chloro-benzooxazol- 2-yl)-[1,4]diazepan-1-yl]- morpholin-4-yl-methanone | 365 | 20 |
| 74 | 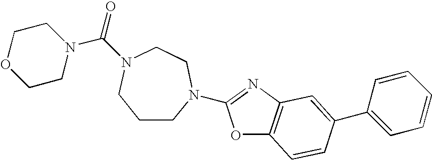 | Morpholin-4-yl-[4-(5- phenyl-benzooxazol-2-yl)- [1,4]diazepan-1-yl]- methanone | 407 | 20 |
| 75 | 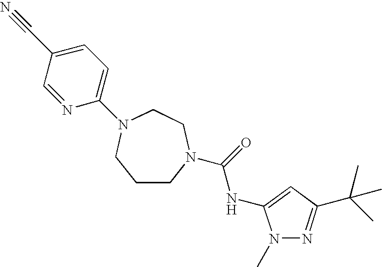 | 4-(5-Cyano-pyridin-2-yl)- [1,4]diazepane-1- carboxylic acid (5-tert!- butyl-2-methyl-2H!- pyrazol-3-yl)-amide | 382 | 23 |
| 76 | 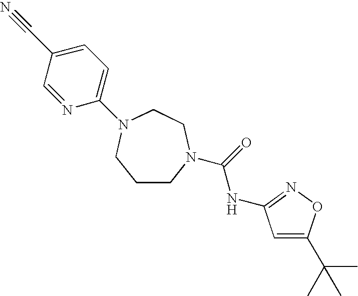 | 4-(5-Cyano-pyridin-2-yl)- [1,4]diazepane-1- carboxylic acid (5-tert!- butyl-isoxazol-3-yl)-amide | 369 | 23 |
| 77 | 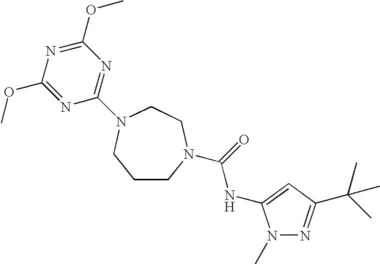 | 4-(4,6-Dimethoxy- [1,3,5]triazin-2-yl)- [1,4]diazepane-1- carboxylic acid (5-tert!- butyl-2-methyl-2H!- pyrazol-3-yl)-amide | 405 | 23 |
| 78 | 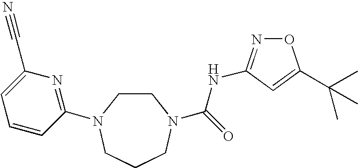 | 4-(6-Cyano-pyridin-2-yl)- [1,4]diazepane-1- carboxylic acid (5-tert!- butyl-isoxazol-3-yl)-amide | 369 | 23 |
| 79 | 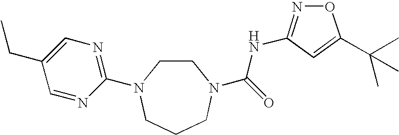 | 4-(5-Ethyl-pyrimidin-2-yl)- [1,4]diazepane-1- carboxylic acid (5-tert- butyl-isoxazol-3-yl)-amide | 373 | 23 |
| 80 | 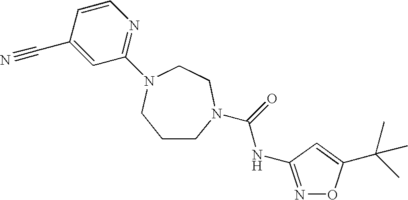 | 4-(4-Cyano-pyridin-2-yl)- [1,4]diazepane-1- carboxylic acid (5-tert!- butyl-isoxazol-3-yl)-amide | 369 | 23 |
| 81 | 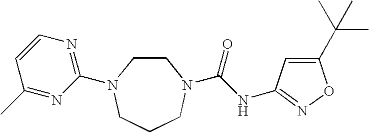 | 4-(4-Methyl-pyrimidin-2-yl)- [1,4]diazepane-1- carboxylic acid (5-tert!- butyl-isoxazol-3-yl)-amide | 359 | 23 |
| 82 | 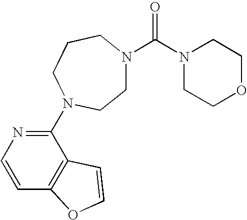 | (4-Furo[3,2-c!]pyridin-4- yl-[1,4]diazepan-1-yl)- morpholin-4-yl-methanone | 331 | 24 |
| 83 | 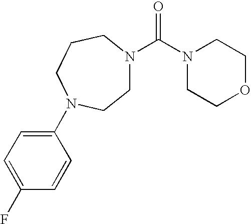 | [4-(4-Fluoro-phenyl- [1,4]diazepan-1-yl]- morpholin-4-yl-methanone | 308 | 24 |
| 84 | 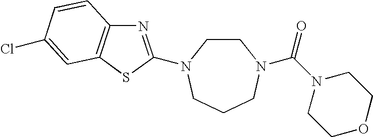 | [4-(6-Chloro-benzothiazol- 2-yl)-[1,4]diazepan-1-yl]- morpholin-4-yl-methanone | 381 | 24 |
| 85 | 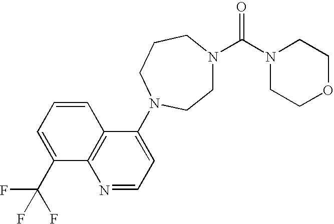 | Morpholin-4-yl-[4-(8- trifluoromethyl-quinolin-4- yl)-[1,4]diazepan-1-yl]- methanone | 409 | 24 |
| 86 | 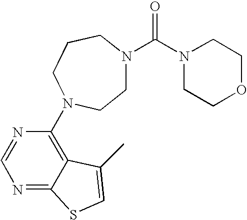 | [4-(5-Methyl-thieno[2,3- d]pyrimidin-4-yl)- [1,4]diazepan-1-yl]- morpholin-4-yl-methanone | 362 | 24 |
| 87 | 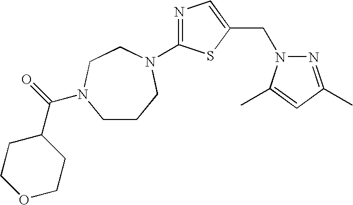 | {4-[5-(3,5-Dimethyl- pyrazol-1-ylmethyl)- thiazol-2-yl]-[1,4]diazepan- 1-yl}-(tetrahydro-pyran-4- yl)-methanone | 404 | 3 |
| 88 | 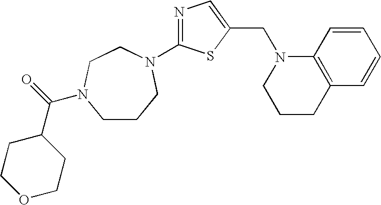 | {4-[5-(3,4-Dihydro-2H- quinolin-1-ylmethyl)- thiazol-2-yl]-[1,4]diazepan- 1-yl}-(tetrahydro-pyran-4- yl)-methanone | 441 | 3 |
| 89 | 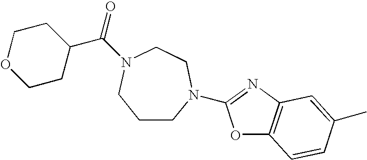 | [4-(5-Methyl-benzooxazol- 2-yl)-[1,4]diazepan-1-yl]- (tetrahydro-pyran-4-yl)- methanone | 344 | 19 |
| 90 | 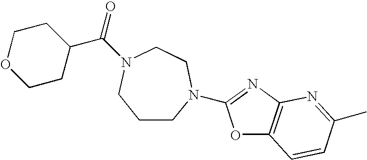 | [4-(5-Methyl-oxazolo[4,5- b]pyridin-2-yl)- [1,4]diazepan-1-yl]- (tetrahydro-pyran-4-yl)- methanone | 345 | 19 |

Assessment of Biological Properties

The biological properties of the compounds of the formula I were assessed using the assays described below.

A. Human CB1 and CB2 Receptor Binding:

Experimental Method:

CB2 membranes were purchased and made from HEK293 EBNA cells stably transfected with human CB2 receptor cDNA (Perkin Elmer Life and Analytical Sciences). CB1 membranes were isolated from HEK cells stably co-transfected with human CB1 receptor and Gα16 cDNA's. The membrane preparation was bound to scintillation beads (Ysi-Poly-L-lysine SPA beads, GE Healthcare) for 4 hours at room temperature in assay buffer containing 50 mM Tris, pH 7.5, 2.5 mM EDTA, 5 mM MgCl₂, 0.8% fatty acid free Bovine Serum Albumin. Unbound membrane was removed by washing in assay buffer. Membrane-bead mixture was added to 96-well assay plates in the amounts of 15 ug membrane per well (CB2) or 2.5 ug per well (CB1) and 1 mg SPA bead per well. Compounds were added to the membrane-bead mixture in dose-response concentrations ranging from 1×10⁻⁵ M to 1×10⁻¹⁰ M with 0.25% DMSO, final. The competition reaction was initiated with the addition of ³H-CP55940 (Perkin Elmer Life and Analytical Sciences) at a final concentration of 1.5 nM (CB2) or 2.5 nM (CB1). The reaction was incubated at room temperature for 18 hours and read on TopCount NXT plate reader. Total and non-specific binding was determined in the absence and presence of 1.25 uM Win 55212 (Sigma). IC50 values for each compound were calculated as the concentration of compound that inhibits the specific binding of the radioactively labeled ligand to the receptor by 50% using the XLFit 4.1 four parameter logistic model. IC50 values were converted to inhibition constant (Ki) values using Cheng-Prusoff equation.

B. CB2R Mediated Modulation of cAMP Synthesis:

Compounds of the invention were evaluated for their CB2 agonist or inverse agonistic activity in accordance with the following experimental method. Compounds which were shown to bind to CB2 by the binding assay described above but which were not shown to exhibit CB2R-mediated modulation of cAMP synthesis by this assay were presumed to be CB2 antagonists.

Experimental Method:

CHO cells expressing human CB2R (Euroscreen) were plated at a density of 5000 cells per well in 384 well plates and incubated overnight at 37° C. After removing the media, the cells were treated with test compounds diluted in stimulation buffer containing 1 mM IBMX, 0.25% BSA and 10 uM Forskolin. The assay was incubated for 30 minutes at 37° C. Cells were lysed and the cAMP concentration was measured using DiscoverX-XS cAMP kit, following the manufacturer's protocol. In this setting, agonists will decrease forskolin induced production of cAMP while inverse agonists will further increase forskolin induced production of cAMP. EC50 of agonists were calculated as follows. The maximal amount of cAMP produced by forskolin compared to the level of cAMP inhibited by 1 uM CP55940 is defined as 100%. The EC50 value of each test compound was determined as the concentration at which 50% of the forskolin-stimulated cAMP synthesis was inhibited. Data was analyzed using a four-parameter logistic model. (Model 205 of XLfit 4.0).

C. CB1R Mediated Modulation of cAMP Synthesis:

Compounds of the invention were evaluated for their CB1 agonist or inverse agonistic activity in accordance with the following experimental method. Compounds which were shown to bind to CB1 by the binding assay described above but which were not shown to exhibit CB1R-mediated modulation of cAMP synthesis by this assay were presumed to be CB1 antagonists.

Experimental Method:

CHO cells expressing human CB1R (Euroscreen) were plated at a density of 5000 cells per well in 384 well plates and incubated overnight at 37° C. After removing the media, the cells were treated with test compounds diluted in stimulation buffer containing 1 mM IBMX, 0.25% BSA and 10 uM Forskolin. The assay was incubated for 30 minutes at 37° C. Cells were lysed and the cAMP concentration was measured using DiscoverX-XS cAMP kit, following the manufacturer's protocol. In this setting, agonists will decrease forskolin induced production of cAMP while inverse agonists will further increase forskolin induced production of cAMP. EC50 of agonists were calculated as follows. The maximal amount of cAMP produced by forskolin compared to the level of cAMP inhibited by 1 uM CP55940 is defined as 100%. The EC50 value of each test compound was determined as the concentration at which 50% of the forskolin-stimulated cAMP synthesis was inhibited. Data was analyzed using a four-parameter logistic model. (Model 205 of XLfit 4.0).

Compounds Having Agonist Activity

Through the use of the above described assays compounds were found to exhibit agonistic activity and thus to be particularly well suited for the treatment of pain as well as for the treatment of inflammation.

Therapeutic Use

As can be demonstrated by the assays described above, the compounds of the invention are useful in modulating the CB2 receptor function. By virtue of this fact, these compounds have therapeutic use in treating disease-states and conditions mediated by the CB2 receptor function or that would benefit from modulation of the CB2 receptor function.

As the compounds of the invention modulate the CB2 receptor function, they have very useful anti-inflammatory and immune-suppressive activity and they can be used in patients as drugs, particularly in the form of pharmaceutical compositions as set forth below, for the treatment of disease-states and conditions.

As noted before, those compounds which are CB2 agonists can also be employed for the treatment of pain.

The agonist, antagonist and inverse agonist compounds according to the invention can be used in patients as drugs for the treatment of the following disease-states or indications that are accompanied by inflammatory processes:
(i) Lung diseases: e.g. asthma, bronchitis, allergic rhinitis, emphysema, adult respiratory distress syndrome (ARDS), pigeon fancier's disease, farmer's lung, chronic obstructive pulmonary disease (COPD), asthma including allergic asthma (atopic or non-atopic) as well as exercise-induced bronchoconstriction, occupational asthma, viral- or bacterial exacerbation of asthma, other non-allergic asthmas and “wheezy-infant syndrome”, pneumoconiosis, including aluminosis, anthracosis, asbestosis, chalicosis, ptilosis, siderosis, silicosis, tabacosis and byssinosis;
(ii) Rheumatic diseases or autoimmune diseases or musculoskeletal diseases: all forms of rheumatic diseases, especially rheumatoid arthritis, acute rheumatic fever, and polymyalgia rheumatica; reactive arthritis; rheumatic soft tissue diseases; inflammatory soft tissue diseases of other genesis; arthritic symptoms in degenerative joint diseases (arthroses); tendinitis, bursitis, osteoarthritis, traumatic arthritis; collagenoses of any genesis, e.g., systemic lupus erythematosus, scleroderma, polymyositis, dermatomyositis, Sjögren syndrome, Still disease, Felty syndrome; and osteoporosis and other bone resorption diseases;
(iii) Allergic diseases: all forms of allergic reactions, e.g., angioneurotic edema, hay fever, insect bites, allergic reactions to drugs, blood derivatives, contrast agents, etc., anaphylactic shock (anaphylaxis), urticaria, angioneurotic edema, and contact dermatitis;
(iv) Vascular diseases: panarteritis nodosa, polyarteritis nodosa, periarteritis nodosa, arteritis temporalis, Wegner granulomatosis, giant cell arthritis, atherosclerosis, reperfusion injury and erythema nodosum;
(v) Dermatological diseases: e.g. dermatitis, psoriasis; sunburn, burns, eczema;
(vi) Renal diseases: e.g. nephrotic syndrome; and all types of nephritis, e.g., glomerulonephritis; pancreatits;
(vii) Hepatic diseases: e.g. acute liver cell disintegration; acute hepatitis of various genesis, e.g., viral, toxic, drug-induced; and chronically aggressive and/or chronically intermittent hepatitis;
(viii) Gastrointestinal diseases: e.g. inflammatory bowel diseases, irritable bowel syndrome, regional enteritis (Crohns disease), colitis ulcerosa; gastritis; aphthous ulcer, celiac disease, regional ileitis, gastroesophageal reflux disease;
(ix) Neuroprotection: e.g. in the treatment of neurodegeneration following stroke; cardiac arrest; pulmonary bypass; traumatic brain injury; spinal cord injury or the like;
(x) Eye diseases: allergic keratitis, uveitis, or iritis; conjunctivitis; blepharitis; neuritis nervi optici; choroiditis; glaucoma and sympathetic ophthalmia;
(xi) Diseases of the ear, nose, and throat (ENT) area: e.g. tinnitus; allergic rhinitis or hay fever; otitis externa; caused by contact eczema, infection, etc.; and otitis media;
(xii) Neurological diseases: e.g. brain edema, particularly tumor-related brain edema; multiple sclerosis; acute encephalomyelitis; meningitis; acute spinal cord injury; trauma; dementia, particularly degenerative dementia (including senile dementia, Alzheimer's disease; Parkinson's disease and Creutzfeldt-Jacob disease; Huntington's chorea, Pick's disease; motor neuron disease), vascular dementia (including multi-infarct dementia) as well as dementia associated with intracranial space occupying lesions; infections and related conditions (including HIV infection); Guillain-Barre syndrome; myasthenia gravis, stroke; and various forms of seizures, e.g., nodding spasms;
(xiii) Blood diseases: acquired hemolytic anemia; aplastic anemia, and idiopathic thrombocytopenia;
(xiv) Tumor diseases: acute lymphatic leukemia; Hodgkin's disease, malignant lymphoma; lymphogranulomatoses; lymphosarcoma; solid malignant tumors; extensive metastases;
(xv) Endocrine diseases: endocrine opthalmopathy; endocrine orbitopathia; thyrotoxic crisis; Thyroiditis de Quervain; Hashimoto thyroiditis; Morbus Basedow; granulomatous thyroiditis; struma lymphomatosa; and Graves disease; type I diabetes (insulin-dependent diabetes);
(xvi) Organ and tissue transplantations and graft-versus-host diseases;
(xvii) Severe states of shock, e.g., septic shock, anaphylactic shock, and systemic inflammatory response syndrome (SIRS);
(xviii) Acute pain such as dental pain, perioperative, post-operative pain, traumatic pain, muscle pain, pain in burned skin, sun burn, trigeminal neuralgia, sun burn; spasm of the gastrointestinal tract or uterus, colics;
(xix) Visceral pain such as pain associated with chronic pelvic pain, pancreatitis, peptic ulcer, interstitial cystitis, renal colic, angina, dysmenorrhoea, menstruation, gynaecological pain, irritable bowel syndrome (IBS), non-ulcer dyspepsia, non-cardiac chest pain, myocardial ischemia;
(xx) Neuropathic pain such as low back pain, non-herpetic neuralgia, post herpetic neuralgia, diabetic neuropathy, nerve injury, acquired immune deficiency syndrome (AIDS) related neuropathic pain, head trauma, painful traumatic mononeuropathy, toxin and chemotherapy induced pain, phantom limb pain, painful polyneuropathy, thalamic pain syndrome, post-stroke pain, central nervous system injury, post surgical pain, stump pain, repetitive motion pain, pain induced by post mastectomy syndrome, multiple sclerosis, root avulsions, postthoracotomy syndrome, neuropathic pain associated hyperalgesia and allodynia.
(xxi) Inflammatory/nociceptive pain induced by or associated with disorders such as osteoarthritis, rheumatoid arthritis, rheumatic disease, teno-synovitis, gout, vulvodynia, myofascial pain (muscular injury, fibromyalgia), tendonitis, osteoarthritis, juvenile arthritis, spondylitis, gouty arthritis, psoriatic arthritis, muscoskeletal pain, fibromyalgia, sprains and strains, sympathetically maintained pain, myositis, pain associated with migraine, toothache, influenza and other viral infections such as the common cold, rheumatic fever, systemic lupus erythematosus;
(xxii) Cancer pain induced by or associated with tumors such as lymphatic leukemia; Hodgkin's disease, malignant lymphoma; lymphogranulomatoses; lymphosarcoma; solid malignant tumors; extensive metastases;
(xxiii) Headache such as cluster headache, migraine with and without aura, tension type headache, headache with different origins, headache disorders including prophylactic and acute use;
(xxiv) various other disease-states or conditions including, restenosis following percutaneous transluminal coronary angioplasty, acute and chronic pain, atherosclerosis, reperfusion injury, congestive heart failure, myocardial infarction, thermal injury, multiple organ injury secondary to trauma, necrotizing enterocolitis and syndromes associated with hemodialysis, leukopheresis, and granulocyte transfusion, sarcoidosis, gingivitis, pyrexia, edema resulting from trauma associated with bums, sprains or fracture, cerebral oedema and angioedema, Diabetes such as diabetic vasculopathy, diabetic neuropathy, diabetic retinopathy, post capillary resistance or diabetic symptoms associated with insulitis (e.g. hypergiycemia, diuresis, proteinuria and increased nitrite and kallikrein urinary excretion).

Other indications include: epilepsy, septic shock e.g. as antihypovolemic and/or antihypotensive agents, cancer, sepsis, osteoporosis, benign prostatic hyperplasia and hyperactive bladder, pruritis, vitiligo, general gastrointestinal disorders, disturbances of visceral motility at respiratory, genitourinary, gastrointestinal or vascular regions, wounds, burns, tissue damage and postoperative fever, syndromes associated with Itching.

Besides being useful for human treatment, these compounds are also useful for veterinary treatment of companion animals, exotic animals and farm animals, including mammals, rodents, and the like.

For treatment of the above-described diseases and conditions, a therapeutically effective dose will generally be in the range from about 0.01 mg to about 100 mg/kg of body weight per dosage of a compound of the invention; preferably, from about 0.1 mg to about 20 mg/kg of body weight per dosage. For example, for administration to a 70 kg person, the dosage range would be from about 0.7 mg to about 7000 mg per dosage of a compound of the invention, preferably from about 7.0 mg to about 1400 mg per dosage. Some degree of routine dose optimization may be required to determine an optimal dosing level and pattern. The active ingredient may be administered from 1 to 6 times a day.

General Administration and Pharmaceutical Compositions

When used as pharmaceuticals, the compounds of the invention are typically administered in the form of a pharmaceutical composition. Such compositions can be prepared using procedures well known in the pharmaceutical art and comprise at least one compound of the invention. The compounds of the invention may also be administered alone or in combination with adjuvants that enhance stability of the compounds of the invention, facilitate administration of pharmaceutical compositions containing them in certain embodiments, provide increased dissolution or dispersion, increased inhibitory activity, provide adjunct therapy, and the like. The compounds according to the invention may be used on their own or in conjunction with other active substances according to the invention, optionally also in conjunction with other pharmacologically active substances. In general, the compounds of this invention are administered in a therapeutically or pharmaceutically effective amount, but may be administered in lower amounts for diagnostic or other purposes.

Administration of the compounds of the invention, in pure form or in an appropriate pharmaceutical composition, can be carried out using any of the accepted modes of administration of pharmaceutical compositions. Thus, administration can be, for example, orally, buccally (e.g., sublingually), nasally, parenterally, topically, transdermally, vaginally, or rectally, in the form of solid, semi-solid, lyophilized powder, or liquid dosage forms, such as, for example, tablets, suppositories, pills, soft elastic and hard gelatin capsules, powders, solutions, suspensions, or aerosols, or the like, preferably in unit dosage forms suitable for simple administration of precise dosages. The pharmaceutical compositions will generally include a conventional pharmaceutical carrier or excipient and a compound of the invention as the/an active agent, and, in addition, may include other medicinal agents, pharmaceutical agents, carriers, adjuvants, diluents, vehicles, or combinations thereof. Such pharmaceutically acceptable excipients, carriers, or additives as well as methods of making pharmaceutical compositions for various modes or administration are well-known to those of skill in the art. The state of the art is evidenced, e.g., by Remington: The Science and Practice of Pharmacy, 20th Edition, A. Gennaro (ed.), Lippincott Williams & Wilkins, 2000; Handbook of Pharmaceutical Additives, Michael & Irene Ash (eds.), Gower, 1995; Handbook of Pharmaceutical Excipients, A. H. Kibbe (ed.), American Pharmaceutical Ass'n, 2000; H. C. Ansel and N. G. Popovish, Pharmaceutical Dosage Forms and Drug Delivery Systems, 5th ed., Lea and Febiger, 1990; each of which is incorporated herein by reference in their entireties to better describe the state of the art.

As one of skill in the art would expect, the forms of the compounds of the invention utilized in a particular pharmaceutical formulation will be selected (e.g., salts) that possess suitable physical characteristics (e.g., water solubility) that is required for the formulation to be efficacious.

Pharmaceutical compositions suitable for buccal (sub-lingual) administration include lozenges comprising a compound of the present invention in a flavored base, usually sucrose, and acacia or tragacanth, and pastilles comprising the compound in an inert base such as gelatin and glycerin or sucrose and acacia.

Pharmaceutical compositions suitable for parenteral administration comprise sterile aqueous preparations of a compound of the present invention. These preparations are preferably administered intravenously, although administration can also be effected by means of subcutaneous, intramuscular, or intradermal injection. Injectable pharmaceutical formulations are commonly based upon injectable sterile saline, phosphate-buffered saline, oleaginous suspensions, or other injectable carriers known in the art and are generally rendered sterile and isotonic with the blood. The injectable pharmaceutical formulations may therefore be provided as a sterile injectable solution or suspension in a nontoxic parenterally acceptable diluent or solvent, including 1,3-butanediol, water, Ringer's solution, isotonic sodium chloride solution, fixed oils such as synthetic mono- or diglycerides, fatty acids such as oleic acid, and the like. Such injectable pharmaceutical formulations are formulated according to the known art using suitable dispersing or setting agents and suspending agents. Injectable compositions will generally contain from 0.1 to 5% w/w of a compound of the invention.

Solid dosage forms for oral administration of the compounds include capsules, tablets, pills, powders, and granules. For such oral administration, a pharmaceutically acceptable composition containing a compound(s) of the invention is formed by the incorporation of any of the normally employed excipients, such as, for example, pharmaceutical grades of mannitol, lactose, starch, pregelatinized starch, magnesium stearate, sodium saccharine, talcum, cellulose ether derivatives, glucose, gelatin, sucrose, citrate, propyl gallate, and the like. Such solid pharmaceutical formulations may include formulations, as are well-known in the art, to provide prolonged or sustained delivery of the drug to the gastrointestinal tract by any number of mechanisms, which include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form.

Liquid dosage forms for oral administration of the compounds include emulsions, microemulsions, solutions, suspensions, syrups, and elixirs, optionally containing pharmaceutical adjuvants in a carrier, such as, for example, water, saline, aqueous dextrose, glycerol, ethanol and the like. These compositions can also contain additional adjuvants such as wetting, emulsifying, suspending, sweetening, flavoring, and perfuming agents.

Topical dosage forms of the compounds include ointments, pastes, creams, lotions, gels, powders, solutions, sprays, inhalants, eye ointments, eye or ear drops, impregnated dressings and aerosols, and may contain appropriate conventional additives such as preservatives, solvents to assist drug penetration and emollients in ointments and creams. Topical application may be once or more than once per day depending upon the usual medical considerations. Furthermore, preferred compounds for the present invention can be administered in intranasal form via topical use of suitable intranasal vehicles. The formulations may also contain compatible conventional carriers, such as cream or ointment bases and ethanol or oleyl alcohol for lotions. Such carriers may be present as from about 1% up to about 98% of the formulation, more usually they will form up to about 80% of the formulation.

Transdermal administration is also possible. Pharmaceutical compositions suitable for transdermal administration can be presented as discrete patches adapted to remain in intimate contact with the epidermis of the recipient for a prolonged period of time. To be administered in the form of a transdermal delivery system, the dosage administration will, of course, be continuous rather than intermittent throughout the dosage regimen. Such patches suitably contain a compound of the invention in an optionally buffered, aqueous solution, dissolved and/or dispersed in an adhesive, or dispersed in a polymer. A suitable concentration of the active compound is about 1% to 35%, preferably about 3% to 15%.

For administration by inhalation, the compounds of the invention are conveniently delivered in the form of an aerosol spray from a pump spray device not requiring a propellant gas or from a pressurized pack or a nebulizer with the use of a suitable propellant, e.g., dichlorodifluoromethane, trichlorofluoromethane, dichlorotetrafluoroethane, tetrafluoroethane, heptafluoropropane, carbon dioxide, or other suitable gas. In any case, the aerosol spray dosage unit may be determined by providing a valve to deliver a metered amount so that the resulting metered dose inhaler (MDI) is used to administer the compounds of the invention in a reproducible and controlled way. Such inhaler, nebulizer, or atomizer devices are known in the prior art, for example, in PCT International Publication Nos. WO 97/12687 (particularly FIG. 6 thereof, which is the basis for the commercial RESPIMAT® nebulizer); WO 94/07607; WO 97/12683; and WO 97/20590, to which reference is hereby made and each of which is incorporated herein by reference in their entireties.

Rectal administration can be effected utilizing unit dose suppositories in which the compound is admixed with low-melting water-soluble or insoluble solids such as fats, cocoa butter, glycerinated gelatin, hydrogenated vegetable oils, mixtures of polyethylene glycols of various molecular weights, or fatty acid esters of polyethylene glycols, or the like. The active compound is usually a minor component, often from about 0.05 to 10% by weight, with the remainder being the base component.

In all of the above pharmaceutical compositions, the compounds of the invention are formulated with an acceptable carrier or excipient. The carriers or excipients used must, of course, be acceptable in the sense of being compatible with the other ingredients of the composition and must not be deleterious to the patient. The carrier or excipient can be a solid or a liquid, or both, and is preferably formulated with the compound of the invention as a unit-dose composition, for example, a tablet, which can contain from 0.05% to 95% by weight of the active compound. Such carriers or excipients include inert fillers or diluents, binders, lubricants, disintegrating agents, solution retardants, resorption accelerators, absorption agents, and coloring agents. Suitable binders include starch, gelatin, natural sugars such as glucose or β-lactose, corn sweeteners, natural and synthetic gums such as acacia, tragacanth or sodium alginate, carboxymethylcellulose, polyethylene glycol, waxes, and the like. Lubricants include sodium oleate, sodium stearate, magnesium stearate, sodium benzoate, sodium acetate, sodium chloride, and the like. Disintegrators include starch, methyl cellulose, agar, bentonite, xanthan gum, and the like.

Pharmaceutically acceptable carriers and excipients encompass all the foregoing additives and the like.

1. A compound of the formula 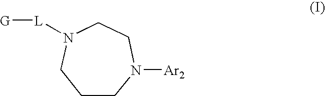
wherein:
L is a bond, —C(O), —CH₂—C(O)— or —NH—C(O)—;
G is chosen from a C1-10 alkyl branched or unbranched wherein one or more methylene groups may be replaced by NH, O or S(O)ₘ, Ar₁(CH₂)₀₋₂— and Ar₁;
Ar₁ is chosen from carbocycle, benzoxazolyl, benzothiazolyl, benzimidazolyl, tetrahydropyranyl, dioxanyl, tetrahydrofuranyl, oxazolyl, isoxazolyl, thiazolyl, pyrazolyl, pyrrolyl, imidazolyl, thienyl, thiadiazolyl, thiomorpholinyl, 1,1-dioxo-1λ⁶-thiomorpholinyl, morpholinyl, pyridinyl, pyrimidinyl, pyridazinyl, pyrazinyl, triazinyl, pyrrolidinyl, piperidinyl and piperazinyl each optionally substituted by one to three R₁;
Ar₂ is chosen from carbocycle, benzoxazolyl, benzothiazolyl, benzimidazolyl, purinyl, quinolinyl, isoquinolinyl, quinazolinyl, indazolyl, thieno[2,3-d]pyrimidinyl, indolyl, isoindolyl, benzofuranyl, benzopyranyl, benzodioxolyl, tetrahydropyranyl, dioxanyl, tetrahydrofuranyl, oxazolyl, isoxazolyl, thiazolyl, pyrazolyl, pyrrolyl, imidazolyl, thienyl, thiadiazolyl, thiomorpholinyl, thiomorpholinyl, morpholinyl, pyridinyl, pyrimidinyl, pyridazinyl, pyrazinyl, triazinyl, pyrrolidinyl, piperidinyl and piperazinyl each optionally substituted by one to three R₂;
m is 0, 1 or 2;
R₁ is C₁₋₁₀ alkyl, halogen, oxo (═O), C₁₋₁₀ alkoxy, carbocycle, C₁-C₆ acyl, C₁-C₆ acylamino, hydroxyl, amino, nitro, cyano, C₁-C₄ mono- or di-alkylaminoC₀-C₄alkyl and phenyl, each R₁ where possible is optionally partially or fully halogenated;
R₂ is C₁-C₁₀ alkyl, halogen, carbocycle, cyano or C₁₋₁₀ alkoxy, each R₂ is optionally partially or fully halogenated and optionally substituted by carbocycle-C₀₋₂ alkyl or a heteroring-C₀₋₂ alkyl wherein the heteroring is chosen from
benzoxazolyl, benzothiazolyl, benzimidazolyl, purinyl, quinolinyl, dihydro-2H-quinolinyl, isoquinolinyl, quinazolinyl, indazolyl, thieno[2,3-d]pyrimidinyl, indolyl, isoindolyl, benzofuranyl, benzopyranyl, benzodioxolyl, tetrahydropyranyl, dioxanyl, tetrahydrofuranyl, oxazolyl, isoxazolyl, thiazolyl, pyrazolyl, pyrrolyl, imidazolyl, thienyl, thiadiazolyl, thiomorpholinyl, thiomorpholinyl, morpholinyl, pyridinyl, pyrimidinyl, pyridazinyl, pyrazinyl, triazinyl, pyrrolidinyl, piperidinyl and piperazinyl each R₂ carbocycle or heteroring optionally independently substituted with 1 to 3 substituents chosen from C₁-C₆ alkyl, C₁-C₆ alkoxy, C₁-C₆ acyl, C₁-C₆ acylamino, C₃-C₆ cycloalkyl, phenoxy, halogen, hydroxyl, amino, nitro, cyano, C₁-C₄ mono- or di-alkylaminoC₀-C₄alkyl and phenyl;
or a pharmaceutically acceptable salt thereof.

2. The compound according to claim 1 and wherein,
Ar₁ is chosen from benzoxazolyl, benzothiazolyl, benzimidazolyl, cyclohexyl, cyclopentyl, phenyl, tetrahydropyranyl, dioxanyl, tetrahydrofuranyl, oxazolyl, isoxazolyl, thiazolyl, pyrazolyl, pyrrolyl, imidazolyl, thienyl, thiadiazolyl, thiomorpholinyl, 1,1-Dioxo-1λ⁶-thiomorpholinyl, morpholinyl, pyridinyl, pyrimidinyl, pyridazinyl, pyrazinyl, triazinyl, pyrrolidinyl, piperidinyl and piperazinyl each optionally substituted by one to three R₁;
Ar₂ is chosen from phenyl, benzoxazolyl, benzothiazolyl, benzimidazolyl, purinyl, quinolinyl, isoquinolinyl, thieno[2,3-d]pyrimidinyl, oxazolyl, isoxazolyl, thiazolyl, pyrazolyl, thiadiazolyl, pyridinyl, pyrimidinyl, pyridazinyl, pyrazinyl, triazinyl, piperidinyl and piperazinyl each optionally substituted by one to three R₂;
R₁ is C₁₋₁₀ alkyl, halogen or oxo;
R₂ is C₁-C₁₀ alkyl, halogen, carbocycle, cyano or C₁₋₁₀ alkoxy, each R₂ is optionally partially or fully halogenated and optionally substituted by phenyl, benzyl or a heteroring-C₀₋₂ alkyl wherein the heteroring is chosen from
quinolinyl, dihydro-2H-quinolinyl, isoquinolinyl, oxazolyl, isoxazolyl, thiazolyl, pyrazolyl, pyrrolyl, imidazolyl, thiadiazolyl, thiomorpholinyl, thiomorpholinyl, morpholinyl, pyridinyl, pyrimidinyl, pyridazinyl, pyrazinyl, triazinyl, pyrrolidinyl, piperidinyl and piperazinyl each R₂ ring is optionally independently substituted with 1 to 3 substituents chosen from C₁-C₆ alkyl, C₁-C₆ alkoxy, C₁-C₆ acyl, C₁-C₆ acylamino, C₃-C₆ cycloalkyl, phenoxy, halogen, hydroxyl, amino, nitro, cyano, C₁-C₄ mono- or di-alkylaminoC₀-C₄alkyl and phenyl.

3. The compound according to claim 2 and wherein,
R₁ is C₁₋₆ alkyl, halogen or oxo;
R₂ is C₁-C₁₀ alkyl, halogen, phenyl, cyano or C₁₋₁₀ alkoxy, each R₂ is optionally partially or fully halogenated and optionally substituted by quinolinyl, dihydro-2H-quinolinyl, morpholinyl, each R₂ ring optionally independently substituted with 1 to 3 substituents chosen from C₁-C₆ alkyl, C₁-C₆ alkoxy, halogen and cyano.

4. The compound according to claim 3 and wherein,
G is chosen from 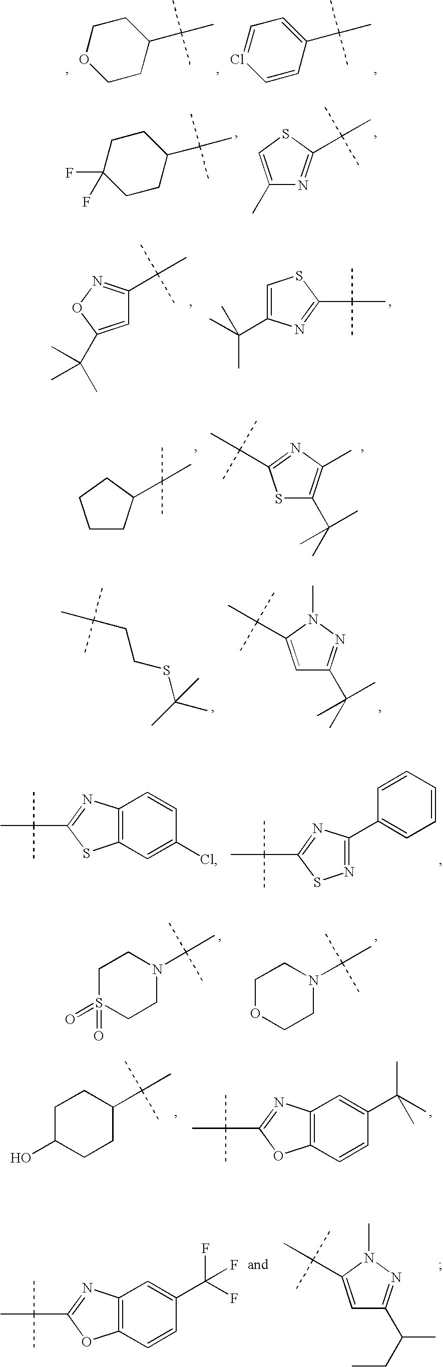
Ar₂ is chosen from:

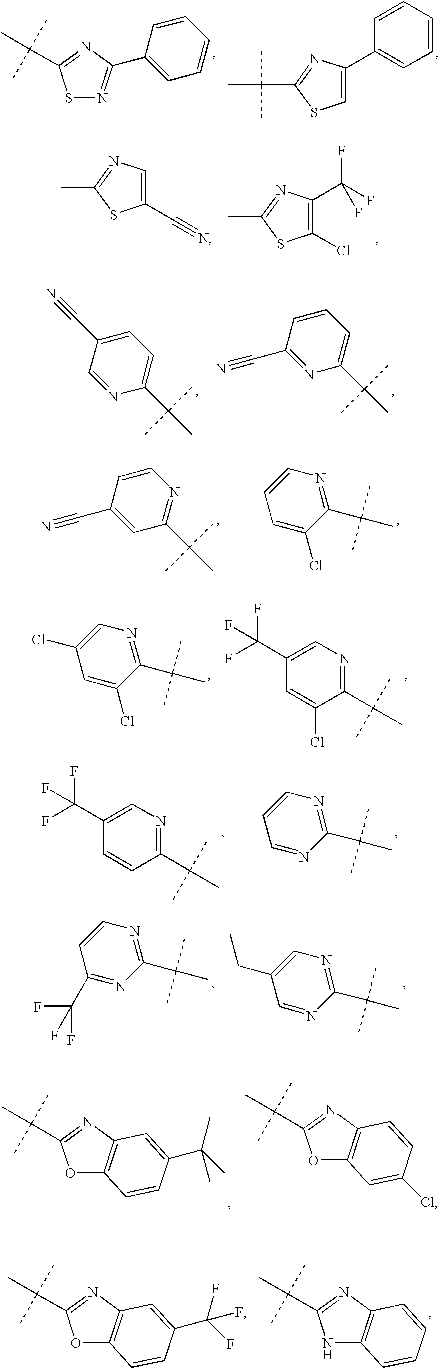

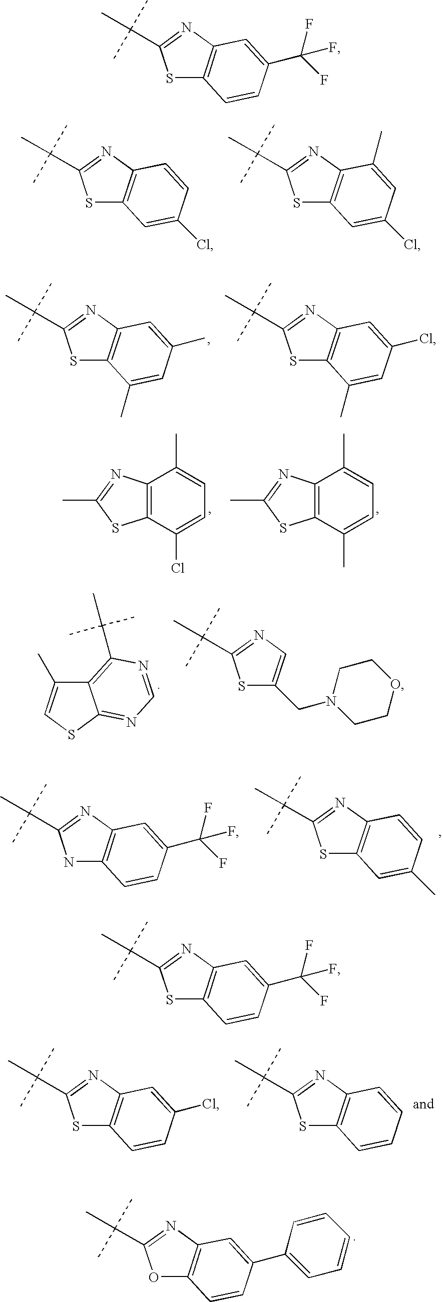

5. A compound chosen from
1-[4-(5-tert-Butyl-benzooxazol-2-yl)-[1,4]diazepan-1-yl]-2-cyclopentyl-ethanone;
[4-(5-tert-Butyl-benzooxazol-2-yl)-[1,4]diazepan-1-yl]-(tetrahydro-pyran-4-yl)-methanone;
[4-(5-tert-Butyl-benzooxazol-2-yl)-[1,4]diazepan-1-yl]-(4-chloro-phenyl)-methanone;
[4-(5-tert-Butyl-benzooxazol-2-yl)-[1,4]diazepan-1-yl]-(4,4-difluoro-cyclohexyl)-methanone;
(Tetrahydro-pyran-4-yl)-[4-(5-trifluoromethyl-benzooxazol-2-yl)-[1,4]diazepan-1-yl]-methanone;
4-(3-Chloro-5-trifluoromethyl-pyridin-2-yl)-[1,4]diazepane-1-carboxylic acid (4-methyl-thiazol-2-yl)-amide;
4-(3-Chloro-5-trifluoromethyl-pyridin-2-yl)-[1,4]diazepane-1-carboxylic acid (tetrahydro-pyran-4-ylmethyl)-amide;
[4-(6-Chloro-benzothiazol-2-yl)-[1,4]diazepan-1-yl]-(tetrahydro-pyran-4-yl)-methanone;
[4-(6-Chloro-benzothiazol-2-yl)-[1,4]diazepan-1-yl]-(4-chloro-phenyl)-methanone;
[4-(3-Phenyl-[1,2,4]thiadiazol-5-yl)-[1,4]diazepan-1-yl]-(tetrahydro-pyran-4-yl)-methanone;
(4-Chloro-phenyl)-[4-(3-phenyl-[1,2,4]thiadiazol-5-yl)-[1,4]diazepan-1-yl]-methanone;
4-(3-Chloro-5-trifluoromethyl-pyridin-2-yl)-[1,4]diazepane-1-carboxylic acid (5-tert-butyl-isoxazol-3-yl)-amide;
4-(3-Chloro-5-trifluoromethyl-pyridin-2-yl)-[1,4]diazepane-1-carboxylic acid (4-tert-butyl-thiazol-2-yl)-amide;
4-(3-Chloro-5-trifluoromethyl-pyridin-2-yl)-[1,4]diazepane-1-carboxylic acid (5-tert-butyl-4-methyl-thiazol-2-yl)-amide;
4-(3-Chloro-5-trifluoromethyl-pyridin-2-yl)-[1,4]diazepane-1-carboxylic acid (2-tert-butylsulfanyl-ethyl)-amide;
4-(3-Chloro-5-trifluoromethyl-pyridin-2-yl)-[1,4]diazepane-1-carboxylic acid (5-tert-butyl-2-methyl-2H-pyrazol-3-yl)-amide;
(4-Trifluoromethyl-phenyl)-[4-(5-trifluoromethyl-pyridin-2-yl)-[1,4]diazepan-1-yl]-methanone;
4-(5-Trifluoromethyl-pyridin-2-yl)-[1,4]diazepane-1-carboxylic acid (4-trifluoromethyl-phenyl)-amide;
6-Chloro-2-[4-(3-chloro-5-trifluoromethyl-pyridin-2-yl)-[1,4]diazepan-1-yl]-benzothiazole;
1-(3-Chloro-5-trifluoromethyl-pyridin-2-yl)-4-(3-phenyl-[1,2,4]thiadiazol-5-yl)-[1,4]diazepane;
4-(3,5-Dichloro-pyridin-2-yl)-[1,4]diazepane-1-carboxylic acid (5-tert-butyl-isoxazol-3-yl)-amide;
4-Pyrimidin-2-yl-[1,4]diazepane-1-carboxylic acid (5-tert-butyl-isoxazol-3-yl)-amide;
4-(4-Trifluoromethyl-pyrimidin-2-yl)-[1,4]diazepane-1-carboxylic acid (5-tert-butyl-isoxazol-3-yl)-amide;
4-(5-Trifluoromethyl-pyridin-2-yl)-[1,4]diazepane-1-carboxylic acid (5-tert-butyl-isoxazol-3-yl)-amide;
4-(3-Chloro-pyridin-2-yl)-[1,4]diazepane-1-carboxylic acid (5-tert-butyl-isoxazol-3-yl)-amide;
4-Pyrimidin-2-yl-[1,4]diazepane-1-carboxylic acid (5-tert-butyl-2-methyl-2H-pyrazol-3-yl)-amide;
4-(5-Trifluoromethyl-pyridin-2-yl)-[1,4]diazepane-1-carboxylic acid (5-tert-butyl-2-methyl-2H-pyrazol-3-yl)-amide;
4-(3,5-Dichloro-pyridin-2-yl)-[1,4]diazepane-1-carboxylic acid (5-tert-butyl-2-methyl-2H-pyrazol-3-yl)-amide;
4-(6-Chloro-9H-purin-2-yl)-[1,4]diazepane-1-carboxylic acid [5-tert-butyl-isoxazol-(3E)-ylidene]-amide;
(1,1-Dioxo-1□⁶-thiomorpholin-4-yl)-[4-(5-trifluoromethyl-benzooxazol-2-yl)-[1,4]diazepan-1-yl]-methanone;
Morpholin-4-yl-[4-(5-trifluoromethyl-benzooxazol-2-yl)-[1,4]diazepan-1-yl]-methanone;
(4-Hydroxy-cyclohexyl)-[4-(5-trifluoromethyl-benzooxazol-2-yl)-[1,4]diazepan-1-yl]-methanone;
(4-Benzothiazol-2-yl-[1,4]diazepan-1-yl)-morpholin-4-yl-methanone;
2-[4-(Tetrahydro-pyran-4-carbonyl)-[1,4]diazepan-1-yl]-thiazole-5-carbonitrile;
[4-(5-Chloro-4-trifluoromethyl-thiazol-2-yl)-[1,4]diazepan-1-yl]-morpholin-4-yl-methanone;
[4-(5-Chloro-4-trifluoromethyl-thiazol-2-yl)-[1,4]diazepan-1-yl]-(tetrahydro-pyran-4-yl)-methanone;
(4-Benzothiazol-2-yl-[1,4]diazepan-1-yl)-(tetrahydro-pyran-4-yl)-methanone;
[4-(5-Chloro-4-methyl-benzothiazol-2-yl)-[1,4]diazepan-1-yl]-morpholin-4-yl-methanone;
[4-(7-Chloro-4-methyl-benzothiazol-2-yl)-[1,4]diazepan-1-yl]-morpholin-4-yl-methanone;
[4-(5,7-Dimethyl-benzothiazol-2-yl)-[1,4]diazepan-1-yl]-morpholin-4-yl-methanone;
[4-(5-Methoxy-benzothiazol-2-yl)-[1,4]diazepan-1-yl]-morpholin-4-yl-methanone;
[4-(6-Chloro-4-methyl-benzothiazol-2-yl)-[1,4]diazepan-1-yl]-(tetrahydro-pyran-4-yl)-methanone;
[4-(7-Chloro-4-methyl-benzothiazol-2-yl)-[1,4]diazepan-1-yl]-(tetrahydro-pyran-4-yl)-methanone;
[4-(5-Methoxy-benzothiazol-2-yl)-[1,4]diazepan-1-yl]-(tetrahydro-pyran-4-yl)-methanone;
[4-(4-Methyl-benzothiazol-2-yl)-[1,4]diazepan-1-yl]-(tetrahydro-pyran-4-yl)-methanone;
[4-(4,7-Dimethyl-benzothiazol-2-yl)-[1,4]diazepan-1-yl]-(tetrahydro-pyran-4-yl)-methanone;
[4-(5,7-Dimethyl-benzothiazol-2-yl)-[1,4]diazepan-1-yl]-(tetrahydro-pyran-4-yl)-methanone;
[4-(5-Chloro-4-methyl-benzothiazol-2-yl)-[1,4]diazepan-1-yl]-(tetrahydro-pyran-4-yl)-methanone;
2-[4-(3-Chloro-5-trifluoromethyl-pyridin-2-yl)-[1,4]diazepan-1-yl]-5-trifluoromethyl-benzooxazole;
[4-(5-tert-Butyl-benzooxazol-2-yl)-[1,4]diazepan-1-yl]-morpholin-4-yl-methanone;
[4-(5-tert-Butyl-benzooxazol-2-yl)-[1,4]diazepan-1-yl]-(1,1-dioxo-1,6-thiomorpholin-4-yl)-methanone;
[4-(4-Methyl-thiazol-2-yl)-[1,4]diazepan-1-yl]-(tetrahydro-pyran-4-yl)-methanone;
[4-(4-Phenyl-thiazol-2-yl)-[1,4]diazepan-1-yl]-(tetrahydro-pyran-4-yl)-methanone;
[4-(5-Morpholin-4-ylmethyl-thiazol-2-yl)-[1,4]diazepan-1-yl]-(tetrahydro-pyran-4-yl)-methanone;
5-tert-Butyl-2-[4-(3-chloro-5-trifluoromethyl-pyridin-2-yl)-[1,4]diazepan-1-yl]-benzooxazole;
[4-(1H-Benzoimidazol-2-yl)-[1,4]diazepan-1-yl]-(1,1-dioxo-1,6-thiomorpholin-4-yl)-methanone;
[4-(1H-Benzoimidazol-2-yl)-[1,4]diazepan-1-yl]-(tetrahydro-pyran-4-yl)-methanone;
Morpholin-4-yl-[4-(6-trifluoromethyl-1H-benzoimidazol-2-yl)-[1,4]diazepan-1-yl]-methanone;
(1,1-Dioxo-1□⁶-thiomorpholin-4-yl)-[4-(6-trifluoromethyl-1H-benzoimidazol-2-yl)-[1,4]diazepan-1-yl]-methanone;
(Tetrahydro-pyran-4-yl)-[4-(6-trifluoromethyl-1H-benzoimidazol-2-yl)-[1,4]diazepan-1-yl]-methanone;
[4-(3-Methyl-pyridin-2-yl)-[1,4]diazepan-1-yl]-(tetrahydro-pyran-4-yl)-methanone;
4-(5-Ethyl-pyrimidin-2-yl)-[1,4]diazepane-1-carboxylic acid (5-tert-butyl-2-methyl-2H-pyrazol-3-yl)-amide;
[4-(6-Fluoro-benzothiazol-2-yl)-[1,4]diazepan-1-yl]-(tetrahydro-pyran-4-yl)-methanone;
[4-(6-Methyl-benzothiazol-2-yl)-[1,4]diazepan-1-yl]-(tetrahydro-pyran-4-yl)-methanone;
(Tetrahydro-pyran-4-yl)-[4-(5-trifluoromethyl-benzothiazol-2-yl)-[1,4]diazepan-1-yl]-methanone;
[4-(5-Chloro-benzothiazol-2-yl)-[1,4]diazepan-1-yl]-(tetrahydro-pyran-4-yl)-methanone;
(4,4-Difluoro-cyclohexyl)-[4-(5-trifluoromethyl-benzooxazol-2-yl)-[1,4]diazepan-1-yl]-methanone;
4-(3-Chloro-5-trifluoromethyl-pyridin-2-yl)-[1,4]diazepane-1-carboxylic acid (5-sec-butyl-2-methyl-2H-pyrazol-3-yl)-amide;
[4-(5-Chloro-benzooxazol-2-yl)-[1,4]diazepan-1-yl]-(tetrahydro-pyran-4-yl)-methanone;
[4-(6-Chloro-benzooxazol-2-yl)-[1,4]diazepan-1-yl]-(tetrahydro-pyran-4-yl)-methanone;
[4-(5-Phenyl-benzooxazol-2-yl)-[1,4]diazepan-1-yl]-(tetrahydro-pyran-4-yl)-methanone;
[4-(5-Chloro-benzooxazol-2-yl)-[1,4]diazepan-1-yl]-morpholin-4-yl-methanone;
[4-(6-Chloro-benzooxazol-2-yl)-[1,4]diazepan-1-yl]-morpholin-4-yl-methanone;
Morpholin-4-yl-[4-(5-phenyl-benzooxazol-2-yl)-[1,4]diazepan-1-yl]-methanone;
4-(5-Cyano-pyridin-2-yl)-[1,4]diazepane-1-carboxylic acid (5-tert-butyl-2-methyl-2H-pyrazol-3-yl)-amide;
4-(5-Cyano-pyridin-2-yl)-[1,4]diazepane-1-carboxylic acid (5-tert-butyl-isoxazol-3-yl)-amide;
4-(4,6-Dimethoxy-[1,3,5]triazin-2-yl)-[1,4]diazepane-1-carboxylic acid (5-tert!-butyl-2-methyl-2H-pyrazol-3-yl)-amide;
4-(6-Cyano-pyridin-2-yl)-[1,4]diazepane-1-carboxylic acid (5-tert!-butyl-isoxazol-3-yl)-amide;
4-(5-Ethyl-pyrimidin-2-yl)-[1,4]diazepane-1-carboxylic acid (5-tert-butyl-isoxazol-3-yl)-amide;
4-(4-Cyano-pyridin-2-yl)-[1,4]diazepane-1-carboxylic acid (5-tert-butyl-isoxazol-3-yl)-amide;
4-(4-Methyl-pyrimidin-2-yl)-[1,4]diazepane-1-carboxylic acid (5-tert-butyl-isoxazol-3-yl)-amide;
(4-Furo[3,2-c]pyridin-4-yl-[1,4]diazepan-1-yl)-morpholin-4-yl-methanone;
[4-(4-Fluoro-phenyl)-[1,4]diazepan-1-yl]-morpholin-4-yl-methanone;
[4-(6-Chloro-benzothiazol-2-yl)-[1,4]diazepan-1-yl]-morpholin-4-yl-methanone;
Morpholin-4-yl-[4-(8-trifluoromethyl-quinolin-4-yl)-[1,4]diazepan-1-yl]-methanone;
[4-(5-Methyl-thieno[2,3-d]pyrimidin-4-yl)-[1,4]diazepan-1-yl]-morpholin-4-yl-methanone;
{4-[5-(3,5-Dimethyl-pyrazol-1-ylmethyl)-thiazol-2-yl]-[1,4]diazepan-1-yl}-(tetrahydro-pyran-4-yl)-methanone;
{4-[5-(3,4-Dihydro-2H-quinolin-1-ylmethyl)-thiazol-2-yl]-[1,4]diazepan-1-yl}-(tetrahydro-pyran-4-yl)-methanone;
[4-(5-Methyl-benzooxazol-2-yl)-[1,4]diazepan-1-yl]-(tetrahydro-pyran-4-yl)-methanone; and
[4-(5-Methyl-oxazolo[4,5-b]pyridin-2-yl)-[1,4]diazepan-1-yl]-(tetrahydro-pyran-4-yl)-methanone
or a pharmaceutically acceptable salt thereof.

6. A pharmaceutical composition comprising a therapeutically effective amount of a compound according to claim 1.

7. A method of treating pain in an animal subject comprising administering to said animal subject a therapeutically effective dose of the compound according to claim 1.

8. A method of treating a disease or condition chosen from a lung disease, a rheumatic disease, an autoimmune disease, a musculoskeletal disease, an allergic disease, an allergic reaction, a vascular disease, a dermatological disease, a renal disease, a hepatic disease, a gastrointestinal disease, neurodegeneration eye disease, diseases of the ear, nose, and throat, neurological disease blood disease, tumors, endocrine diseases, organ and tissue transplantations and graft-versus-host diseases, severe states of shock, acute pain, visceral pain, spasm of the gastrointestinal tract or uterus, colics, neuropathic pain, inflammatory and nociceptive pain, cancer pain, headache, restenosis, atherosclerosis, reperfusion injury, congestive heart failure, myocardial infarction, thermal injury, multiple organ injury secondary to trauma, necrotizing enterocolitis and syndromes associated with hemodialysis, leukopheresis, and granulocyte transfusion, sarcoidosis, gingivitis and pyrexia comprising administering to said animal subject a therapeutically effective dose of the compound according to claim 1.
